(19) United States
(12) Patent Application Publication
(10) Pub. No.: US 2018/0216060 A1
(43) Pub. Date: Aug. 2, 2018

(54) METHODS AND MEANS FOR THE MODIFICATION OF CELL SURFACES

(71) Applicant: ACADEMISCH ZIEKENHUIS LEIDEN (ALSO ACTING UNDER THE NAME OF LEIDEN UNIVERSITY MEDICAL CENTER), Leiden (NL)

(72) Inventors: Fijs Willem Bernhard VAN LEEUWEN, Leiderdorp (NL); Marcus Theodorus Maria ROOD, Utrecht (NL)

(73) Assignee: ACADEMISCH ZIEKENHUIS LEIDEN (ALSO ACTING UNDER TH E NAME OF LEIDEN UNIVERSITY MEDICAL CENTER), Leiden (NL)

(21) Appl. No.: 15/745,537

(22) PCT Filed: Jul. 15, 2016
(86) PCT No.: PCT/EP2016/066972

§ 371 Date: Jan. 17, 2018

(30) Foreign Application Priority Data
Jul. 17, 2015 (EP) 15177291.0

(51) Int. Cl. C12N 5/00; G01N 33/532; G01N 33/569

(52) U.S. Cl. CPC C12N 5/0006; G01N 33/56966; G01N 33/532

(57) ABSTRACT

The invention relates to the utilization of specific cell surface biomarkers (preferably cell specific receptors) to generate a reversible layer-by-layer scaffold for the functionalization of cells. The cells of the present invention can be used in a wide variety of distinct applications, including cellular targeting, imaging, drug delivery, purification, cell sensing, diagnosis and waste stream/body fluid purification.

### FIELD OF THE INVENTION

The invention relates to the fields of cell biology and medicine. In particular the invention relates to the field of biochemical modifications of cell surfaces, to cells with modified cell surfaces and to a wide variety of methods that use the cells of the invention in multiple applications including imaging, drug targeting, cell selection (e.g. for diagnostic purposes, sensing and/or purification methods), tissue engineering and targeted cell delivery.

### BACKGROUND ART

Living cells are the ideal therapeutic tools. Next to being stable and biocompatible, they can perform dozens of different functions simultaneously depending on internal and external factors. And above all, cells are able to self-replicate. Many different types of cells have been encapsulated to control their interaction with the biological environment e.g. decrease interactions with the immune system. Examples include the encapsulation of insulin-producing pancreatic islets, cells that express all kinds of growth factors, and cells that express enzymes for the destruction/removal of diseased tissue. For encapsulated pancreatic islets, transplantation can be performed with a high local accuracy, and these cells will remain at the site of transplantation more effectively. For other applications, such as tissue renewal or the removal of diseased tissue, a local injection is generally much more difficult. In an ideal situation, cells would find their own way to the site where they are needed. Exploring this ‘homing’ concept in during the therapeutic administration of cells is a relatively new and very promising field of research. Promising cell therapies where such homing properties may significantly enhance the therapeutic effectively include the use of stem cells, macrophages and T-cells. The diseased location the cells should migrate towards could in theory be anywhere in the human anatomy, but most research is focused on tissue reconstruction (e.g. infarcted areas) or the treatment of diseased tissue (e.g. cancerous tissue). Currently a lack of affinity of the threapeutic cells for their target tissue limits their retention and thus therapeutic potential. Targeting of these therapeutic cells to specific areas within the patients anatomy may provide outcome. However, such guidance remains complex and mechanical or genetic manipulation of the cell surface is required to guide the homing process (Sohni and Verfaillie. Mesenchymal stem cells migration homing and tracking. Stem cells international 2013 (2013)130763-130763; Kershaw et al. Supernatural t cells: Genetic modification of t cells for cancer therapy. Nature Reviews Immunology 2005 (5):928-940)

In the human body, homing of stem cells and immune cells is mainly accomplished by chemotaxis, an effect that is driven by the interactions between (donor) molecules residing on the cell surface and (receptor) molecules excreted or presented at the cell surface at e.g. damaged tissue in need of regeneration. The Holy Grail in cell therapy would be to use chemotaxis in a controlled manner. It would for example be groundbreaking if controlled migration of stem cells through the body to reach the location of choice, where the stem cells attach and perform their activities (such as tissue re-generation in situ), can be generated. A number of studies acknowledge this concept (Ansboro et al. Strategies for improved targeting of therapeutic cells: Implications for tissue repair. European Cells & Materials 2012 (23):310-319; Kean et al. Development of a peptide-targeted, myocardial ischemia-homing, mesenchymal stem cell. Journal of Drug Targeting 2012 (20):23-32, Li et al. Training stem cells for treatment of malignant brain tumors, World J Stem Cells. 2014 6(4): 432-440.). To achieve this concept, there is a strong need for a controllable and specific method to functionalize cell surfaces, especially the surfaces of therapeutic cells. The disadvantage of the published cell-homing approaches is that they concern surface modifications (of the stem cells) that are either non-specific or non-reversible, both may negatively influence the cells' long-term viability and/or therapeutic impact. Ideally, cell modifications that enhance artificial chemotaxis have limited influence on the cells' viability and therapeutic capabilities. These features may be provided by specific and reversible cell-surface modifications.

There are many different possibilities to modify the surface of living cells. The most ethically complex approach is through genetic modification of the cells. A wide variety of different membrane receptors, proteins, or reactive ligands such as azides can be artificially (over)expressed at the cell's surface through modification of the cell's genetic profile. A more translational means to obtain some sort of surface modification is through molecular training; immune cells can be trained to recognize a specific disease related receptor by exposing them to disease-specific proteins or small molecules. The latter approach is successfully used in immunotherapy, but can be cumbersome and provides limited control over the modifications induced. Some results with training of stem cells are also reported recently (Li et al. Training stem cells for treatment of malignant brain tumors, World J Stem Cells. 2014 6(4): 432-440).

Some alternative (chemical) methods to genetic modification and cell training exist. In general, the different approaches can be divided in three basic categories:

### Not Cell-Specific, Non-Reversible

Examples of this method include the conjugation of functional groups to the proteins residing within the cell membrane using epoxides or activated esters (Sarkar et al. Chemical engineering of mesenchymal stem cells to induce a cell rolling response. Bioconjugate Chemistry 2008 (19):2105-2109; Dong et al. Immuno-isolation of pancreatic islet allografts using pegylated nanotherapy leads to long-term normoglycemia in full mhc mismatch recipient mice. PloS ONE 2012 (7): e50265; Mathapa and Paunov. Fabrication of viable cyborg cells with cyclodextrin functionality. Biomaterials Science 2014 (2):212-219). These types of modifications are irreversible and have no cell specificity; they will bind to any primary amine exposed on any cell membrane. This technique has allowed for the generation of three dimensional culturing of cells. Yet another method uses the cell's metabolism to introduce azide groups on the surface (Gartner and Bertozzi. Programmed assembly of 3-dimensional microtissues with defined cellular connectivity. Proc Natl Acad Sci USA 2009 (106):4606-4610). This was used to place DNA-strands on the outer surface of cells by click chemistry. Cells with complementary DNA strands allow cell-cell interactions and even orthogonal DNA-DNA interactions with which up to three differently modified cells can be connected. All methods described above are not cell-specific and the introduced modification will stay on the cell membrane for the remaining lifetime of the cell.

### Not Cell-Specific, Reversible

In this category, one of the approaches uses the apolar areas created by functionalized lipids, as a non-specific binding site for hydrophobic molecules. Palmitoylation of proteins and peptides provides the proteins and peptides with an aliphatic tail. The palmitoyl group inserts itself at a random position within the hydrophobic shell created by the phopspholipid groups that make up the cell membrane. When the palmitoyl group binds to the lipid membrane, an attached protein/peptide structure will most likely reside on the outside of the cell membrane (Dennis et al. Targeted delivery of progenitor cells for cartilage repair. Journal of Orthopaedic Research 2004 (22):735-741). When a competing hybrophobic binding site is provided e.g. in the form of another cell membrane or a lipid environment, this method allows dissociation of the introduced group, thereby restoring cells to their natural state; non-covalent bonds exist in an equilibrium that can be influenced using external parameters. Although this approach should provide reversible cell-functionalization, it does not allow for cell specificity and provides limited control on the degree of functionalization. Another approach in this category is membrane fusion by introducing covalent ‘click’ groups on the cell membrane surface through membrane fusion, specific binding between cells with complementary click groups was achieved (Dutta et al. Synthetic chemoselective rewiring of cell surfaces: Generation of three-dimensional tissue structures. Journal of the American Chemical Society 2011 (133):8704-8713).

Instead of hydrophobic interactions, charge-based interaction can also be used to non-specifically functionalize the cell membrane. In this field, non-specific (multilayer) deposition of polymers on cell surfaces has been extensively investigated. Much research on the polymer coating of cells has been performed through layer-by-layer coating of bacteria and yeast cells using the coulombic interactions of poly-ionic polymers (Cell Surface Engineering, edited by Fakhrullin, Choi and Lvov, R S C, 2014). Hereby the first layer comprises of a positively charged polyelectrolyte that binds to the negatively charged cell wall. This technique was validated on a range of mammalian cells, including stem cells and breast cancer cells (Veerabadran et al. Nanoencapsulation of stem cells within polyelectrolyte multilayer shells. Macromolecular Bioscience 2007 (7):877-882; Germain et al. Protection of mammalian cell used in biosensors by coating with a polyelectrolyte shell. Biosensors & Bioelectronics 2006 (21):1566-1573). One of the main disadvantages of this technique is the lack of selectivity in the labeling process. On top cells tend to be more susceptible to loss of integrity of the membrane when using polycationic compounds. To circumvent cytotoxicity problems, nonionic interactions have also been used to build layers on the cell surface (Carter et al. Truly nonionic polymer shells for the encapsulation of living cells. Macromolecular Bioscience 2011 (11):1244-1253, Kadowaki et al. Control of cell surface and functions by layer-by-layer nanofilms. Langmuir 2010 (26):5670-5678). However, the layer deposition still remains non-specific. Yet, in another concept, the binding of the RGD motif to intergins was used to improve cell proliferation and to attach cells to a preconstructed supramolecular matrix (Park et al. In situ supramolecular assembly and modular modification of hyaluronic acid hydrogels for 3d cellular engineering. Acs Nano 2012 (6):2960-2968). Although a peptide was used that binds to a specific receptor, no selectivity over cell type was achieved, since the peptide was added after cells were immobilized in the matrix.

### Cell-Specific, Non-Reversible

This third method makes use of specific (protein) biomarkers that are expressed on the cell surface. Since the expression of such biomarkers is unique for each cell type—a fingerprint as you will—this approach allows for selective and controlled modification of a specific cell population. Targeting of receptors is common in molecular imaging and drug delivery and one of the reasons for this is that this form of biomarker specificity can even be used in a competitive environment e.g. a cell mixture. Chemical modification of biomarker on the outside of the cell can be used to irreversibly alter the cell membrane. Sackstein et al. developed a method in which a fucosyltransferase specifically altered the chemical structure of specific sugar-moieties, thereby altering the binding affinity of the cells for E-Selectin (Sackstein et al. Ex vivo glycan engineering of cd44 programs human multipotent mesenchymal stromal cell trafficking to bone. Nature Medicine 2008 (14):181-187). However, control on the degree of alteration is limited. Perhaps the most important disadvantage of this approach is again that the covalent modifications will remain on the cell surface for the remaining lifetime of the cell, thereby possibly altering its viability and is functionality.

### Cell-Specific, Reversible

In this most optimal set-up known in the art, bispecific or bi-functional antibodies (=antibodies with two specific binding sites) were previously bound to cell-specific biomarkers. These can selectively bind to a receptor, while at the same time they introduce a targeting group to bind to other cells (Lee et al. Antibody targeting of stem cells to infarcted myocardium. Stem Cells 2007 (25):712-717; Thakur A, Lum L G. Cancer therapy with bispecific antibodies: Clinical experience. Current opinion in molecular therapeutics. 2010; 12(3):340-349.). This two step (pretargeting) staining process is simillar to the secondary staining technique used in immunohistochemistry. The cell-specificity allows for selective modification, while the reversibility allows for the return of the modificed cell to its natural state. However bispecific antibodies still have major disadvantages, predominantly related to extreme production costs, insecure stability, their ability tor trigger an immune reaction and antibodies have the tendency to generate toxicity issues (Garber, Bispecific antibodies rise again, Nature Reviews Drug Discovery 2014 13:799-801).

### Applications of Cell-Surface Modifications

Next to the introduction of artificial cell-cell interactions, as described in a few of the examples above (Dutta et al. Synthetic chemoselective rewiring of cell surfaces: Generation of three-dimensional tissue structures. Journal of the American Chemical Society 2011 (133):8704-8713; Gartner and Bertozzi. Programmed assembly of 3-dimensional microtissues with defined cellular connectivity. Proc Natl Acad Sci USA 2009 (106):4606-4610), the similar types of chemistry can be used to control the binding of (eukaryotic) cells to other entities such as surfaces (e.g. in microfluidic devices). In this way, cell-surface modification technologies can be used to prepare e.g. sensors. (Yang et al. Switchable host-guest systems on surfaces. Accounts of Chemical Research 2014 (47):1950-1960; Pieters. Intervention with bacterial adhesion by multivalent carbohydrates. Medicinal Research Reviews 2007 (27); 796-816, Yang et al. Supramolecular chemistry at interfaces: Host-guest interactions for fabricating multifunctional biointerfaces. Accounts of Chemical Research 2014 (47):2106-2115; Brinkmann et al. About supramolecular systems for dynamically probing cells. Chemical Society Reviews 2014 (43):4449-4469). Different interactions can be used simultaneously e.g. in a microfluidic device for an application such as cell profiling (Balakrishnan et al. Node-pore sensing enables label-free surface-marker profiling of single cells. Analytical Chemistry 2015 (87):2988-2995). Reversible cell-surface interactions were also achieved (An et al. A supramolecular system for the electrochemically controlled release of cells. Angewandte Chemie-International Edition 2012 (51):12233-12237; Voskuhl et al. Optical control over bioactive ligands at supramolecular surfaces. Chemical Communications 2014 (50):15144-15147; Gong et al. Photoresponsive “smart template” via host-guest interaction for reversible cell adhesion. Macromolecules 2011 (44):7499-7502). One other system is based on the use of polyrotaxanes to yield non-covalent (but non-reversible) interactions to modify a surface for increased cell binding (Seo et al. Inducing rapid cellular response on rgd-binding threaded macromolecular surfaces. Journal of the American Chemical Society 2013 (135):5513-5516). However, all the above described methods use modifications on a surface to induce cell specificity rather than use modification on the cell to induce specificity for a surface. Modification of the cell surface to induce binding has also been proven possible in a few examples. Biotinylation of cell surface was used in a few examples where both the cells and the surface were modified. The biotin can bind to streptavidin and ternary compounds can also be incorporated to control binding of the cell to the surface (Vermesh et al. High-density, multiplexed patterning of cells at single-cell resolution for tissue engineering and other applications. Angewandte Chemie-International Edition 2011, 50, 7378-7380, Sarkar et al. Engineered mesenchymal stem cells with self-assembled vesicles for systemic cell targeting. Biomaterials 2010, 31, 5266-5274). However, since introduction of biotin to the cell membrane was achieved either by reaction of an active ester or by membrane fusion, there was no cell specificity.

The combination of cell-surface interaction and cell-cell interaction can be used for tissue engineering. Alternating layers of fibronectin and gelatin have been used to coat cells. This technique can be applied to build layers of cells (Matsusaki et al. Fabrication of cellular multilayers with nanometer-sized extracellular matrix films. Angewandte Chemie-International Ed 2007 (46):4689-4692) or in a larger scale to individually coat cells to form one large cluster (Nishiguchi et al. Rapid construction of three-dimensional multilayered tissues with endothelial tube networks by the cell-accumulation technique. Advanced Materials 2011 (23):3506).

Next to the introduction of targeting/binding groups, other possibly useful groups can be attached to the surface. One of the options is to attach diagnostic label to the cell surface. With the right imaging modality it is possible to track introduced (targeted) cells within the human anatomy. Tracking will provide insight in the in vivo pathways and provide guidelines for future improvements.

Alternatively, groups that prevent interactions with the immune system can be introduced to the membrane. Therapeutic cells should also be protected from the immune system; an immune response against the therapeutic cells may neutralize their therapeutic benefit. Polymer-based cell encapsulation technologies be used to hide the cells from the environment, while additionally providing some sturdiness against physical manipulations (Gardner et al. Poly(methyl vinyl ether-alt-maleic acid) Polymers for cell encapsulation 2011, 22 (16) 2127-2145).

In short, to achieve homing of cells to a desired location, targeting groups should be attached to the cell's surface. There are many different ways to introduce new groups to the surface of cells, but most of them are either non-cell-specific or non-reversible. There is a clear need for a method to introduce groups to the surface that is controllable, reversible, and cell-specific.

### SUMMARY OF THE INVENTION

The present invention relates to a functionalized cell comprising a cell surface biomarker bound to a ligand, wherein said ligand is linked to a first guest molecule, and wherein said first guest molecule is non-covalently bound to a host functional group that is part of a first multivalent host structure, wherein the first multivalent host structure forms a first layer of functionalization. Said cells may be maintained, or cultured in vitro, but may also be present in vivo. For many purposes (as disclosed herein) the cells of the invention are preferably in vitro cells. The cell surface biomarker is preferably a receptor present (or expressed) on the cell surface of said cell. The biomarker (or the receptor) determines the selection of the cell that is functionalized.

In a further embodiment, the cell comprises one or more further layers of functionalization, wherein subsequent layers of functionalization are formed by alternating layers of multivalent host and guest structures which are non-covalently bound to one another.

In a preferred embodiment, an outer layer of functionalization formed by a multivalent host or guest structure, hereinafter referred to as outer layer of host-guest functionalization, is further functionalized with one or more functional end-groups.

In yet a further embodiment, a host functional group, which is present in said first multivalent host structure is non-covalently bound to a second guest molecule that is linked to a functional end-group. The cells according to the present invention can be used in a wide variety of applications. Hence, the functional end-group may therefore relate to a wide variety of molecules. Preferably, said functional end-group is an imaging label, a targeting group, a therapeutic group, a nanoparticle, an organic surface, an inorganic surface, a surface of another cell, or a cloaking group.

The present invention also relates to a method for the generation of cells according to the invention. The generation of cells according to the invention is referred to as lunctionalizing cells' because the method adds to the functionality of the cells of choice. The cells can perform functions that they could not perform before they were functionalized as disclosed herein. Hence, the present invention is also directed to a method of functionalizing cells, comprising the steps of:

a) Maintaining a selection of cells in vitro, wherein said cells are selected based on the presence of a cell surface biomarker of choice on the cell surface of said cells;
b) Incubating said cells with a ligand that is able to bind to said cell surface biomarker, wherein said ligand is linked to a first guest molecule;
c) Allowing the ligand to interact with said cell surface biomarker;
d) Incubating said cells with a first multivalent host structure; and
e) Allowing the first multivalent host structure to non-covalently interact with said first guest molecule; in order to obtain functionalized in vitro cells comprising a first layer of functionalization.

In a preferred embodiment, the methods of the present invention further comprise the steps of:

j) Incubating the cells resulting from step e) with a second guest molecule that is linked to a functional end-group; and
k) Allowing said second guest molecule to non-covalently interact with said first or second multivalent host structure.

The invention also relates to a method of targeting selected cells to an organic surface, an inorganic surface or a surface of another cell, said method comprising the steps of:

a) Functionalizing the cells according to the methods of the invention; and
b) Targeting said cells to the organic surface, inorganic surface, or surface of another cell, wherein said surfaces comprise a target recognized by the one or more targeting groups introduced on the surface of the cell.

In another aspect, the invention relates to a use of a cell according to the invention in: cell tracking, such as imaging; targeted delivery, such as (artificial) chemotaxis, immune therapy, tissue engineering, sensing and purification.

### BRIEF DESCRIPTION OF THE DRAWINGS

FIG. 1 shows an overview of different cellular functionalization steps according to the present invention. A cell that expresses a specific biomarker on the outer cell surface is incubated with a binding entity (generally the receptor's natural ligand, a mimic or a binding part thereof) functionalized with a specific guest molecule. The binding entity (also called ligand) interacts with the biomarker, thereby presenting the guest molecules at the cell surface and providing a pre-target layer of functionalization. The cell is subsequently incubated with a multivalent host structure that through the multivalent character links the multiple guest molecules (middle row), providing a first layer of functionalization. In this process some host (and guest) functional groups may remain freely available. Subsequently a first guest structure (preferably also multivalent to allow multi-layering and stronger interactions) is introduced to bind to the free hosts-groups artificially expressed on the cell-surface, thereby generating a second layer of functionalization. Layer-by-layer functionalization of cells may directly be used to introduce groups that induce cell-cell or cell-surface interactions. When the layer-by-layer functionalization process is used in a repetitive form, it can help to amplify the number of functionalizations (host or guest molecules, diagnostic labels, specific biomarker binding groups, or cloaking groups that hide the cells form the immune system) in relation to the number of receptors expressed on the cell surface. The bottom row shows that the layered functionalization process can not only occur with single molecules, but can even be scaled to functionalized surfaces. Combined, these functionalizations can be used to increase the cells utility in diagnostic, therapeutic or tissue engineering applications.

FIG. 2 illustrates the principle of targeting cells that have been artificially functionalized according to the methods of the present invention, to a specific type of other cells, a concept that can be used to target cells to a predetermined location in vivo. Similar as outlined in FIG. 1, cells with receptors in the membrane are incubated with a binding partner (such as a ligand or a part thereof) that is linked to a first guest molecule which subsequently binds to a first multivalent host structure (comprising multiple first host functional groups) forming a non-covalent host-encapsulation around the cell. As a second step, a second guest molecule functionalized with a targeting group is introduced. Through this functionalization process, the cell-surface is functionalized with end-groups tailored to induce a specific type of cell-cell interaction, which can be exploited in local or intravenous application. In a subject in need of (therapeutic) cell therapy, the cells will be artificially altered so they are able to bind to specific cells in the diseased area of interest, for instance in the heart, as displayed here.

FIG. 3 shows a schematic illustration of the experiment in which the need for multivalence was illustrated (see example 2). Cells expressing a specific type of receptor (in this proof-of-principle experiment the Chemokine Receptor 4 (CXCR4)) were incubated with the guest-containing CXCR4-specific peptide (Ac-TZ14011-Ad) as the guest-modified ligand. Subsequently, the cells were incubated with either a multivalent cyclodextrin host structure (polymeric scaffold containing multiple cyclodextrin groups and a Cy5 fluorophore) as a first multivalent host structure, or with a monovalent cyclodextrin functionalized with the same fluorophore. Microscopic visualization of the signal emitted by the Cy5 fluorophore revealed a clear overlap between the polymeric structure and the location of the CXCR4 receptor on the membrane of the cell, whereas no overlap could be detected when the monovalent cyclodextrin was used. This illustrates that multivalent interactions are key in the proposed invention.

FIG. 4 illustrates the reversibility of the host-guest interactions. Via a guest-functionalized biomarker-specific entity (Ac-TZ14011-Ad) (also called guest-modified ligand), cells were initially coated with a Cy5 labeled cyclodextrin polymer (see example 3). This binding could be reversed by competition with a different cyclodextrin polymer that was functionalized with a Cy3 fluorophore. Upon a certain concentration of the competitor (±104 nM) the Cy5 signal decreased, while the Cy3 signal simultaneously increased, indicating an exchange of one polymer for the other.

FIG. 5 Schematically illustrates the attachment of (nano)particles to a multivalent guest or host layer present on the cell membrane according to the present invention (see example 4). In the experiment outlined in FIG. 5, cyclodextrin (host)-coated quantums dots bound to the surface of CXCR4 over-expressing cells that were incubated with Ac-TZ14011-Ad (targeting guest molecule (also called guest-modified ligand) in the form of adamantane functionalized Ac-TZ14011) prior to quantum dot labeling. The necessity of membranous receptor expression and binding-selectivity was underlined by the fact that under identical conditions no staining was observed in parental cells with basal levels of CXCR4 expression.

FIG. 6 shows the proof of concept that this method can also be applied in the binding of coated cells to a (glass) surface containing target specific host groups (see example 7). As shown in the examples that illustrate the broad concept, CXCR4-expressing cells were incubated with Ac-TZ14011-Ad and subsequently these cells were exposed to the glass surface. Binding of cells was observed in the area on the glass surface that was modified with host groups. The confocal image of the cells that bound in vitro is not shown but the binding of cells is schematically given on the right.

FIG. 7 shows a schematic illustration of the interaction between functionalized cells with functionalized bacteria shown in example 8. CXCR4-expressing cells were incubated with Ac-TZ14011-Ad, and subsequently with Cy5 labeled cyclodextrin polymer. In a different vial, bacteria (S. Aureus) were incubated with UBI-Ad2. Modified bacteria were added to the modified cells. After washing, the positive experiment (where bacteria were adamantane-modifed and cells were cyclodextrin-modified) showed significantly higher binding of bacteria to cells compared to control experiments.

FIG. 8 shows a schematic illustration of the interaction between two complementarily modified cells shown in example 9. The top row shows the functionalization of CXCR4-expressing cells with Ac-TZ14011-Ad to produce adamantane-functionalized cells. The bottom row shows the functionalization of CXCR4-expressing cells with Ac-TZ14011-Ad, and subsequently with Cy5 labeled cyclodextrin polymer to produce cyclodextrin-functionalized cells. Mixing of the adamantane-functionalized cells with cyclodextrin-functionalized cells leads to controlled intercellular interactions.

FIG. 9 shows the chemical structures of a number of examples of possible guest molecules that can be used for the generation of cells according to the present invention. Examples that are presented are UBI-Ad2 (FIG. 9A), Cy5-Ad2 (FIG. 9B) and Ac-TZ14011-Ad (FIG. 9C). See example 1 for more details.

FIG. 10 shows the chemical structures of possible host molecules that can be used for the generation of cells according to the present invention. FIG. 10A shows a multivalent cyclodextrin polymer with an imaging label (fluorescent dye) as the functional end-group is presented. FIG. 10B shows a monovalent cyclodextrin attached to Cy5 is presented. See example 1 for more details.

FIG. 11: Analytical HPLC of Ac-TZ14011-Ad.

FIG. 12: A) Diffusion-filtered NMR spectra of Cy3_{1.5}CD₇₂PIBMA₃₈₉, Cy5_{0.5}CD₁₀PIBMA₃₉ and Cy5_{0.4}PIBMA₃₉. B) 1H-NMR spectrum of Cy5_{0.5}CD₁₀PIBMA₃₉ showing integration of the β-CD (β-cyclodextrin) peaks at 5.1 ppm (—O—CH—O—CH—) and at 3.92-3.67 ppm (all other β-CD protons), and the polymer peaks at 1.36-1.07 ppm (—(CH3)2-C—CH2-). The later integral was set at 8 to calculate the β-CD per polymer ratio by dividing the integrals at 5.1 and 3.92-3.67 ppm by 7 and 42 respectively (in theory, if one β-CD per subunit would have been present, their integrals would have been 7 and 42).). C) DOSY spectra of Cy5_{0.5}CD₁₀PIBMA₃₉ (indicated by 40, 50, 60 and 70) and Cy3_{1.5}CD₇₂PIBMA₃₈₉ (indicated by 10, 20, 30 and 70). Vertical axis shows diffusion constant, with the largest molecules appearing at the bottom of the graph. The solvent peak at 4.79 ppm was applied as reference for the diffusion constant. D) DOSY spectrum of Cy5_{0.4}PIBMA₃₉.

FIG. 13 Shows confocal microscopy of viable CXCR4 overexpressing MDA-GFP-CXCR4+ cells (with GFP-Tag) functionalized with either Cy5_{0.5}CD₁₀PIBMA₃₉ or Cy3_{1.5}CD₇₂PIBMA₃₈₉. The CXCR4 receptor was first targeted with Ac-TZ14011-Ad, followed by functionalization with fluorescent Cy5_{0.5}CD₁₀PIBMA₃₉ or Cy3_{1.5}CD₇₂PIBMA₃₈₉ via the host-guest interaction between the β-CD molecules and the Ad functionality. With GFP in green, Cy5 (Cy5_{0.5}CD₁₀PIBMA₃₉) in red, and Cy3 (Cy3_{1.5}CD₇₂PIBMA₃₈₉) in blue. Upper row: Cells functionalized with Cy5_{0.5}CD₁₀PIBMA₃₉. From the left: 1) cell surface emitting green light (GFP), 2) cell surface emitting red light (Cy5), 3) cell surface emitting green and red light (GFP+Cy5). Lower row: Cells functionalized with Cy3_{1.5}CD₇₂PIBMA₃₈₉. From the left: 1) cell surface emitting green light (GFP), 2) cell surface emitting blue light (Cy3), 3) cell surface emitting green and blue light (GFP+Cy3).

FIG. 14. Shows the influence of the Ac-TZ14011-Ad peptide on the degree of A) Cy5_{0.5}CD₁₀PIBMA₃₉ or B) Cy5_{0.4}PIBMA₃₉ functionalization. The latter also indicates the role β-CD has in the modification process, since β-CD is missing in the Cy5_{0.4}PIBMA₃₉ polymer. MDA-GFP-CXCR4+ cells were first incubated with either: no peptide, Ac-TZ14011, or Ac-TZ14011-Ad, followed by incubation with either Cy5_{0.5}CD₁₀PIBMA₃₉ (A) or Cy5_{0.4}PIBMA₃₉ (B). The degree of functionalization was quantified by Flow cytometry (dark grey) or Confocal microscopy (light gray). Graphs show the normalized data with the error bars indicating the standard deviations (n=6) and the significance of differences marked with * (p<0.05) or ** (p<0.01).

FIG. 15 shows confocal images showing a comparison of multivalent and monovalent functionalization of viable cell surfaces. MDA-GFP-CXCR4+ cells incubated with Ac-TZ14011-Ad, and subsequently with Cy5_{0.5}CD₁₀PIBMA₃₉ or a mixture of CD and Cy5-CD. With GFP in green and Cy5 in red. Upper row: Cells functionalized with Cy5_{0.5}CD₁₀PIBMA₃₉. From the left: 1) cell surface emitting green light (GFP), 2) cell surface emitting red light (Cy5), 3) cell surface emitting green and red light (GFP+Cy5). Lower row: Cells functionalized with Cy5-CD. From the left: 1) cell surface emitting green light (GFP), 2) no visible emission 3) cell surface emitting green light (GFP).

FIG. 16 shows competition experiments between radiolabeled and non-radiolabeled CDₙPIBMAₘ polymers on MDAMB231 cells, monitored by radioactivity (A-D) and fluorescence (E). A-B) MDA-GFP-CXCR4+ cells functionalized with Cy5_{0.5}CD₁₀PIBMA₃₉, either radiolabelled (A) or non-radiolabelled (B). C-D) MDA-GFP-CXCR4+ cells functionalized with radiolabelled Cy3_{1.5}CD₇₂PIBMA₃₈₉ (C) or non-radiolabeled Cy3_{1.5}CD₇₂PIBMA₃₈₉ (D). As competitor, Cy5_{0.5}CD₁₀PIBMA₃₉ or Cy3_{1.5}CD₇₂PIBMA₃₈₉ were added to the cells functionalized with 99mTc-CDₙPIBMAₘ polymers (A and C), showing partial replacement of the original polymer. In B and D, 99mTc-Cy5_{0.5}CD₁₀PIBMA₃₉ or 99mTc-Cy3_{1.5}CD₇₂PIBMA₃₈₉ were added as competitor to cells functionalized with non-radioactive CDₙPIBMAₘ polymers, showing binding of the competitor. E) Competition of Cy5_{0.5}CD₁₀PIBMA₃₉ with Cy3_{1.5}CD₇₂PIBMA₃₈₉, followed over time by confocal microscopy: Time=0 minutes: Cell surfaces emit red and green light corresponding to GFP and Cy5_{0.5}CD₁₀PIBMA₃₉; Time=6 minutes: Cell surfaces emit red, green and blue light corresponding to GFP, Cy5_{0.5}CD₁₀PIBMA₃₉ and Cy3_{1.5}CD₇₂PIBMA₃₈₉, respectively; Time=12 minutes: As for time=6 minutes the cell surfaces emit red, green and blue light corresponding to GFP, Cy5_{0.5}CD₁₀PIBMA₃₉ and Cy3_{1.5}CD₇₂PIBMA₃₈₉, respectively. However, more of the red light (Cy5_{0.5}CD₁₀PIBMA₃₉) has been replaced by blue light (Cy3_{1.5}CD₇₂PIBMA₃₈₉); Time=18 minutes: Cell surfaces emit green and blue light corresponding to GFP and Cy3_{1.5}CD₇₂PIBMA₃₈₉.

FIG. 17: A) Schematic illustration of introducing a third-generation of surface modification, e.g. Cy5-Ad2. The host-guest interaction of CD-Ad is dynamic and after functionalizing the cell surface with CDₙPIBMAₘ polymers, e.g. Cy3_{1.5}CD₇₂PIBMA₃₈₉ (step 1,2), non-bound β-CD groups should be available to host the second fluorescent label (step 3). B) Confocal images visualizing the introduction of Cy5-Ad2 on Cy3_{1.5}CD₇₂PIBMA₃₈₉ functionalized MDA-GFP-CXCR4+ cells. Images from the left: 1) Cell surfaces emitting green light confirming expression of GFP-labelled CXC4R receptor, 2) Cell surfaces emitting blue light confirming the binding of Cy3_{1.5}CD₇₂PIBMA₃₈₉ on their surfaces, 3) Cell surfaces emitting red light confirming the binding of Cy5-Ad2 to their surfaces.

FIG. 18: A) Schematic overview of inducing cell-cell interactions (3) between β-CD polymer (Cy31.5CD10PIBMA389) functionalized cells (1) and Ad (Ac-TZ14011-Ad) functionalized cells (2) with Hoechst staining (white) B) Representative confocal images of inducing supramolecular cell-cell interactions between variable functionalized MDA-GFP-CXCR4+ cells. With GFP in green, Cy3 in blue and Hoechst in white. C) Average values of the fraction of cell-cell interactions in each test condition. Significance of differences is marked with * (p<0.05) or ** (p<0.01).

FIG. 19 shows a competition assay curve of Ac-TZ14011-MSAP with Ac-TZ14011-Ad, acquired by flow cytometry, to determine binding constant (KD).

FIG. 20 Shows viability of MDA-GFP-CXCR4+ cells measured 24 h after functionalization with either Cy5_{0.5}CD₁₀PIBMA₃₉ (upper graph) or Cy3₁₅CD₇₂PIBMA₃₈₉ (lower graph) at variable polymer concentrations (0-16 μM).

### DETAILED DESCRIPTION OF EMBODIMENTS

As outlined above, the prior-art is limited on the coating of (eukaryotic) cells for biomedical applications. Most of the above described prior-art either use irreversible or non-specific means for functionalization, while specificity for the cells that need to be functionalized (or targeted) and reversibility of the functionalization as a means to minimize the influence on the cells properties are key features.

There appears to be no common technology that can be applied to different types of systems: cell-cell or cell-tissue interactions, cell-bacteria (or virus) interactions and interactions of different kinds of cells to non-biological surfaces. Importantly as well, there appears to be no common system for a wide variety of applications wherein the cell surface itself is engineered, or ‘functionalized’, in a specific, controllable and predictable manner. There is a clear need for a generic and synthetic technology that takes place at the cell surface, that can be tightly controlled (in the sense of cell choice (specificity), the strength of the interaction and multitude of the layers), which is non-toxic and which is reversible such that the cells itself remain unharmed and stay alive, allowing exploitation of their specific function. Especially the latter would be very beneficial in cell therapies and cell targeting settings.

The present invention provides a system wherein the cell surface itself is engineered and wherein non-covalent multivalent bonds are being applied, such that the entire functionalization of the cells would be reversible. The inventors of the present invention have generated a single system, driven by supramolecular interactions that can be applied to most, if not all, kinds of viable cells. The technology is generally non-toxic and its specificity for the cell-type of choice is ‘biomarker-based’. To the best of the inventors' knowledge, there is only one example that uses non-covalent interactions (DNA-DNA) to bind a polymer to receptor-bound complementary groups (Chu et al. Cell surface self-assembly of hybrid nanoconjugates via oligonucleotide hybridization induces apoptosis. Acs Nano 2014 (8):719-730), which describes receptor clustering of CD20 receptors by means of non-covalent DNA-DNA interactions on a polymer backbone. Novel in the present invention is that starting from a receptor-specific first step, one is now able to use the cells for specific purposes, rather than induce their death. This can be a very interesting opportunity in cell-based therapeutics or diagnostics and even during cell isolation out of aqueous solutions.

The present invention also provides a generated a system that allows one to carefully control the number of layers that are applied, thereby enabling the increase or decrease of the interaction capability of the cell of choice. By using the system of the present invention one is now able to use biomarkers (preferably receptors) on a cell surface to increase the functionality of the cell multiple times through layer-by-layer building of more and more interacting molecules, despite a low number of receptors being present on the surface.

The present invention relates to a functionalized cell comprising a cell surface biomarker bound to a ligand, wherein said ligand is linked to a first guest molecule, and wherein said first guest molecule is non-covalently bound to a host functional group that is part of a first multivalent host structure, wherein the first multivalent host structure forms a first layer of functionalization. Said cells may be maintained, or cultured in vitro, but may also be present in vivo. For many purposes (as disclosed herein) the cells of the invention are preferably in vitro cells. The present invention also relates to methods and means for the chemical and functional modification of cell surfaces in order to prepare functionalized cells according to the invention. Disclosed is a technology that transforms the surface of cells into a versatile platform for supramolecular host-guest chemistry. The present invention also relates to the many different possibilities of using functionalized cells according to the invention.

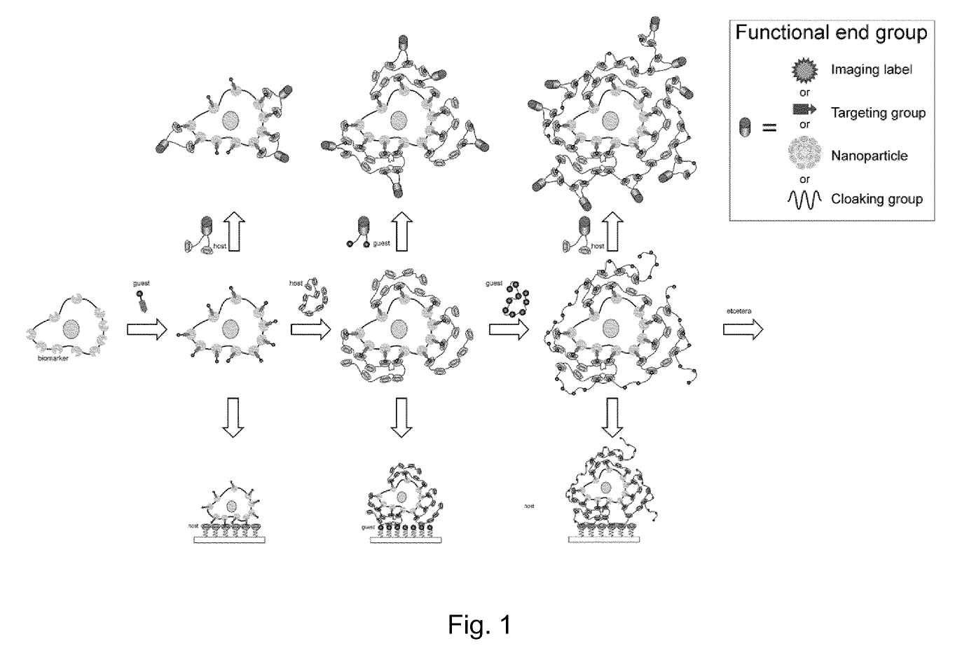

FIG. 1 shows the basic technology of the present invention: a cell of choice, that may be any kind of living cell, is selected for the presence of particular type of biomarker present at the cell surface. A guest molecule functionalized ligand (also called guest-modified ligand), which may be any kind of binding molecule that interacts with the receptor, but which is preferably a synthetically produced peptide that binds to the receptor, and which is linked to a first guest molecule comprising one or more guest functional groups, is applied to bind to the receptor and forms what is here called the pre-target layer of functionalization. The ligand may be recombinantly produced, but is preferably synthetically produced when it is a short peptide. The in vitro synthesis of peptides of no more than about 30 amino acids in length is a technique often used in the art. Covalently attached, often using conventional amino acid coupling techniques, to the peptide in the methods of the present invention is a so-called first ‘guest’ entity (or first guest molecule), which points outwards such that it is available for further binding. In a subsequent step (middle row in FIG. 1) a so-called first ‘host’ structure is introduced that binds to the guest. The guest and host structure interact with one another by virtue of their respective guest and host functional groups, which bind non-covalently and reversible with one another.

The interaction between the first guest molecule and the first host-layer (also called first layer of functionalization) is non-covalent, multivalent and reversible. As further outlined in the accompanying examples and figures, the inventors of the present invention found that at least when using the host-guest pair of adamantane and β-cyclodextrin, the invention does not work appropriately when a monovalent host structure is used; Without wishing to be bound to any theory, this might indicate that the linkage of the receptors through the ligands and the multivalent character of the first host structure is important. However, the need for a the first host structure to comprise more than one host functional group may also be due to that the interaction strength seems to go up with host structures that contain a higher number of host functional groups. Accordingly, in a preferred embodiment of the present invention, the first host structure comprises multiple host functional groups (at least two), wherein each of these groups may or may not interact with a separate first guest molecule linked to the ligand attached to the biomarker of choice in the cell membrane. This multivalent scaffold molecule (the first multivalent host structure) then forms relatively strong, but non-covalent, bonds with the first guest molecules that have been introduced on the cell surface, thereby essentially coating the cell surface. Through this, it is very likely that some receptors become linked via the multivalent host structures. In a third step (see middle row in FIG. 1), another guest-layer (also called second functionalization layer) may be introduced, which can consist of a variety of guest-modified entities, including, molecules, particles, cells and surfaces (also called guest structures herein). Providing availability of enough free (host or guest) functional groups to attach the next layer, this supramolecular layer-by-layer functionalization of the cell surface can be continued beyond two layers, as long as the same or other suitable subsequent host- and guests-molecules are used. Finally, the functionalized cells will contain an outer layer of either host or guest groups, depending on the goal or application.

### Functionalized

A “functionalized cell” as used herein, is a cell whose surface has been altered or engineered and thereby have achieved another function that prior to functionalization. According to the present invention, this is achieved by the introduction of functional groups to the cell surface. Some of the functional groups that are introduced to the surface of the cell according to the present invention are guest and/or host functional groups, providing the cell with the altered function of being capable of binding complementary guest or host functional groups. Once the cell is functionalized with the host-guest chemistry, it can be bound/targeted to other cells or surfaces, which have been functionalized with the complementary host or guest functional groups. This is a new function, which the cell did not have prior to being functionalized. Furthermore, via the host-guest layers of functionalization achieved by the present invention, the cell can be further functionalized with one or more functional end-group, which can be selected such that the functionalized cell can target e.g. other cells or surfaces of choice depending on the functionality of the chosen functional end-group. The functionalization provided by the present invention has the advantage that it is reversible and that the cells are viable while being functionalized. Furthermore, the functionalization provided by the present invention makes it possible to amplify the number of functional groups exposed on the surface. Yet another advantage is that the cell surface can be rendered more homogeneous and thereby unwanted interaction with its surroundings can be controlled.

The term “functionalized” as used herein, refers to an alteration of the cell surface through the chemical (but reversible) modifications as described herein. A cell surface (or a cell) is for example functionalized when the one or more layers of functionalization comprising guest and host layers, respectively, have been built using the cell surface biomarker(s) of choice that has interacted with the ligand and the guest molecule attached thereto. The cell can—after alterations at its surface following the methods of the present invention—perform a different, new, and/or better function than prior to the modification as provided by the present invention. The first functional groups that are introduced to the surface of the cell according to the present invention are guest and/or host functional groups, which provide the cell with the altered function of being capable of binding complementary guest or host functional groups. Once the cell is functionalized with the host-guest chemistry, it can be bound/targeted to other cells or surfaces, which have been functionalized with the complementary host or guest functional groups. Alternatively, the cell can be further functionalized with one or more functional end-group, which can be selected such that the functionalized cell can target e.g. other cells or surfaces of choice depending on the functionality of the chosen functional end-group. A cell can be functionalized for a wide variety of purposes, including cell targeting, sensing, imaging, etc.

### Cells

Many different types of cells may be used in the methods of the present invention, including prokaryotic and eukaryotic cells. The technology makes use of specific cell surface biomarkers that are expressed and/or present at the cell surface. Such cell surface biomarkers dictate the selection of the cell that is being ‘functionalized’ according to the methods of the present invention and that may be used in a wide variety of applications. As used herein, a ‘cell’ is preferably a living cell, and may be any kind of eukaryotic or prokaryotic cell, mammalian, bacterial, yeast, etc. as long as it allows the attachment of a binding compound linked to a first guest molecule. The cell is preferably isolated in single cell suspensions and has a biomarker of interest on/in/at the outer cell surface, wherein the biomarker is attached to or embedded in the cellular membrane. The methods of the present invention wherein binding compounds such as ligands are attached to the biomarker of choice, can be performed in vitro as well as in vivo. For many therapeutics and non-therapeutic applications, it is preferred that the cell of the present invention is a purified in vitro cell or an in vitro cell that was cultured in vitro, for instance from a cell line or from stem cells, or from cells obtained from a (mammalian subject) and present in a sample. For instance, cells might be obtained from a (human) subject, cultured in vitro and treated according to the methods of the present invention, and re-introduced into the same (human) subject, for instance in the treatment of wounds or other types of disorders where re-introduction and cellular targeting is beneficial. The cell of the present invention is preferably part of a population of cells and preferably expresses multiple copies of the biomarker of interest on its outer cell surface. This means that the singular form as used herein also refers to the plural form, hence a cell may be in a population of cells that all have the same characteristics. Of course, such cells may be combined with other kinds of cells rendering a cell mixture of different types of cells. In another embodiment, cells may also be functionalized in vivo. Any type of cell can be used for a wide range of applications, as outlined in more detail below.

### Cell Surface Biomarkers

The cell surface biomarker as used in the present invention may be any kind of molecule present on the cell surface or being embedded in the cell membrane, but is preferably a receptor that is preferably specific for the cell of interest. The biomarker-driven specificity means that any type of cell can be selected, but that the first step in the functionalization process depends on the cellular biomarker that is present at the surface of the cell of choice. Specific biomarkers or combinations of different (specific) cell surface biomarkers, i.e. different types of cell surface biomarkers, may be used to drive the functionalization, thereby enabling a user of the invention to select specific cells and to keep other cells untouched (non-engineered).

The invention depends on the specific interaction of a (synthetic) ligand binding to a cell surface biomarker (often, and preferably a receptor) present on/in/at the cellular membrane, or cell surface. Any receptor binding to a ligand may be applied in the methods of the present invention. However, the interaction between the receptor and the ligand is preferably driven by normal biological function and the choice is preferably on a combination of a receptor with its natural ligand, a derivative or a binding part thereof e.g. a specific amino acid sequence. Such binding part may be a synthetic peptide that still has strong binding capacity to the cell surface biomarker. In other words, preferably a natural ligand or binding part thereof is selected that interacts with its counterpart; the receptor on/in the membrane of a cell of choice and induces no unwanted immune reactions. The selection of the ligand (and its receptor) is non-generic, which means that this step in the functionalization process dictates the specificity (selection of the cell), the application and the purpose of the method.

The term “cell surface biomarker” as used herein thus refers to a molecule (preferably a receptor) that is present in/on/at the cell membrane of a living cell and that is attached to the cell, preferably in its natural form, and that points outwards to be able to interact with a binding partner, such as a ligand as disclosed herein. The cell surface biomarker is preferably selected for the purpose of cell specificity, which means that different cell surface biomarkers may be selected for different purposes and applications. The cell surface biomarker may be any kind of molecule that is present in a cell of choice (wherein the choice of the cell largely depends on the type of cell surface biomarker) and that is able to interact with a ligand or a (synthetic) binding part thereof through which functionalization of the cell can be achieved.

To the best of the knowledge of the inventors, modifications of cell biomarkers and with that cell surfaces of individual viable cells with synthetic targeting moieties functionalized with labels that allow for multimeric non-covalent functionalization have never been shown. Moreover, it appears that thus far also no receptor-specific interactions have been used for the proposed purposes. The inventors decided to initially work with the CXCR4 receptor—the endogenous receptor that drives chemotaxis with CXCL12, as an example and as a proof of concept. However, this concept can be broadened and used with other (disease-related) biomarkers present on all kinds of different cells.

In one embodiment, the functionalized in vitro cell according to the present invention comprises a plurality of cell surface biomarkers each bound to a respective ligand, wherein said ligand is linked to a first guest molecule, and wherein said first guest molecule is non-covalently bound to a host functional group of a multivalent host structure, said multivalent host structure forming a first layer of functionalization. The plurality of cell surface biomarkers may comprise one or more types of cell surface biomarkers (such as different molecules binding different ligands). In another embodiment, the plurality of cell surface biomarkers bound by a guest-modified ligands which in turn are bound the first multivalent host structure, comprise essentially all cell surface biomarkers of a given type.

### Ligand

The term “ligand” as used herein refers to a binding moiety that interacts with a cell surface biomarker (often a receptor) present on the outer surface of the cell membrane. In a preferred embodiment such ligand mimics the natural ligand of the receptor of choice, but the ligand may also be a synthetic compound (representing the binding part of the natural ligand) of any suitable length, as long as it interacts specifically with its receptor. The ligand may also be the natural ligand itself or a binding part thereof. In a preferred embodiment, the ligand is a peptide that specifically interacts with its natural counterpart (=the receptor). In a more preferred aspect said peptide is short enough to be manufactured in vitro (or synthetically) using methods known to the person skilled in the art. Because of the disadvantages indicated above (immune responses, costs, etc.), when the functionalized cell is intended for use in therapy, the ligand is preferably not an antibody, or a (recombinant) part thereof.

In one embodiment, the ligand is selected from the group consisting of a natural ligand of the cell surface biomarker or a binding part thereof, a synthetic peptide capable of binding the cell surface biomarker or a binding part thereof and an inhibitor for the receptor or a binding part thereof.

### Guests and Hosts

The terms “guest” and “host” as used herein refer to two different, but complementary, binding partners that interact with each other (examples are beta-cyclodextrin-adamantane, beta-cyclodextrin-ferrocene, gamma-cyclodextrin-pyrene, cucurbituril-viologen, Ni(NTA)-His tag). Preferably, one guest interacts with one host. In any event, according to the present invention, the interaction between the guest and the host is reversible and non-covalent. In principle a guest molecule does not normally interact with another guest molecule. However, guest functional groups may be interconnected through a scaffold molecule to form a multivalent guest structure.

As used herein, the term “guest functional group” means the part or moiety of a monomer of the guest molecule, which enables the binding to a complementary host functional group. A “guest molecule” is in turn a molecule that comprises one or more guest functional groups, where a monovalent guest molecule comprises one guest functional group and a multivalent guest molecule comprises at least two guest functional groups. As used herein, the term “host functional group” means the part or moiety of a monomer of the host molecule, which enables the binding to a complementary guest functional group. A “host molecule” is in turn a molecule that comprises one or more host functional groups, where a monovalent host molecule comprises one host functional group and a multivalent host molecule comprises at least two host functional groups. As used herein, the term “guest-host molecule interactions” means the non-covalent binding between respective guest and host functional groups. In a preferred setting, hydrophobic interactions, such as lipophilic interactions, are being used instead of interactions that are based on charge.

For the sake of clarity, the present disclosure describes the first guest molecule as the molecule that is linked to the ligand, which interacts with the receptor on the cell membrane, whereas the host molecule or multivalent host structure is then able to bind that guest molecule. The terms could have been reversed (using the host molecule as the molecule that is linked to the ligand), but as used herein, the host molecule or host structure is not linked to the ligand directly, but only via the guest molecule. In a multivalent host structure, comprising multiple host functional groups, some of the host functional groups may be non-covalently bound to a guest molecule, while others remain free. Such free host functional groups within the multivalent host structure may then be available to interact with another guest molecule or functional group thereof, either one that is also bound to the biomarker via a ligand, or one that is present in a new layer, for instance a guest functional group that is present in a multivalent guest structure, which in turn may contain free guest functional groups that enables the skilled person to generate yet another layer with host functional groups, preferably present in a multivalent host structure. The term ‘guest’ and ‘host’ have been chosen to indicate that these molecules or functional groups thereof are the two different molecules that bound to each other (non-covalently) to generate layers. Other terms may also have been chosen, such as ‘master’/‘slave’, or ‘plus’/‘minus’, or ‘plug’/‘socket’, or ‘yin’/‘yang’, etc.

When cyclodextrins are applied in the methods of the present invention, preferably beta-cyclodextrin is used, which is the best binding partner for adamantane. The cyclodextrin may contain additional groups, such as an amine to attach it to a scaffold, one or more thiols to bind the cyclodextrin to a gold surface, or hydroxypropyl groups to increase solubility and biocompatibility. Other members of the cyclodextrin family (most likely alpha and gamma) can also be used for host-guest interaction, although different guests have to be introduced to achieve this. Accordingly, a good, but not the only, example of supramolecular host-guest interactions that can be applied in the invention is the non-covalent interaction between adamantane (as the guest molecule) and β-cyclodextrin (as the host molecule). Adamantane is a small molecule that may be attached to a ligand that recognizes and binds a cellular biomarker. Cyclodextrin-based polymers have been used for therapeutic applications (Kandoth et al. Two-photon fluorescence imaging and bimodal phototherapy of epidermal cancer cells with biocompatible self-assembled polymer nanoparticles. Biomacromolecules 2014 (15):1768-1776) and imaging agents (Yan et al. Poly beta-cyclodextrin inclusion-induced formation of two-photon fluorescent nanomicelles for biomedical imaging. Chemical Communications 2014 (50):8398-8401) and they showed excellent biocompatibility. Combinations of multivalent cyclodextrin- and adamantane-compounds have been used to build a hollow sphere around a vesicle (Samanta et al. Fabrication of hydrophilic polymer nanocontainers by use of supramolecular templates. Journal of the American Chemical Society 2015 (137):1967-1971). One example of coating cells with non-covalently bound polymers includes cells in a matrix built of polymers with pending groups for non-covalent interactions (Park et al. In situ supramolecular assembly and modular modification of hyaluronic acid hydrogels for 3d cellular engineering. Acs Nano 2012 (6):2960-2968), but this is quite different from building layers with non-covalent polymers around the cell surface. It has also been shown that it is possible to covalently (irreversible) attach cyclodextrin groups to the outside of a living yeast cell as a step towards using cell as carriers for drugs and other molecules (Mathapa and Paunov. Fabrication of viable cyborg cells with cyclodextrin functionality. Biomaterials Science 2014 (2):212-219).

Preferably, the guest-host combinations are cheap, enabling one to perform the methods of the present invention on a relatively large scale and/or in low cost devices. In one aspect, the guest molecule may be adamantane, whereas the host molecule is preferably then a cyclodextrin that non-covalently interacts with adamantane. Both compounds are relatively cheap and easy to produce in controlled settings and non-toxic (data not shown). In another preferred embodiment, the guest and/or the host molecules can be attached to a variety of surfaces, such as silica, glass, gold, plastics, or biomaterials. Flexibility in surface modification means that a variety of sensor or purification devices can be used in combination with the functionalization of the cells as disclosed herein. One example is the use of glass/polymer beads for purification/sensing in a solution, or lab-on-chip devices for large array techniques.

### Guest-Modified Ligand as Pre-Targeting Vector

According to the present invention, the ligand is linked to a first guest molecule. Normally, the ligand molecule is covalently linked to a guest molecule, in which case the guest-modified ligand molecule comprises one or more guest functional groups. As used herein, such a ligand which has been linked to a first guest molecule and thereby comprises one or more guest functional groups, is also called the “guest-modified ligand” or a “pre-target vector”. The linking of the guest molecule to the ligand is done by conventional conjugation techniques known to the person skilled in the art. The first guest molecule, which is part of the guest-modified ligand, binds non-covalently to a host functional group in the first multivalent host structure. To be more specific, this interaction takes place by a one to one interaction between a guest functional group in the first guest molecule and a host functional group in the first multivalent host structure. Upon binding of the guest-modified ligand to one or more biomarkers, such as a plurality of biomarkers, a pre-target layer of functionalization is formed on the cell surface. The one or more guest functional groups present in the guest-modified ligand serve as a template on which a subsequent layer of host molecules or host structures can be bound. This plurality of cell surface biomarkers may be of the same type or of different types. When different types of cell surface biomarkers are selected for pre-targeting, they are bound by a respective different type of ligand. In other words, if two different types of cell surface biomarkers are selected, two different types of ligands are most likely needed. The possibility of targeting different types of cell surface biomarkers adds to the specificity by which it is possible to target one specific cell type in a mixture of different cell types. In a further embodiment of the present invention, the plurality of cell surface biomarkers comprise a majority of the one or more types of cell surface biomarkers of choice.

In one embodiment of the present invention, the plurality of cell surface biomarkers comprise essentially all of the one or more types of cell surface biomarkers of choice. Examples are: integrins, PSMA, Endoglin, CD44, g-actin, β-tublin, caveolin, stem cell antigen-1 (SCA-1) and islet-1.

In the examples presented herein, the short peptide Ac-TZ14011, which is known for its specific binding to the C—X—C chemokine receptor type 4 (CXCR4), is used as the ligand for selecting cells, which comprise the CXCR4 receptor. Ac-TZ14011 was linked to adamantane as the first guest molecule resulting in the guest-modifed ligand called Ac-TZ14011-Ad. Accordingly, it has been shown that a monovalent (with respect to guest functional groups) guest-modified ligand is enough to bind the subsequent layer of functionalization comprising host functional groups. The guest-modified ligand according to the present invention comprises at least one guest functional group, but it may comprise more than one.

In a preferred aspect of the invention, the biomarker-specific ligand (which may be any type of entity) is linked to the first guest molecule (adamantane, ferrocence, pyrene, phenyl analogues or any other suitable guest molecule) that is attached to the biomarker on the cell via the ligand, which binds the cell surface biomarker. The adamantane functionalized cells can then be applied as a scaffold to interact with β-cyclodextrin that acts as the host molecule. The cyclodextrin structure is made multivalent, which is important for a proper functionalization. As exemplified in the accompanying examples, adamantane may for instance be linked to Ac-TZ14011 (to form Ac-TZ14011-Ad, as it is named below), which is a short peptide and that binds to CXCR4. The interaction between CXCR4 and Ac-TZ14011 was previously shown to be of use in immunohistochemistry and imaging of tumor cells (Van den Berg N S et al. Immunohistochemical detection of the CXCR4 expression in tumor tissue using the fluorescent peptide antagonist Ac-TZ14011-FITC. Translational Oncology 2011 (4):234-240).

### Multivalent Guest and Host Structures

The term “multivalent” as used herein refers to a number of host or guest molecules, or functional groups thereof, that are part of the same molecule or structure. Multivalent interactions contain at least two functional groups of the same type (e.g. at least two guest functional groups, or at least two host functional groups) bound to each other through a backbone (or scaffold) that allows the multimerization of the matching guest or host molecule. The upper limit in multivalency depends on the purpose of the layer-by-layer manufacturing of guest-host molecule interactions, as disclosed herein.

According to the present invention, a “multivalent host structure” or a “multivalent guest structure”, is a structure comprising at least two host functional groups or guest functional groups, respectively. It can in principle be a dimer or polymer of suitable host or guest monomers, but typically the host or guest molecules have been attached or engrafted onto a polymer of a different type that allows for the attachment of the host or guest molecules. In one embodiment of the present invention, a “multivalent host structure” or “multivalent guest structure” comprises a scaffold structure onto which the at least two host molecules or at least two guest molecules have been attached or engrafted resulting in that the scaffold structure comprises at least two host functional groups or at least two guest functional groups. The scaffold structure can be anything that allows attachment of the host or guest molecules of choice. For example, the scaffold structure can be selected from polymers and nanoparticles, where suitable polymers for example can be selected from poly(isobutylene-alt-maleic anhydride) (PIBMA), PAMAM, poly-acrylic acid, polysaccharides, polypeptides and oligopeptides. Some such polymers, such a PIBMA, has the further advantage that it prevents interaction with the immune system and thereby also functions as a cloaking group. For many applications, it is also of importance that the selected scaffold structure is non-toxic. This can for example be when viability of the functionalized cells is of importance, or where the functionalized cells are intended for administration to a living being. A preferred scaffold molecule that is used to generate multivalent structures containing either the host functional group of cyclodextrin or the guest functional group of adamantane, is poly-isobutylene-alt-maleic anhydride (PIBMA), which can be easily functionalized, is non-toxic, and presents a multitude of negative charges after functionalization which increases water-solubility and provide cloaking. Other preferred scaffold structures that could be applied in the methods of the present invention to generate multivalent structures comprising guest or host functional groups are other polymers containing anhydrides, PAMAM, nanoparticles (e.g. quantum dots), poly-acrylic acid, polysaccharides, organic or inorganic surfaces, cells etc. In another embodiment, the scaffold molecule is a polypeptide comprising less than about 30, such as, e.g. less than about 25, less than about 20, less than about 15, less than about 10, less than about 5 or less than about 6 amino acids. It may also be an oligo peptide such as, e.g. a dipeptide, a tripeptide, a tetrapeptide or a pentapeptide. In one embodiment the repeat unit of the poly or oligepeptide is β-alanine.

### First Multivalent Host Structure

The first host structure, is the host structure, which is non-covalently bound to the first guest molecule via a one to one interaction between a guest functional group of the first guest molecule and a host functional group of the first host structure. The present inventors have shown, that a β-cyclodextrin monomer does not bind to the cells comprising the pre-target layer of functionalization (i.e. the guest-modified ligand). Without wishing to be bound to any theory, it is possible that the need for the first host structure being multivalent is because it bridges at least two receptors by binding to at least two respective guest-modified ligands, which are again bound to at least two respective cell surface biomarkers. However, it is also possible, that the need for the first host structure being multivalent has to do with its affinity for the guest functional groups of the guest-modified ligand. In any event, the first host structure according to the present invention is multivalent, i.e. comprises at least two host functional groups. In a preferred embodiment, the first multivalent host structure comprises at least 3, at least 4, at least 5, at least 6, at least 7, at least 9 or at least 10 host functional groups. In a preferred embodiment, the scaffold structure of the first multivalent host structure is a polymer comprising as least about 5, such as at least about 10, at least about 15, at least about 20, at least about 25, at least about 30, at least about 35 or at least about 40 monomer repeat units. In another embodiment the scaffold polymer is poly(isobutylene-alt-maleic anhydride) (PIBMA). In a further embodiment, the scaffold structure or molecule further comprises one or more functional end-groups, such as, e.g., imaging labels, targeting groups, therapeutic groups, nanoparticles, organic surfaces, inorganic surfaces, the surface of another cell, or cloaking groups. In another embodiment, the first multivalent host structure is connected to at least two different cell surface biomarkers via its non-covalent binding to at least two first guest molecules, wherein the at least two linked guest molecules are linked to a respective ligand which is bound to a respective receptor.

### Further Layers of Functionalization

The subsequent steps in the methods of the invention, wherein the ‘supramolecular cell-coating’ is built on top of the biomarker-targeting ligand is a generic step based on traditional multivalent host-guest chemistry approaches e.g. adamantane—beta-cyclodextrin, which can be applied in aqueous conditions. This functionalization step can be performed independently of the ligand-receptor interaction, and may be used in a wide variety of applications. The host-guest functionalization process may be used several times on the same cell, as outlined below in more detail and shown in the accompanying figures. The same functionalization steps may also be applied on different types of cells, thereby driving uniform interactions with e.g. a sensor irrespective of the biomarker originally expressed at the cell.

Once the functionalized cells according to the invention has been functionalized with a first layer of functionalization (i.e. with the pre-target layer of the guest-modified ligand and next the first multivalent host structure), the cells can be further functionalized either by introducing one or more functional end-groups or by building one or more further layers of host-guest functionalization. The choice depends on the purpose of functionalizing the cells: Building further layers for host-guest functionalization can for example further amplify the number of functional groups, such as host functional groups, guest functional groups or functional end-groups on the surface of the cells. This may be desirable for some purposes, e.g. where the targeted cell surface biomarker is of low abundance on the cell surface. Or vice versa, if the functionalized cell ultimately should bind to a cell or a surface with a low abundance of the target of the introduced targeting end-group. A functionalized cell comprising a first layer of functionalization according to the present invention, comprises a layer of multivalent host structures. Such a cell thereby exposes a plurality of host functional groups on its surface, which, depending on the final desired application of the functionalized cell, can be used for introducing further functionalization of the cell. The host functional groups in first layer of functionalization can be used as a template for building further layers of host-guest functionalization by introducing a first multivalent guest structure, the guest functional groups of which binds non-covalently to the host functional groups present in the multivalent host structure of the first layer of functionalization. The host functional groups present in the first layer of functionalization can also for be used for sensing or purification of the functionalized cell by letting it interact with a surfaced that has been functionalized with complementary guest molecules or structures. Finally, the host functional groups present in the first layer of functionalization can also be used for attaching one or more functional end-groups that are linked to a second guest molecule.

In one embodiment of the present invention, a functionalized in vitro cell comprising a first layer of functionalization further comprises one or more further layers of functionalization, wherein subsequent layers of functionalization are formed by alternating layers of host and guest structures which are non-covalently bound to one another. The one or more further layers for host and guest structures are non-covalently bound to one another via their respective host and guest functional groups.

In one embodiment, a host functional group of the first multivalent host structure of a functionalized in vitro cell according to the invention, is non-covalently bound to a guest functional group of a first multivalent guest structure, wherein the first multivalent guest structure forms a second layer of functionalization.

In yet a further embodiment, a guest functional group of the first multivalent guest structure of a functionalized in vitro cell according to the invention, is non-covalently bound to a host functional group of a second multivalent host structure, wherein the second multivalent host structure forms a third layer of functionalization.

### First Multivalent Guest Structure

Once a cell according to the present invention has been functionalized with a first layer of functionalization, it may be further functionalized by introducing a second layer of functionalization. This second layer is comprised of the first multivalent guest structure, comprising at least two guest functional groups. In one embodiment, first multivalent guest structure comprises a scaffold molecule, which may be selected from the group consisting of poly(isobutylene-alt-maleic anhydride) (PIBMA), PAMAM, poly-acrylic acid, polysaccharides, polypeptides and oligopeptides. In another embodiment, the scaffold molecule is a polypeptide comprising less than about 30, such as, e.g. less than about 25, less than about 20, less than about 15, less than about 10, less than about 5 or less than about 6 amino acids. It may also be an oligo peptide such as, e.g. a dipeptide, a tripeptide, a tetrapeptide or a pentapeptide. In one embodiment the repeat unit of the poly or oligepeptide is β-alanine.

### Functional End-Groups

A “functional end-group” as used herein among others relates to a targeting group, which is a molecule or a part thereof that is either already present on the cell (e.g. surface receptor or enzyme) or can be added to the cell of the present invention to add to or improve the original function of the cell. One possible application is that the functional end-group provides an artificial affinity for other cells (chemotaxis), which relates to the interaction between functionalized cells and their surrounding as is induced by the addition of the components disclosed herein. Targets can be e.g. VEGF, integrins, CXCR4, SDF-1, lectins, polysaccharides, PSMA, endoglin, transporter channels, myelin, P0, Her2, CD40, MMPs, Interleukins, selectins, cytokines, amyloid. The added components (host/guest/targeting end-group) alter the interaction between cells, preferably leading to a stronger interaction between cells. The artificially induced interaction may exist between modified (generally the cells according to the present invention) and tissue expressed proteins or non-modified cells (for instance inflamed tissue that exist in vivo and that need to be targeted). This will be preferred in applications such as cell targeting and/or cell therapy, or between two modified cells (which would be preferred in an application such as tissue engineering). Preferred functional end-groups according to the invention are imaging labels, targeting groups, therapeutic groups, nanoparticles or cloaking groups. Cloaking groups can be used as functional end-groups to coat the cell. Cloaking relates to the prevention of interactions with the immune system. Negatively charged compounds as well as steric (bulky) compound will shield cells from immune cells. An interaction of the immune system with introduced (therapeutic) cells can lead to severe side-effects (such as rejection of the introduced cells). Therefore interaction should be avoided as much as possible, which can be achieved by the methods and means of the present invention. Imaging labels are molecules or parts thereof that can be used in the visualization of molecular features of cells or tissue (molecular imaging). Examples of imaging labels include fluorophores for optical imaging, radioisotopes for nuclear imaging or non-radioactive isotopes for MRI or mass spectrometry imaging. In any event, the functional end-groups allow the cell to change or improve its original function. Functional end-groups are usually introduced in the final component of the layer-building process; after which there is usually no further possibility to build extra layers. Next to a singular functional end-group, a mixture of functional end-groups may also be applied, depending on the application. The functional end-groups can be co-valently attached to the scaffold structure of a multivalent guest or host structure or they can be linked to a host or guest molecule. In the latter case, they interact with a complementary host or guest functional group in the outer layer of guest or host functionalization. Functional end-groups according to the present invention can suitably be selected from the group consisting of an imaging label, a targeting group, a therapeutic group, a nanoparticle, an organic surface, an inorganic surface, a surface of another cell, or a cloaking group. When the target of the functionalized cell according to the present invention is another cell, suitable targeting groups include molecules or parts thereof, which are capable of binding to a cell surface biomarker present on the surface of another cell. For many applications, it is preferred to introduce one or more types of functional end-groups to the cell surface, such as for example a combination of one or more targeting groups with one or more cloaking groups.

In one embodiment the present invention relates to a functionalized in vitro cell wherein the outer layer of functionalization is formed by a multivalent host or guest structure, hereinafter referred to as outer layer of host-guest functionalization, is further functionalized with one or more functional end-groups. The functional end-groups may either be covalently attached to the scaffold structure of the multivalent host or guest structure in the outer layer of host-guest functionalization or may be non-covalently bound to the host or guest functional groups in the outer layer of host-guest functionalization. The latter is conveniently achieved by linking the functional end-group of choice to a host or guest molecule complementary to the host or guest functional groups in the outer layer of host-guest functionalization.

Accordingly, in one embodiment of the present invention the interactions between host or guest functional groups in the outer layer of host-guest functionalization and the functional end-groups are non-covalent. In one embodiment this means that when the outer layer of host-guest functionalization comprises multivalent guest structures, a guest functional group, which is present in the outer layer is non-covalently bound to a host molecule that is linked to a functional end-group. In a further embodiment the host molecule that is linked to a functional end-group is multivalent. And vice versa, when the outer layer of host-guest functionalization comprises multivalent host structures, this means that a host functional group which is present in the outer layer is non-covalently bound to a second guest molecule that is linked to a functional end-group. In a further embodiment the second guest molecule that is linked to a functional end-group is multivalent.

In one embodiment, the first multivalent host structure is further functionalized with one or more functional end-groups. This may be achieved by that a host functional group, which is present in said first multivalent host structure is non-covalently bound to a second guest molecule that is linked to a functional end-group. In a further embodiment the second guest molecule that is linked to a functional end-group is multivalent. Normally, the functionalization with functional end-groups means that the cell is not further functionalized by subsequent layers of host-guest functionalization.

In another embodiment, first multivalent guest structure of the the second layer of functionalization is further functionalized with one or more functional end-groups. This may be achieved by that a guest functional group, which is present in said first multivalent guest structure is non-covalently bound to a host molecule that is linked to a functional end-group. In a further embodiment the host molecule that is linked to a functional end-group is multivalent. Normally, the functionalization with functional end-groups means that the cell is not further functionalized by subsequent layers of host-guest functionalization.

In yet another embodiment, the second multivalent host structure is further functionalized with one or more functional end-groups. This may be achieved by that a host functional group, which is present in second multivalent host structure is non-covalently bound to a second guest molecule that is linked to a functional end-group.

Normally, the functionalization with functional end-groups means that the cell is not further functionalized by subsequent layers of host-guest functionalization.

### Definitions

As used herein, the term “pre-target layer of functionalization” means the layer comprised of one or more guest-modified ligands, such as a plurality of guest-modified ligands, wherein the ligand has specificity for the cell surface biomarker of choice.

As used herein, the term “first layer of functionalization” means the layer comprised of one or more multivalent host structures, such as a plurality of host structures, which are non-covalently bound to the first guest molecules forming part of pre-target layer of functionalization via their respective host and guest functional groups.

As used herein, the term “second layer of functionalization” means the layer comprised of the one or more multivalent second guest structure, such as a plurality of guest structures, which are non-covalently bound to the first multivalent host structures forming first layer of functionalization, via their respective host and guest functional groups.

As used herein, the term “third layer of functionalization” means the layer comprised of the one or more second multivalent guest structures, such as a plurality of guest structures, which are non-covalently bound to the second multivalent host structures forming part of the second layer of functionalization, via their respective host and guest functional groups.

As used herein, the term “outer layer of host-guest functionalization” refers to the last layer of host or guest structures applied to the surface of the cell before applying any functional end-group. In other words, this is the host or guest layer farthest away from the cell. In some embodiments of the present invention, the outer layer of host-guest functionalization constitutes the functionalized surface of the cell and is thereby the same as the “outer layer of functionalization”.

As used herein, the term “outer layer of functionalization” refers to the last layer of functional groups applied to the surface of the cell. In some embodiments of the present invention, a functional end-group is attached to the outer layer of host-guest functionalization, whereby the one or more functional end-groups constitute the functionalized surface of the cell. If no such functional end-group is attached to the outer layer of host-guest functionalization, then this layer is in itself the outer layer of functionalization.

As used herein, the term “functional end-group” refers to the one or more functional groups which can be linked to the outer layer of host or guest structures applied to the surface of the cell.

Using the supramolecular functionalization of the cell surface following the present invention, a great variety of functional end-groups may be introduced on the cell surface. These functional end-groups then dictate the application of this supramolecular concept. Functional end-groups can also consist of inorganic, organic or biological entities and may be introduced after every step of the layer-by-layer functionalization (FIG. 1, top row). Multivalent guest-host interactions are commonly exploited in well-defined systems using gold or silica surfaces. The present invention is the first example that shows that such chemistry is also possible on the surface of living cells, opening up a wide variety of applications (imaging, sensing, targeting and therapy) using the cells that are obtainable by the methods of the present invention.

An important aspect of the technology of the present invention is that the cells can stay viable, and that the cell itself is not changed as the performed functionalization takes place by virtue of non-covalent and reversible interactions on the surface of the cell. Accordingly, the functionalized cells according to the present invention are not covalently or genetically altered, nor has the cell membrane itself been altered. Hence, no genomic modification is taking place; in principle only the outside of the cell is engineered in a reversible manner.

Another important aspect of the methods of the present invention is that the interaction between the biomarker-specific ligand and the biomarker, as well as the interaction between guest and host molecules or functional groups thereof is reversible and non-covalent. This means that the cell surface is coated with layers for a limited period of time. The layers—if such is preferred—may be actively competed away from the surface or will simply be removed in time. This is especially beneficial in settings in which functionalized cells are subsequently used in vivo, for instance when the functionalized cells are (re)introduced into the body, for example for imaging, homing, targeting, chemotaxis, cell therapy, etc. The functionalized cells that are produced according to the present invention will—in vivo—eventually return to their original state because the guest-host and scaffolding will be washed off over the course of time.

### Method of Functionalization

As disclosed herein, in methods of the present invention, cell surfaces are preferably functionalized in aqueous conditions that allow the proper maintenance of the cells, preferably in culturing conditions and using high affinity targeting moieties (ligands). The same process could, however, also be applied in vivo, in industrial- or (waste-) water streams, or in body fluids. The skilled person is aware of the variety of ligands that exists that may be used to target a biomarker of choice. Such ligands may include, but are not limited to, small molecules, peptides, proteins, nanobodies, hormones, and (nano) particles. These high affinity targeting ligand moieties are functionalized (engineered) with one or more guest functional groups.

### Applications of the Functionalized Cells According to the Present Invention.

The cells that are obtained may be used, for instance, for the introduction of functionalities e.g. for therapeutic (introduction of drugs) or imaging purposes (tracking of cells in vitro, or in vivo), for targeted delivery of the cells or their contents (artificial chemotaxis), and for sensing and selection of cells in a mixed population or solution. The sensing may take place using cell-chip interactions enabling multiple goals such as diagnosis (e.g. selecting tumor cells in a mixed cell population) or purification (e.g. extracting cells from industrial or waste water, or extracting cells from body fluids such as blood, plasma, serum, or (breast) milk).

### Applications

Uniquely, the same host-guest interactions can also be used to bind functionalized cells to for instance surfaces such as, but not limited to, functionalized silica, gold, plastic, or other cell membranes (or cells) that are functionalized with matching host or guest groups (FIG. 1, bottom row).

### Applications

Multiple applications are envisioned by the inventors of the present invention. Although the basic technology is the same in each case, the applications cover quite distinct fields. A number of such—non-limiting—applications (imaging, targeted cell delivery/immune therapy, sensing/purification, tissue engineering and drug delivery) are discussed in more detail below.

### Imaging

For staining of cells in diagnostic settings, for (in vivo) tracking of cells and staining of whole tissues, multiple techniques are available in the art. Imaging through the use of ligand-receptor binding is common and has been demonstrated in many forms (Willmann et al. Molecular imaging in drug development Nature Reviews Drug Discovery 7, 591-607, Byrne et al, Active targeting schemes for nanoparticle systems in cancer therapeutics Adv Drug Deliv Rev 60 (15) 1615-1626). However, such techniques are limited when receptors are targeted that only have a low abundance on the cell membrane, or when high amounts of imaging labels are required for proper contrast, e.g. for MRI or immunohistochemistry. The present invention provides a solution to these problems because it enables one to ‘build’ several layers with an increasing number of host or guest functional groups and/or functional end-groups thereby amplifying the number of functional groups exposed on the surface compared to the number of cell surface biomarkers targeted with the ligand-first guest structure, e.g. by using multivalent host structures that in turn allows the binding of more guest functional groups, followed by even more host structures, etc. So, even when the abundance of a particular type of receptor on a particular cell is relatively low (yet high enough to allow multivalent binding), the amount of imaging molecules can become far greater with the methods of the present invention. This concept provides a low-cost alternative for the concept of secondary antibody staining, which is currently being applied in immunohistochemistry. More importantly, it expands the concept to a use in/on living cells. It is clear that the introduction of imaging labels on (living) cells may be used for many different purposes such as cell tracking, tissue staining, histology-based staining of tissues, in vitro or in vivo imaging, or monitoring of receptor internalization processes. The skilled person is aware of the great number of imaging labels that is available in the art, and many of such imaging labels can be used in the methods of the present invention. Examples of suitable imaging labels are (photo)-luminescent labels, (nano)-particles, (e.g. quantum dots, gold particles, or viral nanoparticles), radioactive isotopes, isotopes for mass spectrometry, or CT- and MRI-contrast imaging.

### Targeted Cell Delivery/Immune Therapy

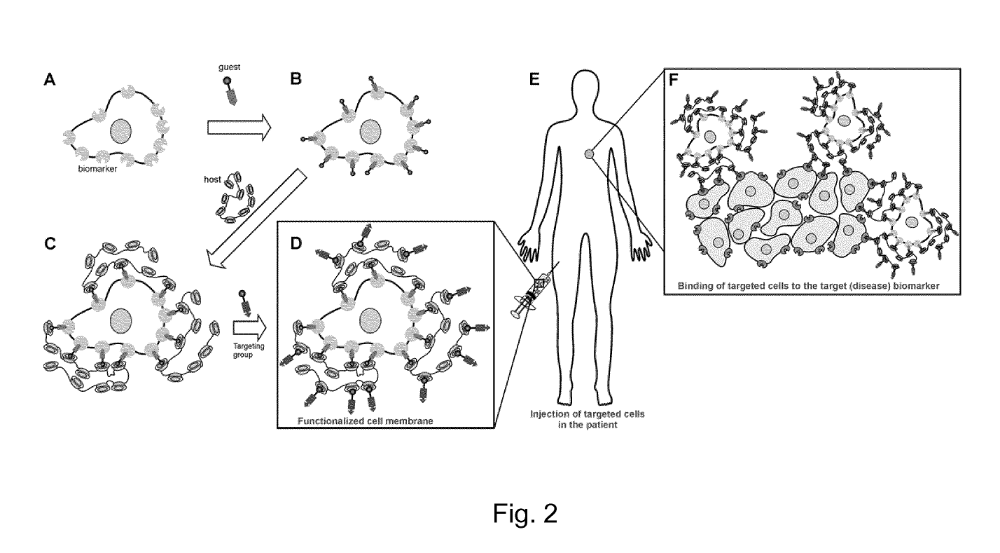

The supramolecular introduction of specific (biomarker) targeting moieties (targeting end-groups) may also provide functional end-groups in the methods of the present invention. These targeting moieties will as a consequence thereof surround the cell membrane. In this embodiment of the invention, the cell surface of a cell of choice is essentially tailored towards interaction with other entities such as cells, tissues, particles, surfaces (or parts thereof). This essentially means that the cell of interest, with its functionalized cell surface, is transformed into a particle that can be targeted to a preferred location. Such transformation of non-targeted cells into a targeting particle may be achieved by using different types of targeting moieties, that may be, but that are not limited to, inhibitors, peptides, nanobodies, proteins (or parts thereof), antibodies (or parts thereof), or even complete receptors (or their binding domains). This type of functionalization provides an artificially induced control over the distribution, chemotaxis and accumulation of cells. FIG. 2 shows the basic concept of this targeted cell delivery embodiment. Basically, as outlined above, cells are incubated with a ligand-guest structure recognizing a specific receptor in the first step. In the second step the cells are incubated with the multivalent host structure, and in a third step the cells are then incubated with guest containing targeting end-groups that bind to the layer of multivalent host structures. In vitro or upon introduction into the body (either locally or in another setting, for instance by intravenous administration) the biomarker affinity of the targeting end-groups causes the functionalized cells to home to the place of interest (cells or tissues for which the affinity has been introduced). Adherence of functionalized cells to their target (in vivo) is preferably quick (<24 h), given the reversible nature of the interactions. This may be promoted using a local delivery (e.g. injection) of the targeting cells. It may also take place by a form of chemotaxis, a natural process wherein a biomarker gradient causes e.g. stem cells to travel over some distance and finally reach the destination where they attach. After local delivery the functionalized cells can perform their function and the supramolecular layers may or may not be washed off over time—either induced and more rapid by an external trigger or simply because of the fact that that the guest-host interaction is non-covalent.

The different applications using targeting cell delivery following the methods of the present invention cover multiple fields of interest. One application of targeted cell delivery lies in the delivery of mesenchymal stem (like) cells in wound healing or to the myocardium to obtain healing of tissue after myocardial infarction. Currently, the local application of such cells is often quite ineffective due to shunting and simple diffusion of the injected cells. A technique that would improve the retention of stem cells in the diseased tissue areas and specifically the retention to the targeted cells is expected to improve the effectiveness of such targeted cell therapies, especially when migration of cells towards the area most in need of a therapeutic function is achieved. The present invention provides the tools to obtain cell retention in such diseased areas, because the specific end-group selection provides for a selected cell-cell or cell-tissue interaction that could not be accomplished when the cell surface of the stem cell is not functionalized. Since the supramolecular layer-by-layer system is reversible due to the non-covalent character of the guest-host and end-group interactions, the technique acts as temporary glue. The cells are in principle ‘normal’ and can therefore function in the most optimal way, whereas cells that would be covered with covalent and non-reversible scaffolds are likely to have been altered in such a way that their natural function may have become impaired. Another interesting application in which the methods of the present invention can be used is the delivery of pancreatic islet cells to the pancreas in diabetes treatment. If desired, trigger mechanisms can be introduced in the coating that can trigger the dissociation of the layers either by activation mechanisms from the local environment e.g. enzymes or pH or by external means such as heat or UV light (Ochs et al. Dopamine-mediated continuous assembly of biodegradable capsules. Chem Mater 2011:3141-3143; Kloxin et al. Photodegradable hydrogels for dynamic tuning of physical and chemical properties. Science 2009 324 (5923):59-63).

In yet another embodiment, the present invention relates to a method in which cells are functionalized and then (upon administration) targeted to areas where cells are needed, and wherein the targeted cells produce/release therapeutic compounds (simple because they were chosen because of that property), or wherein the cells themselves are therapeutic to the area of disease (Orive et al. Encapsulated cell technology: from research to market. Trends in biotechnology 2002 (20)9:382-387). When the methods of the present invention are applied to immune cells, the chemical modifications of the present invention can be used to generate a disease-specific therapeutic immune response by artificially modified immune cells. Example applications wherein biomarker targeted immune cells may be beneficial are: the treatment of tumors or (multidrug resistant) infections. By using the methods of the present invention, one is now able to alter and modify the cell surface of the patient's own immune cells without changing the genetic composition of such cells, without having to train the cells for a immune response, and without changing their therapeutic function. This is of special interest when an immune response towards a complex disease is required and the targeted cells should function in the most optimal way possible. This may for instance be achieved by shunting the blood stream of a patient, and selectively modify only the immune cells, and apply methods to redirect them to the diseased location (by chemotaxis).

Next to targeting and the induction of interaction, these interactions may also be prevented by using the cells and methods of the present invention. Interactions of introduced (targeted) cells with the immune system should be avoided as much as possible to prevent undesired side effect, such as rejection of the introduced cells. For this, so-called cloaking groups (e.g. poly ethylene glycol and derivatives) can be used in the functionalization methods according to the present invention. A combination of cloaking and targeting would be most useful for most applications to move the functional cells to their location. In a similar application, immune cells might be covered with some cloaking groups as a form of immunosuppressive drug. This may prevent interactions with other cells, and this can for instance be useful to temporarily (because of the reversibility) shut down the immune system to prevent rejection of an implant. The use of this temporary and mild immunosuppression effect may even be used for autoimmune diseases. The multivalent scaffold may by itself already perform the functions of a cloaking group, such as PIBMA described in the examples.

### Drug Delivery

The methods of the present invention may also be used to generate functionalized cells wherein the multivalent host structure carries or has an affinity for therapeutic drugs. Although many different types of drug delivery systems, such as liposomes, microspheres and nanoparticles are known in the art, the present invention uses living cells for the delivery of drugs that are kept on the outside of the cells in the scaffold of the layer-by-layer guest-host supramolecular scaffold that is reversibly attached to the cell used for the drug delivery.

Instead of introducing surface modification to healthy cell as a delivery vehicle, the diseased cells may also be functionalized in vivo. After modification, these diseased cells may have an increased affinity for (complementary modified) drugs. In another embodiment, the diseased cells may be coated with multiple layers of multivalent compounds, thereby forming a tough ‘shell’ around these cells. This may for instance be used to prevent outgrowth of infection or prevent metastases of tumors.

Sensing/Removal of Cells from Solution

In the case of sensing, it is now possible to separate cells from a mixed cell population. This is particularly useful when bacteria need to be removed from a solution. In that particular aspect, a receptor on the bacterial cell surface is selected. Multiple host-guest combinations can be used orthogonally, allowing for combined sensing of different cell types in a single procedure.

The methods of the present application can also be used in sensor technologies designed to detect specific cells in solutions, such as body fluids (e.g. blood) or waste streams. Via the same supramolecular design as described above (FIG. 1), cells as a whole can be functionalized through their specific receptors on their cell surface in such a way that they obtain an affinity for a complementary host or guest functionalized surface that may be of any suitable inorganic or organic material, such as silica, gold, metal, plastic, or even plant or tissue material. Like described above for the other applications, specificity is introduced with the first functionalization step (binding of a binding ligand, or a binding domain thereof, preferably a synthetic peptide, that is linked to a guest entity) of the cells the sensor is designed for. Here the embodiment of the invention can be used to determine the relative abundance of cells that are positive for a certain biomarker, mixtures of biomarkers, or a number of different biomarkers. When fully functional, a sensor design can be applied in a variety of applications that all use the same guest-host interactions.

Combining this property with complementary types of host-guest chemistry means the technology can be applied in the development of sensors that detect differently functionalized agents. Simultaneous application of different guest-host interactions can be exploited to generate sensors that give multi-parameter readouts, potentially for different types of cells. The surface modification required for such a sensor is straightforward, ensuring simple integration of this concept in for instance a so-called lab-on-a-chip analysis system. Combinations of any of these applications may also be used.

Next to sensing the cells in solution, the interaction between functionalized cells and surfaces may be used for purification. The methods of the invention can be applied to functionalize cells in a mixed population of cells, or in a solution/body fluid, to purify cells from a solution for further downstream purposes. The binding of the functionalized cells to the functionalized (and compatible) surface may then be used to selectively remove cells (e.g. bacteria, tumor cells) from an aqueous solution such as body fluids (blood, milk, etc.) or (industrial) water streams. The removal of cells through the methods of the invention is also useful in industrial water or waste water treatment, for example in order to remove bacteria from a polluted stream or to recycle bacteria or algae in an aqueous production process. Hence, the invention also applies to functionalizing non-eukaryotic and plant cells, such as bacteria and algae, as long as there are biomarkers that can be utilized to build the guest-host layers on the cell surface. In another application, the binding cells can be purified from the solution. This is for instance a possible way to obtain purified stem cells from blood or from a cell mixture. The great advantage of the functionalized cells of the present invention is that such cells are kept alive and can return to their natural state, as the functionalization is reversible.

### Tissue Engineering

Since the functionalization of the cells through their specific receptor content can be utilized to force specific interactions between cells and a pre-selected material e.g. surface or cells/tissue, one further application of the methods of the present invention is found in tissue engineering. By using a predetermined shape or area, and by functionalizing the engineering surface—this can be a material template or surface, but can even be cells or tissues—in such a way that host-guest compatible functionalized cells of interest bind to the form or area, the layer-by-layer scaffold on the cells can be further used to build layers of the same type of cells or of different types of cells. This technology resembles a type of 3D tissue printing wherein any type of cells in any form or shape should potentially be possible (Matsusaki et al. Three-Dimensional Human Tissue Chips Fabricated by Rapid and Automatic Inkjet Cell Printing. Adv Healthc Mater 2013 (4):534-539). Simple applications are for instance the engineering of skin, wherein functionalized skin cells or stem cells are attracted to a predetermined area on a surface and allowed to grow through the attraction of additional cells that eventually can grow to an in vitro generated piece of skin. Other tissues are of course also feasible and interesting examples are heart muscle tissue, bone, etc.

With the present invention it is now possible to use cell membrane receptors to provide a suitable platform for generic multivalent host-guest interactions on the outer cell surface. With the approach as disclosed herein, the primary labeling agent (the ligand) has to be specific for the receptor that is applied, but all the following steps are generic and can be used for different types of biomarkers and thus the technology can be used to induce an interaction between different types of cells that express different types of biomarkers.

Advantages of the supramolecular layer-by-layer cell-functionalization approach in contrast to techniques used in the art for direct labeling are that all the non-covalent modifications of the invention are reversible. The layer formation and the number of introduced functional groups and their interactions can be optimized for instance by using multiple layers, depending on the desired application. Moreover, complementary generic libraries of multivalent host- and guest-moieties can be generated. Using the methods of the present invention, a variety of functional end-groups can be used for cell tracking (a diagnostic end-group) or targeted cell delivery applications (targeting end-group, which may be an antibody or a specific binding part thereof). When cells are functionalized with the first multivalent host structure layer and onwards, the cells are cloaked, reducing undesired immune responses. The amount and type of functional end-groups can be tuned to the application. A combination of different modalities can be mixed and matched for each application. Due to the generic nature of the layer-by-layer scaffolds, imaging labels can be applied that are not directly compatible with the ligand that interacts with the receptor, thereby enabling one to select any kind of label for any kind of receptor that binds to a ligand that is linked to a suitable guest molecule.

It should be noted that the above-mentioned embodiments illustrate rather than limit the invention, and that those skilled in the art will be able to design many alternative embodiments. As used herein, the verb “comprise” and its conjugations does not exclude the presence of elements or steps other than those stated in a claim. The article “a” or “an” preceding an element does not exclude the presence of a plurality of such elements.

The present invention relates to a functionalized in vitro cell comprising a cell surface biomarker bound to a ligand, wherein said ligand is linked to a first guest molecule, and wherein said first guest molecule is non-covalently bound to a host functional group that is part of a first multivalent host structure, wherein the first multivalent host structure forms a first layer of functionalization.

Said cells may be maintained, or cultured in vitro, but may also be present in vivo. For many purposes (as disclosed herein) the cells of the invention are preferably in vitro cells. The cell surface biomarker that is present on the cell determines the selection of the cells and the ligand that is applied interacts with that biomarker of choice. The interaction between the cell surface biomarker and the ligand is non-covalent and reversible. Moreover, in a preferred aspect, the interaction between the biomarker and the ligand resembles the natural interaction between the biomarker of choice and its ligand. In a highly preferred embodiment, said biomarker is a cell surface receptor. The cell surface receptor makes that a certain cell is selected for building the guest-host layers as disclosed herein. In certain preferred aspects, the multivalent host structure moiety comprises a functional end-group (such as a fluorescent dye, e.g. a fluorescent polymer), which is especially suitable for imaging purposes.

In a preferred aspect, the invention relates to a cell according to the invention, wherein a host functional group of the first multivalent host structure is non-covalently bound to a guest functional group of a first multivalent guest structure, wherein the first multivalent guest structure forms a second layer of functionalization.

In line with the possible presence of a fluorescent dye, such as a fluorescent polymer, in the first multivalent host structure, in another preferred embodiment, said first multivalent guest structure comprises a functional end-group (such as a fluorescent dye, e.g. a fluorescent polymer), which is especially suitable for imaging purposes.

In a further preferred embodiment, a guest functional group, which is present in said first multivalent guest structure is non-covalently bound to a host molecule that is linked to a functional end-group.

The invention also relates to a cell, preferably an in vitro cell according to the invention, wherein the first multivalent host structure is further functionalized with one or more functional end-groups. This functionalization is preferably achieved by a host functional group, which is present in said first multivalent host structure being non-covalently bound to a second guest molecule that is linked to a functional end-group. The different types of cells that can be obtained by the methods of the present invention are schematically depicted in FIG. 1.

In a preferred aspect, the functional end-group as used herein, is an imaging label (such as a fluorescent moiety), a targeting group (such as an antibody or a part thereof), a therapeutic group, a nanoparticle, an organic surface, an inorganic surface, a biological surface (such as that of another cell), or a cloaking group.

In a highly preferred embodiment, the cell surface biomarker (that determines the choice of the cell that is functionalized) is a receptor that is preferably naturally expressed on, in or at the cell surface of the cell of choice. The presence of a receptor and the abundance thereof on a particular cell surface can be determined using methods that are well-known to the person skilled in the art. For many cells that are known, the presence of certain receptors are known from literature, or can—if needed—be determined for each type of cell.

In one preferred aspect, the receptor is the CXCR4 receptor and said ligand is Ac-TZ14011. It should be understood that this combination, as disclosed herein, provides an example of a receptor of choice and a ligand that is known to interact firmly to that receptor. By no means, this combination is meant to limit the scope of the invention because in principle any suitable biomarker (or receptor) can be selected for any kind of cell selection, and when known, the ligand that interacts good or preferably best to such biomarker can be used to functionalize the cells, following the teaching as disclosed herein. In a preferred embodiment a suitable first guest molecule is adamantane and said first host molecule is a cyclodextrin. The interaction between these examples of guest and host are well known to the person skilled in the art. To build further on the cell surface and to functionalize the cells even further, preferably said second guest molecule is adamantane and said second host molecule is a cyclodextrin.

The present invention also relates to a method for functionalizing cells, comprising the steps of:

a) Maintaining a selection of cells in vitro, wherein said cells are selected based on the presence of a cell surface biomarker of choice on the cell surface of said cells;
b) Incubating said cells with a ligand that is able to bind to said cell surface biomarker, wherein said ligand is linked to a first guest molecule;
c) Allowing the ligand to interact with said cell surface biomarker;
d) Incubating said cells with a first multivalent host structure; and
e) Allowing the first multivalent host structure to non-covalently interact with said first guest molecule; in order to obtain functionalized in vitro cells comprising a first layer of functionalization.

It should be understood that the first multivalent host structure is potentially interacting with more than one first guest molecule, such as with at least two first guest molecules. Maintaining a selection of cells in vitro does not always mean that such cells are cultured (in a culture medium) or that the number of cells need to be increased for further processing. Maintaining means in general that the cells of choice are kept in a suitable medium or in their natural medium or environment to be able to take them to the next step of incubating them with a guest-modified ligand. For instance, when cells need to be functionalized such that they can be recognized in a particular mixture, and for instance purified from that mixture, there is no need to culture such cells, but they should be kept in such condition that allows the functionalization. In waste water or in (breast) milk, cells such as bacterial cells, may be present that are functionalized according to the methods of the present invention such that through the functional end-group they can be detected and/or purified from their environment. This enables the purification of—in this particular setup—of waste water or (breast) milk from which the functionalized cells are then removed. However, if needed, the cells of choice may also be cultured in vitro to increase their number or to keep them viable, or to condition them to allow the further functionalization steps. If the cells are then functionalized and ready, they can for instance be used in targeting, or drug delivery. Examples are the re-introduction of functionalized cells into the (human) body and artificial chemotaxis, wherein the functionalized cells of the present invention ‘home’ to a pre-selected position or tissue in the body. This may be a diseased area, for instance where tissue repair is required. In yet another setup, the cells of the present invention are functionalized in a way that they can no longer be recognized by the immune system. The cells may then cloaked with a cloaking group as one of the one or more functional end-groups. A key aspect of the functionalization methods of the present invention is that the interaction between ligand and biomarker, as well as the interaction between guest and host molecules or the functional groups thereof is reversible. The cells may return to their natural, original state when the guest and host layers have disappeared through natural dissociation.

In a further preferred aspect, the invention relates to a method further comprising the steps of:

f) Incubating the cells resulting of step e) with a first multivalent guest structure; and
g) Allowing said first multivalent guest structure to non-covalently interact with the first multivalent host structure;
in order to obtain functionalized in vitro cells comprising a second layer of functionalization.

In yet another preferred aspect, the invention relates to a method further comprising the steps of:

h) Incubating the cells resulting of step g) with a second multivalent host structure; and
i) Allowing the second multivalent host structure to non-covalently interact with said first multivalent guest structure;
in order to obtain functionalized in vitro cells comprising a third layer of functionalization.

In yet another preferred aspect, the invention relates to a method further comprising the steps of:

j) Incubating the cells resulting from any of steps e) or i) with a second guest molecule that is linked to a functional end-group; and
g) Allowing said second guest molecule to non-covalently interact with said first or second multivalent host structure.

In yet a further embodiment, the present invention relates to a method further comprising the steps of:

h) Incubating the cells resulting of step g) with a host molecule that is linked to a functional end-group; and
i) Allowing said host molecule to non-covalently interact with said first multivalent guest structure.

The incubation steps in the herein described methods of functionalization can suitably be performed in a cell culture medium of choice, such as, e.g. DMEM, or in an aqueous solution in which case the incubation steps may suitably be performed at about 0° C. for about 1 hour.

In yet a further embodiment, the present invention relates to a functionalized in vitro cell obtainable by the methods described herein.

As disclosed in detail herein, the cells can be applied in a wide variety of methods in a wide variety of technical fields, including therapy, purification methodology, imaging, sensing, drug delivery, etc. etc. The basic lies in the reversible character of the guest-host interaction and the multivalence of the guest- and/or host moieties that allow the building of a single or multiple layers of multivalent moieties on top of each other. Because the functionalization may make use of any type of biomarker that is present on the cell surface (that has a ligand as the binding partner), any type of viable cell may be used in the methods of the present invention, including eukaryotic and prokaryotic cells, bacterial cells, yeast cells, human cells, mammalian cells, etc. etc. Hence, the present invention also relates to the use of a cell according to the invention or that is obtainable according to a method of the present invention, in: cell tracking, such as imaging; targeted delivery, such as (artificial) chemotaxis; immune therapy; tissue engineering; sensing; purification; and/or pre-targeted cargo delivery. In this particular setting, cargo delivery preferably means drug delivery or cell delivery.

In yet another embodiment, the invention relates to methods of diagnosing disease, wherein cells of interest (for instance cells that indicate the presence of a particular disease, or indicate a potential risk that a subject has to develop a disease) are functionalized according to methods of the present invention. One example is the functionalization (imaging, detection) of cancer cells in a blood stream or other body fluids to diagnose cancer. The methods of the present invention enable one to detect cells that express biomarkers in, on or at the cell surface by adding multiple layers of guest and host entities and imaging labels that are attached to the biomarker via a ligand. The diagnosis may be performed using different functional end-groups. Examples are imaging labels for colorimetric detection or through radioactive labels.

The invention also relates to methods of purifying cells from a solution such as a waste stream or a body fluid, wherein cells of interest are functionalized according to the methods of the present invention, using a ligand of choice that interacts with a biomarker present on the cells of interest, and wherein the functional end-group is an imaging label or a targeting group allowing the specific selection (and removal) of the functionalized cells from the solution. Examples are removal of bacterial, viruses or yeast cells (that are functionalized) from wastewater or other aqueous fluids. When cells are functionalized while present in a body fluid such as blood or milk, the functionalization steps may either be performed in vivo or in vitro. Clearly, functionalization of cells present in a waste stream is generally performed ex vivo.

In yet another embodiment, the invention relates to methods of targeting cells to a location of interest, for instance in the case of artificial chemotaxis, or cell targeting in vivo, wherein cells are functionalized according to the methods of the present invention and using the chemistry as disclosed herein, followed by a step of allowing the functionalized cells to migrate to the location of choice, that—in the case of cell therapy—is often a location somewhere in the (human or animal) body. With the present invention, such is now feasible as any kind of cell may be selected, depending on the biomarker, and functionalized with any kind of suitable targeting end-group. The advantage of the methods of the present invention is particularly that the functionalized cells will remain intact due to the reversible character of the guest-host interactions. This therefore allows migration of cells and loss of the functionalizing groups after some time, preferably when the cells have reached their destination.

The present invention also relates to a method of targeting selected cells to an organic surface, an inorganic surface or a surface of another cell, said method comprising the steps of:

a) Selecting cells based on the presence of a cell surface biomarker of choice on the cell surface of said selected cells;
b) Optionally maintaining or culturing said selected cells in vitro;
c) Allowing the interaction between said selected cells and a ligand that is able to bind to said cell surface biomarker, wherein said ligand is specific for said cell surface biomarker and is linked to a first guest molecule;
d) Allowing the non-covalent interaction between a first multivalent host structure and said first guest molecule; and either of the following steps:
1) Targeting the cells obtained from step d) to said organic surface, said inorganic surface or said surface of another cell, wherein the surface of said organic surface, said inorganic surface or said surface of another cell comprises guest functional groups; or
2) a) Allowing the interaction between a second guest molecule with said first multivalent host structure, wherein said second guest molecule is linked to a targeting group that can specifically bind to an organic surface, an inorganic surface or a surface of another cell; and b) Targeting the cells obtained from step 2) a) to said organic surface, said inorganic surface or said surface of another cell, wherein the surface of said organic surface, said inorganic surface or said surface of another cell comprises a target of the targeting group.

In another embodiment, the first multivalent host structure of the cells obtained in step d) in the above-mentioned method of targeting are allowed to interact with a first multivalent guest structure, which allows a further build-up of layers, through the interaction of a host molecule that binds to a guest functional group within said multivalent guest structure that is not bound to a host functional group of the first multivalent host structure, and wherein said host molecule is linked to a functional end-group (that is, in the case of targeting cells, preferably a targeting group). In yet another embodiment, the a guest functional group of the first multivalent guest structure or (in the case of a further layer, the host functional group of a second multivalent host structure) is directly linked to an organic surface, an inorganic surface or the surface of another cell, although in the latter case, this would be an interaction that would occur ex vivo, rather than in in vivo cell targeting. In yet another embodiment, if it is desired to target drugs to a predetermined position in the body, the multivalent guest or host structures can be further linked (either through further host or guest interactions) with a drug of choice, which may then reach its destination through the functionalization methods as disclosed herein. In the targeting method described above, it may be desirable to culture the selected cells to increase their number or to condition them for further processing. This is not always required for every targeting purpose. However, and this is also optional, when cells are selected for the presence of a specific biomarker, such cells may be obtained from a human or animal subject, purified by means known to the person skilled in the art, and kept in vitro for a certain amount of time. The cells may then be functionalized as disclosed herein and then used for targeting to the surface of choice. In the case of cellular targeting in vivo, the functionalized cell preferably is then uploaded with a targeting group that is to a certain extent specific for the surface of another cell to which the functionalized cell should ‘home’. One can also envision that targeting is not always to the surface of another cell, but may also be to organic or inorganic surfaces of choice, that may be present in vivo or in vitro, whatever the application may be. Crucial is that the cells are selected for their specific cell surface biomarker, that a ligand is used that is specific for said biomarker and that the building of guest-host layers is through multivalence and that the guest-host interactions are reversible (non-covalent).

The methods and means of the present invention also enable one to build tissue by attracting other cells through the guest-host interactions as disclosed herein. The great advantage in this is that the means that are applied (the guest and host molecules and structures) will be removed as they are non-covalently attached, which therefore results in (healthy) tissue from which the guest and host molecules or structures can be easily removed.

In yet another embodiment, the cells and methods of the present invention can be used in circumventing immune attacks. By using cloaking groups as the functional end-groups, cells can be shielded from the immune system until they reached their destination or until they need to perform their function. Especially when cells are applied that are strange to the body and that need to be administered for a particular purpose, the cloaking groups can (for a limited amount of time) shield the functionalized cells from the immune system and allow the cells to act.

In yet another embodiment, the cells and methods may be used in a pre-targeting fashion. In nuclear imaging and therapy, the pre-targeting fashion delivers one high-affinity compound to the target site. After that, a radioactive compound with high affinity is administered. With the methods of the present invention, a similar tactic may be used; a target (diseased) location may be pre-targeted with either host or guest molecules. After that, a complementary binding partner can be used to deliver cargo to the target site. This cargo might comprise of imaging agents, therapeutic compounds, (therapeutic) cells or whatever needs to be delivered at the target site.

### EXAMPLES

### Example 1. Experimental Procedures and Synthesis of Compounds

The compounds described in this example are the compounds used in examples 2 to 10. All chemicals were obtained from commercial sources and used without further purification. ISOBAM-04 (PIBMA Mw 60.000) was kindly supplied by Kuraray Europe GmbH. NMR spectra were taken by using a Bruker DPX 300 spectrometer (300 MHz 1H NMR) or a Bruker AMX 500-MHz with a TXI gradient probe. All spectra were referenced to residual solvent signal or TMS. HPLC was performed on a Waters system by using a 1525EF pump and a 2489 UV detector. For preparative HPLC a Dr. Maisch, GmbH, Reprosil-Pur 120 C18-AQ 10 μm (250×20 mm) column was used and a gradient of 0.1% TFA in H2O/CH3CN (95:5) to 0.1% TFA in H2O/CH3CN (5:95) in 40 min was employed. For analytical HPLC a Dr. Maisch, GmbH, Reprosil-Pur C18-AQ 5 μm (250×4.6 mm) column was used and a gradient of 0.1% TFA in H2O/CH3CN (95:5) to 0.1% TFA in H2O/CH3CN (5:95) in 40 min was employed. MALDI-ToF measurements were performed on a Bruker Microflex. For dialysis, Spectra/Por® 1 dialysis membrane (MWCO=6-8 kD) or Sigma Pur-A-Lyzer™ Mega 3500 were used.

### Example 1.1. Preparation of Ac-TZ14011-Ad (Ligand Linked to First Guest Molecule)

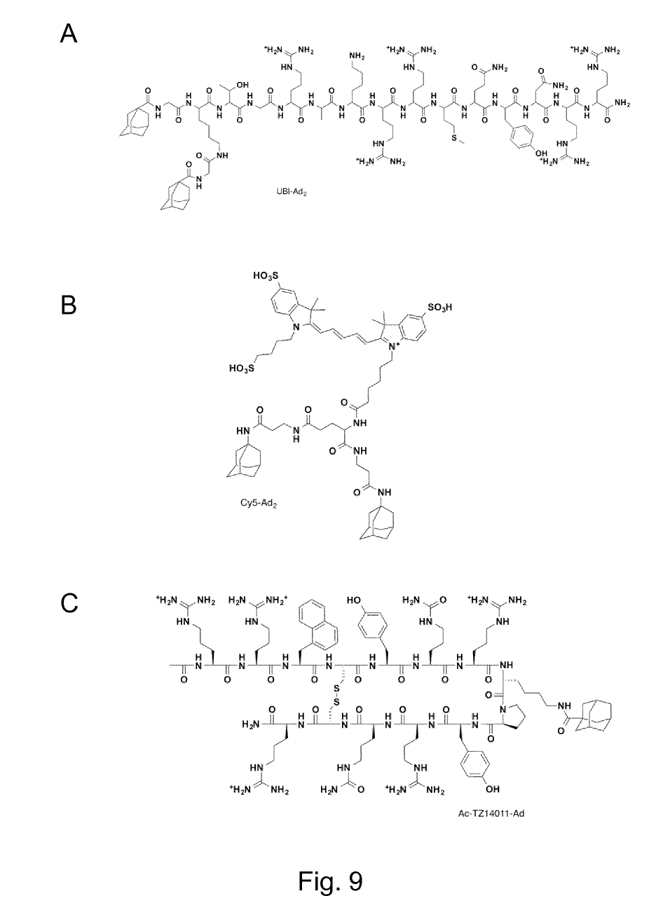

The chemical structure of Ac-TZ14011-Ad is given in FIG. 9C. Benzotriazol-1-yl-oxytripyrrolidinophosphonium hexafluorophosphate (PyBOP) (0.63 mg, 1.2 μmol), 1-adamantanecarboxylic acid (0.54 mg, 3 μmol) and N,N-Diisopropylethylamine (DIPEA) (1.7 μl, 10 μmol) were dissolved in 100 μl dry DMF and stirred for 5 min at RT. This was added to a solution of Ac-TZ14011 (2.8 mg, 1 μmol), synthesized as previously described (Kuil et al. 2011) in 1 ml dry DMF. The reaction mixture was stirred for 2 d at RT, and the product was purified by preparative HPLC. The product fraction was lyophilized to give 1.8 mg (79% yield) of a white powder. MS (MALDI-ToF): [C103H158N35O20S2]+ calcd 2269.2, found 2271.3.

### Example 1.2. Preparation of the Multivalent Host Structure

PIBMA of the suitable length (Mw 6000 or 60.000, 5 μmol or 0.5 μmol) and an amine-functional fluorescent dye (Cy3 or Cy5) (1.1 eq) were dissolved in 3 ml dry dimethyl sulfoxide (DMSO), and DIPEA (50 μl, 250 μmol) was added. This solution was stirred at 80° C. for 7 h, then 6-monodeoxy-6-monoamino-β-cyclodextrin hydrochloride (95 mg, 80 μmol) was added and the solution was stirred for another 3 d at 80° C. After cooling to RT, the polymer was dialyzed against H2O for 1 d. The solution was dialyzed against a pH 9.0 buffer for 1 d and dialyzed against H2O for 5 d while daily refreshing of the dialysis medium. The solution was lyophilized to give a colored powder in 92-93% yield. For non-fluorescent polymers, the synthesis was similar, except that there was no fluorescent dye added in the first step. White powder was obtained in 62-73% yield.

### Example 1.3. Monovalent Cyclodextrin Attached to Cy5

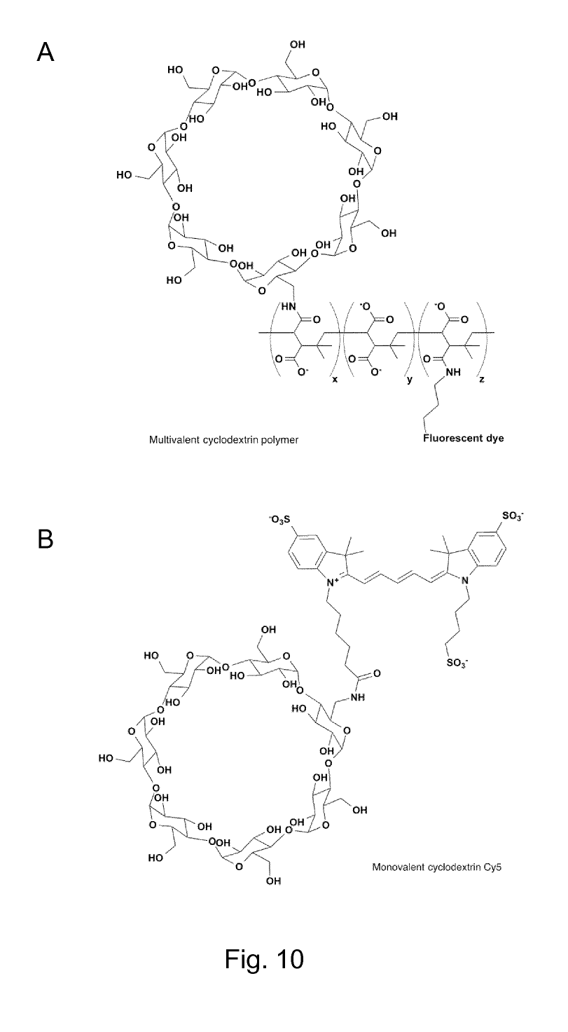

Cy5 was synthesized as previously described (Mujumdar et al. Bioconjug Chem. 1993; 4(2):105-11). Cy5 (4.2 mg, 5 μmol), 6-monodeoxy-6-monoamino-6-cyclodextrin hydrochloride (5.9 mg, 5 μmol) and PyBOP (5.2 mg, 10 μmol) were dissolved in 1 ml dry DMSO. DiPEA (3.5 μl, 20 μmol) was added and the mixture was stirred O/N at RT. The products were precipitated in CH2Cl2 and purified with preparative HPLC. The product fraction was lyophilized to give a blue powder (5.2 mg, 53%). The chemical structure of a monovalent version of a cyclodextrin linked to Cy5 is presented in FIG. 10B.

### Example 1.4. Bivalent Adamantane Attached to Cy5

Boc-Glu-(6-Ala)2 (101 mg, 0.26 mmol), synthesized as described before (J. Kuil et al. Hybrid peptide dendrimers for imaging of chemokine receptor 4 (cxcr4) expression. Molecular pharmaceutics 2011, 8, 2444-2453), PyBOP (676 mg, 1.3 mmol) and DIPEA (530 μl, 3 mmol) were dissolved in 5 ml dry DMF. The solution quickly turned yellow/orange. After 5 min, amantadine hydrochloride (244 mg, 1.3 mmol) was added and the mixture was stirred for 3 days at room temperature. The product was purified by column chromatography (eluens CH2Cl2:MeOH 9:1). The product fractions were collected and the solvents were removed. To the product was added 4 ml 25% TFA in CH2Cl2 and the reaction was stirred overnight. After evaporation of the solvents in vacuo, the product was dissolved in H2O:MeCN and lyophilized to give 111 mg of a white solid (0.17 mmol, 65%).

H-Glu-(6-Ala-Ad)2 (20 mg, 30 μmol) was dissolved in 2 ml dry DMF. Cy5-COOH (25 mg, 30 μmol) and PyBOP (16 mg, 30 μmol) were added. DIPEA (17 μl, 100 μmol) was added and the reaction was stirred at RT overnight. The product was purified by preparative HPLC, the product fractions were collected and lyophilized to give a blue solid (9.0 mg, 23%).

MS (MALDI-ToF): [C66H92N7O14S3]+ calcd 1303.7, found 1303.9. 1H NMR (300 MHz, DMSO-d6): 8.35 (t, 2H, β-H of bridge), 7.80 (s, 2H, H4 of indole), 7.60 (d, 2H, H6 indole), 7.31 (dd, 2H, H7 indole), 6.58 (t, 1H, γ-H of bridge), 6.34 (m, 2H, α-H of bridge), 4.07 (m, 4H, CH2-N), 1.84 (s, 6H, γCH2 Ad), 1.68 (s, 12H, CH3), 1.57 (s, αCH2 Ad). 2.0-1.0 Various peaks of CH2. 1H NMR (300 MHz, D2O): 8.06 (t, 2H), 7.85-7.80 (m, 4H), 7.33 (dd, 2H), 6.62 (t, 1H), 6.3 (t, 2H), 4.17-4.07 (m, 5H, NCH2 & Glu-Ca), 3.36 (m, 4H), 2.92 (t, 2H), 2.29-2.18 (m, 8H), singlets at 1.86, 1.80, 1.65, 1.35.

The chemical structure of a bivalent adamantane attached to Cy5 is presented in FIG. 9B.

### Example 1.5. Bivalent Adamantane Attached to UBI29-41

15 μmol Resin-bound Fmoc-Lys(Fmoc)-Thr(tBu)-Gly-Arg(Pmc)-Ala-Lys(Boc)-Arg(Pmc)-Arg(Pmc)-Met-Gln(Trt)-Tyr(tBu)-Ans(Trt)-Arg(Pmc)-Arg(Pmc) was synthesized by solid-phase peptide synthesis on a Rink amide resin. Standard Fmoc/tBu strategy was used with PyBOP as coupling reagent. After deprotection with 20% piperidine in DMF (2 times 20 min), glycine was coupled to the N-terminus and the lysine side chain by reacting Fmoc-Gly-OH (36 mg, 120 μmol) in the presence of PyBOP (62 mg, 120 μmol), HOBt (16 mg, 120 μmol) and DIPEA (40 μL, 240 μmol) for 2 h. After deprotection with 20% piperidine in DMF (2 times 20 min), 1-adamantanecarbonyl chloride (24 mg, 120 μmol), hydroxybenzotriazole (HOBt) (16 mg, 120 μmol) and DIPEA (40 μL, 240 μmol) were added and the reaction was stirred overnight. After deprotection and cleavage of the resin with TFA:H2O:TIS (95:2.5:2.5), the product was precipitated in ice cold MTBE:Hexanes (1:1), dissolved in water and lyophilized. The crude product (35 mg) was purified by HPLC. After pooling of the pure fractions and lyophilization, 16 mg (36%) of product was obtained as a white fluffy solid.

The chemical structure of a bivalent adamantane attached to UBI29-41 is presented in FIG. 9A.

### Example 1.6. Cell Culture

Human MDA-MB-231 cells, transfected with human CXCR4 conjugated to GFP, were kindly provided by Dr. Gary Luker (Center for Molecular Imaging, University of Michigan, USA) (Song et al., PLoS One 2009, 4, e5756). This cell line was used as CXCR4 positive cell line (MDA-GFP-CXCR4+). Native MDA-MB-231 with low CXCR4 expression was used as a negative control (van den Berg et al., Transl Oncol 2011, 4, 234). MDA-GFP-CXCR4+ and MDA-MB-231 were maintained in Dulbecco's minimum essential medium (DMEM) enriched with 10% fetal bovine serum and 5 mL Penicillin/Streptomycin (10000 units/mL Penicillin; 10000 μg/mL Streptomycin) (all Life Technologies Inc., Breda, The Netherlands). Cells were kept under standard culture conditions (37° C. and 5% CO2).

### Example 1.7. Confocal Imaging

Live cell images were taken on a Leica SP5 confocal microscope under 63× magnification. Nucleus staining was done by incubation with 1 μg/ml Hoechst 33342 for 15 min at 37° C. Hoechst fluorescence was measured using 405 nm excitation and emission 420-470 nm. GFP luminescence (coming from the receptor itself) was measured using 488 nm excitation and emission was collected at 500-525 nm. Cy3 fluorescence was measured using 514 or 561 nm excitation and 570-600 emission. Cy5 fluorescence was measured using excitation at 633 nm and emission was collected at 650-700 nm. Cells were thoroughly washed with PBS before taking confocal microscopy images.

### Example 2. Proof of Concept of Imaging Cells Using a Multivalent Host Structure

The necessity of a multivalent approach was studied by direct comparison between an entity comprising polymeric host functional groups (multivalent) and a monovalent host functional group. A genetically modified MDA-MB-231 human breast cancer cell line that has the Green Fluorescent Protein (GFP) directly coupled to the overexpressed CXCR4 receptor was used to study the interactions with CXCR4 positive cells. The CXCR4 chemokine receptor is over-expressed in the membrane of many human cancer cells, but CXCR4 also play a major role in the chemotaxis of stem cells. The cyclic peptide Ac-TZ14011 is often used to target this receptor and was also chosen here as the ligand. This peptide was modified with an adamantyl group, which represents the first guest molecule. The binding strength between the receptor and the peptide was determined by competition with a previously reported modified Ac-TZ14011 derivative. For this, MDA-GFP-CXCR4+ cells were trypsinized, divided into aliquots containing 300,000 cells, centrifuged (300 g, 3 min, 4° C.) and decanted. For competition experiments, different concentrations ranging between 0.5-15000 nM of Ac-TZ14011-Ad in the presence of a reference agent for CXCR4 targeting, Ac-TZ14011-MSAP (250 nM), in 120 μl of PBS containing 0.1% BSA were added. Cells were incubated for 1 h at 4° C. The cells were then washed two times with 1% BSA in PBS, resuspended in 1% BSA in PBS and fluorescence of the reference compound was measured by using a BD FacsCanto™ II flow cytometer with APC-Cy7 settings. Live cells were gated on forward scatter and side scatter and 10000 viable cells were analyzed. All experiments were performed in duplicate. The normalized means were fitted with equations in the GraphPad Prism 6 software. The KD values were calculated by using the “Binding-Competitive, One site-Fit Ki” nonlinear regression equation of GraphPad Prism. The KD value of Ac-TZ14011-MSAP (186.9 nm) has previously been reported (Kuil et al. 2011). Flow cytometry data gave a KD of 56±9 nM. The effect of the small adamantane functional group (adamantly) on the binding strength of the peptide was very small compared to for instance the effect of a fluorophore on the binding strength.

Two types of multivalent host structures were generated: one ‘short’ with about 9 to 11 cyclodextrin molecules and labeled with Cy5 as the functional imaging group, and the other ‘long’ with about 72 cyclodextrin molecules (ranges between 70 and 82) and labeled with Cy3 as the functional imaging group. Cy3 and Cy5 were chosen as the fluorophores (functional imaging groups) due to the excellent brightness and high water solubility.

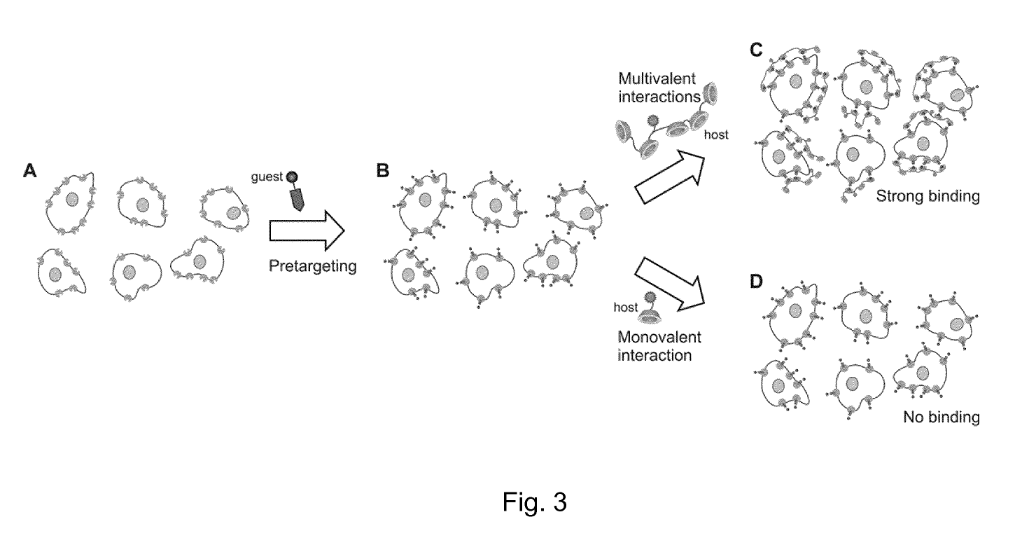

FIG. 3 shows the set-up of the experiment. Cells expressing the CXCR4 receptor that were first incubated with the Ac-TZ14011-Ad ligand as the guest molecule were subsequently incubated with either a multivalent cyclodextrin structure (as the host structure; middle cell in the middle row) or with a monovalent cyclodextrin molecule (leftmost cell in the upper row). Attached to the cyclodextrin was Cy5 as the functional imaging group. For the linkage between the cyclodextrin molecules (to generate a multivalent host structure), the inventors selected poly(isobutylene-alt-maleic anhydride) (PIBMA) as the backbone (also called scaffold polymer). This compound is available in different lengths (6 kD and 60 kD were used in this proof-of-concept study), can be easily grafted and after hydrolysis it is very water soluble. The large amount of negative charges at physiological pH prevents non-specific cellular uptake and decreases interaction with the immune system.

The labeling of cells in this two-step procedure showed that the polymers were bound to the cell membrane and showed good overlap with the GFP signal of the receptor (data not shown). In contrast, when the monovalent cyclodextrin was used, no staining was observed of the Cy5 label. Results obtained with the ±11- and the ±72-containing cyclodextrin functional groups were comparable (data not shown). When monovalent cyclodextrins were used, cells did not stain for Cy5.

To show that the interaction is specific for the CXCR4 receptor, a heterogeneous mixture of CXCR4-positive and -negative cells was grown together in the same dish on glass and incubated together, first for 1 h with the Ac-TZ14011-Ad ligand-guest combination and subsequently for 1 h with the multivalent cyclodextrin Cy5 polymer.

The CXCR4-positive cells were identified based on the GFP signal that was only abundant in the CXCR4-positive cells. The negative cell line was a native MDA-MB-231 line that does express the CXCR4 receptor, but at very low levels. The multivalent two-step labeling showed that the CXCR4-positive cells stained brightly at the cell surface (due to the presence of the Cy5 fluorophore), while the negative cells did not show any or very little staining (data not shown). This strongly indicates that the binding of the cyclodextrins to the cells takes place via the adamantane group that is attached to the ligand bound to the CXCR4 receptor, and that the approach is receptor specific.

Next to the CXCR4-positive breast cancer cell lines that was used in these studies, the experiments were repeated using CXCR4-positive human cardiac stem cells. The obtained results are similar to those obtained with the tumor cell line, which indicates that that is a good model for stem cells.

### Example 3. Stability and Reversibility of the Guest-Host Interaction

The cells generated in Example 2, comprising as a layer a multivalent cyclodextrin polymeric structure (linked to the fluorophore Cy5), wherein the cyclodextrins are attached to the cell surface through adamantane that is linked to the ligand Ac-TZ14011 (Ac-TZ14011-Ad) that is bound to the CXCR4 receptor, were tested in a stability assay. The cells were washed thoroughly and the polymer binding to the cell surface was followed in time. The polymers stay bound to the cells for at least a few hours, even in the presence of excess host (monovalent cyclodextrin molecules) or guest molecules (monovalent adamantane-NH2). Confocal images confirmed that the polymers stay on the membrane of the cells.

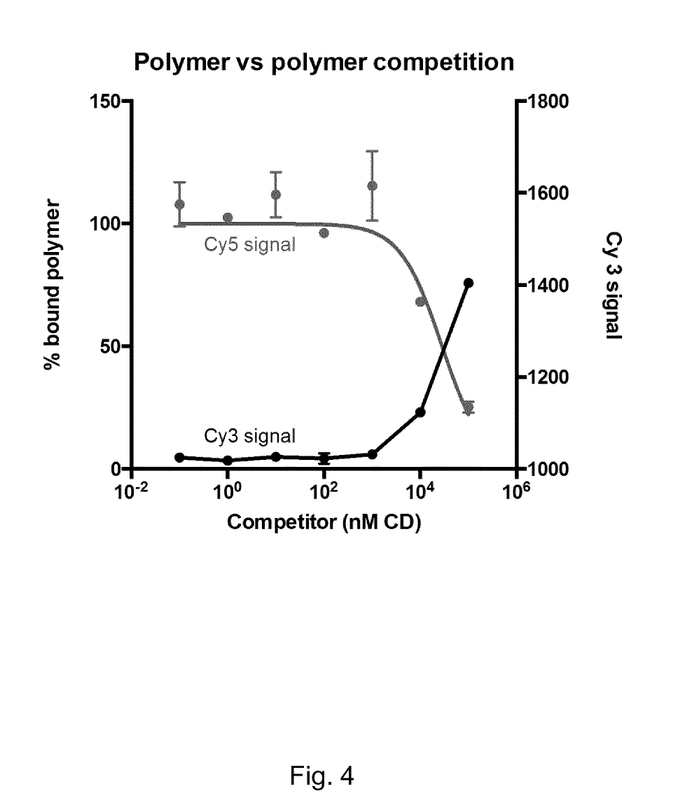

FIG. 4 shows the results when a competitor, in this case a multivalent cyclodextrin Cy3 polymer, was used to compete away the Cy5 labeled polymer. At a concentration of about 104 nM the Cy5 labeled polymer started to be replaced by the Cy3 labeled polymer, showing that the binding of the host to the guest entity is reversible and not permanent. The conclusion is that the polymer binding to the cell surface through the first guest molecule is very strong and stable, yet reversible.

### Example 4. Building a Layer with Quantum Dots (Nanoparticles) as the Host Structure

Next to functionalizing the first guest molecules linked to the ligand binding the marker or receptor in the cell surface with a multivalent polymer, they can also be used to bind other multivalent entities or structures such as dendrimers, functionalized nanoparticles, and functionalized bacteria/cells and even complete functionalized surfaces. In this example, CXCR4-GFP positive breast cancer cells were incubated in a mixture with native MDA-MB-231 cells as in Example 2, and subsequently incubated with Ac-TZ14011-Ad as the ligand-guest entity. To show that the interaction of the first guest molecule to the first multivalent host structure is not dependent on the backbone (such as PIBMA), CdTe quantum dots were covered with cyclodextrin molecules and used for binding to the first guest molecule.

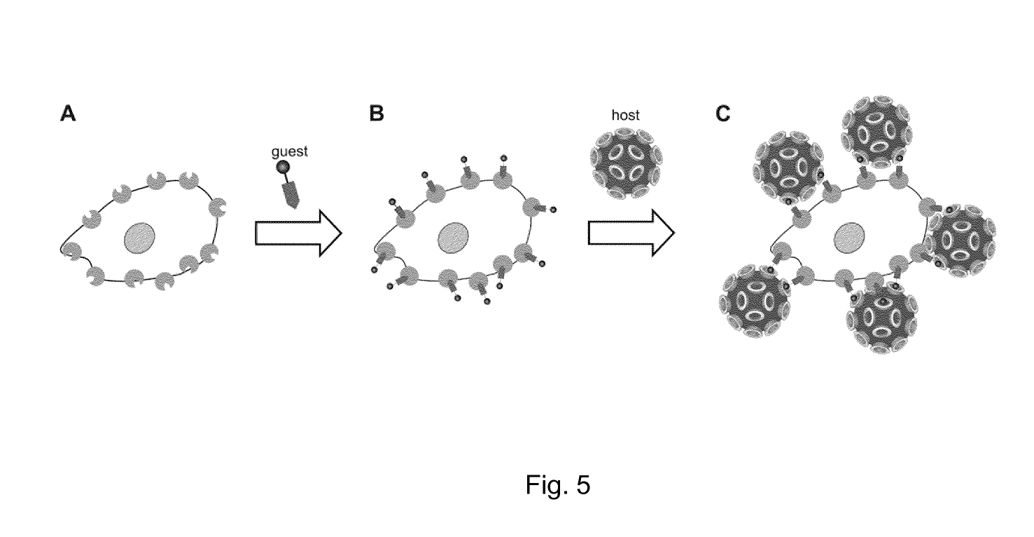

FIG. 5 shows a schematic representation of the quantum dots binding to the first guest molecules, and hence, to the cells. The scale between cells and nanoparticles is not representative, but adjusted for clarity. The staining for GFP (cell surface of the CXCR4 positive cells) and staining for quantum dots strongly overlaps, and that native cells with a low abundance of CXCR4 receptors are hardly stained (data not shown). This shows that when the host structure is presented as a particle, in this case a quantum dot, it can bind specifically to the ligand that is bound to the cell membrane receptor.

### Example 5. Building Multiple Layers

It was further investigated whether it would be possible to generate multiple layers of guest-host structures. Again, as in Example 2, CXCR4 positive breast cancer cells were incubated for 1 h with the ligand-guest Ac-TZ14011-Ad compound and subsequently incubated for 1 h with a Cy3-cyclodextrin multivalent polymer. This was then followed by another incubation for 1 h with an adamantane-Cy5 polymer as the second guest structure and Cy5 as the imaging label. This order of events is represented by the middle row in FIG. 1. Confocal microscopy with coloring of GFP, Cy3 and Cy5 showed clear overlap of staining of all three labels (data not shown), strongly indicating that another layer of a first multivalent guest structure is possible. Continuing this way, confocal microscopy imaging has so far confirmed the successful deposition of up to 4 layers of alternating host- and guest polymers, and there is no indication that this is the maximum.

### Example 6. Attaching a Second Guest Group Attached to a Functional End-Group to a Multivalent Host Structure

It was then investigated whether it would be possible to achieve imaging of cells using a second non-polymeric guest molecule linked to an imaging label. Similar to the examples above, CXCR4-positive cells were grown and incubated for 1 h with the ligand-guest Ac-TZ14011-Ad compound and subsequently incubated for 1 h with the first host structure, in this case a Cy3-labeled multivalent cyclodextrin polymer. Staining of the GFP molecule (indicating the presence of the CXCR4 receptor) clearly overlapped with the presence of the Cy3 label (data not shown). In a next step, a second guest molecule, bivalent adamantane linked to another imaging label, Cy5, was incubated for 1 h. Also the staining of the Cy5 label clearly overlapped the staining of the Cy3 label and GFP, showing that also another functional end-group, attached to a second (non-multivalent) guest molecule can be used in the approach taken by the inventors. It was found that at least two adamantyl-groups in the same molecule were necessary to provide a strong enough interaction with the polymer; a compound where one adamantane was attached to Cy5 showed no staining of the cell membrane.

### Example 7. Sensing of Functionalized Cells

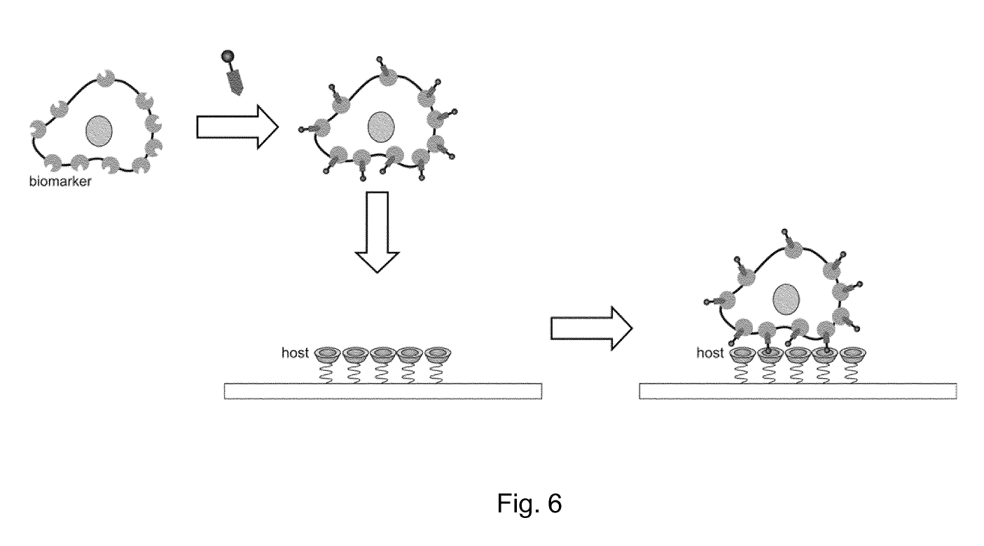

A glass slide was coated with β-cyclodextrin in a line of 400 micrometer wide. CXCR4 expressing cells (see previous examples) were incubated for 1 h with the ligand-guest compound Ac-TZ14011-Ad as discussed above and then added to the glass slide and incubated for 15 min. FIG. 6 shows that the functionalized cells preferentially attach to the area coated with the cyclodextrin molecule (acting as the host). The conclusion of this experiment is that cells can be selectively found (and bound) within a solution by using the guest-host chemistry of the present invention. This enables one to purify (and at least to sense) particular types of cells within a solution and/or mixture of cells.

### Example 8. Functionalizing Bacterial Cells

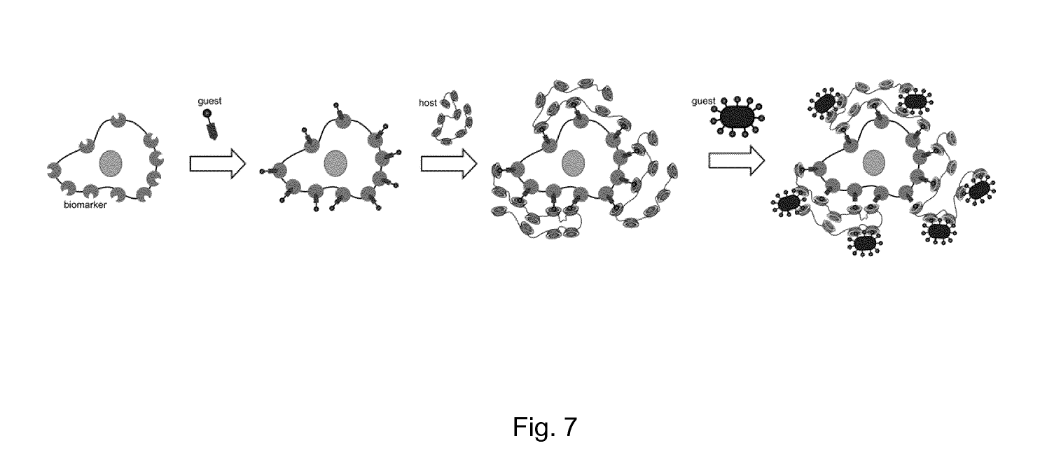

It was also investigated whether the supramolecular chemistry that was applied to mammalian (eukaryotic) cancer cells from humans could also be applied to completely distinct cells, namely bacteria (prokaryotic). For this, CXCR4 positive cells were incubated with the ligand-guest compound Ac-TZ14011-Ad as described in the previous examples. These coated cells were then incubated further with a multivalent first host structure, represented by the polymeric cyclodextrin compound, labeled with the imaging label Cy5, also as described above. Next, these cells were either incubated with wt bacteria, or with bacteria that were coated with adamantane functional groups by having their surfaces functionalized using adamantly-labeled antimicrobial peptides. The adamantane-coated bacteria bound to the cyclodextrins that were still available in the outer layer, whereas the unmodified bacteria did not bind to the cancer cells. FIG. 7 shows a schematic representation of the binding of adamantane-modified bacteria to cyclodextrin-coated cells. Although this is an artificial system, the experiment does show that also bacterial cells can be targeted to other cells through the supramolecular chemistry of the present invention, which enables one to also functionalize bacterial cells for multi-purpose applications, such as waste stream purifications or sensing of contaminations by bacteria.

### Example 9. Targeting Cells to a Predetermined Cell of Interest

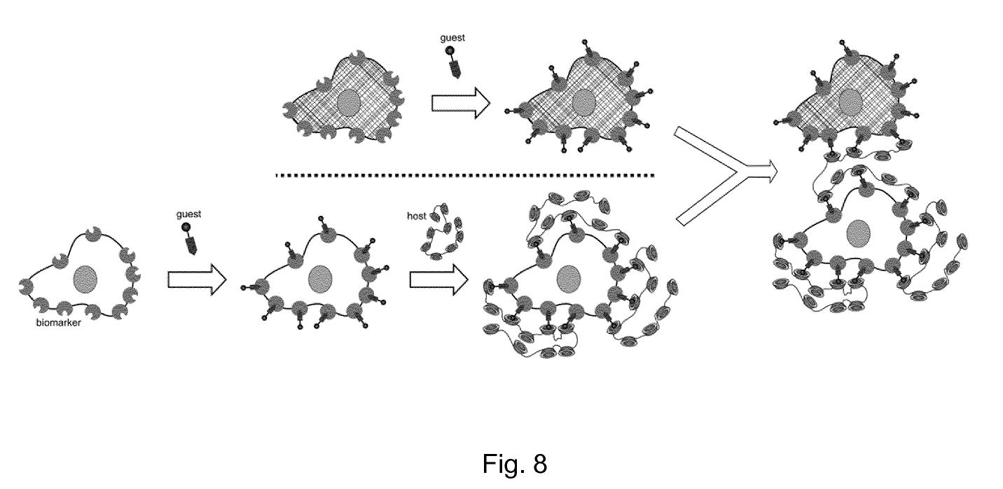

It was also investigated whether the controlled interaction between bacteria and eukaryotic cells (Example 8) could be expanded to the interaction between two eukaryotic cells. Similar to the examples above, CXCR4-positive cells were incubated for 1 h with the ligand-guest Ac-TZ14011-Ad compound and subsequently incubated for 1 h with the first host structure, in this case a Cy3-labeled multivalent cyclodextrin polymer. In a different flask, another batch of CXCR4-positive cells was incubated in suspension with ligand-guest Ac-TZ14011-Ad compound for 1 h. The thus-obtained suspension of adamantane-coated cells was added to microscope slides with the cyclodextrin-polymer coated cells. FIG. 8 depicts a schematic drawing of the steps that were performed in this experiment. Selective binding between the two differently coated cells was achieved (data not shown). This example also indicates that it is feasible to force artificial interactions between cells, for instance when it is preferred to have cells home to a certain destination within a tissue or a living organism. The targeting of stem cells to a diseased area within the body (stem cell transplantation, by applying specific cell-cell interactions) is one of the applications that are within reach by using the cell surface, receptor dependent non-covalent layer-by-layer guest-host chemistry of the present invention.

### Example 10. Protection from External Factors by Polymer Coating

The coating effect provided by the polymers was tested in a model experiment. Similar to the examples above, CXCR4-positive cells were incubated for 1 h with the ligand-guest Ac-TZ14011-Ad compound and subsequently incubated for 1 h with the first host structure, in this case a Cy3-labeled multivalent cyclodextrin polymer. The binding of wt bacteria to these coated cells was measured using a confocal microscope. Image quantification showed that the average amounts of bacteria per cell was halved compared to the binding of wt bacteria to unmodified cells. This examples indicates that the polymer coating provides protection against bacteria, and most likely also to other factors.

### Example 11. Experimental Procedures and Synthesis of Compounds

The compounds described in this example are the compounds used in examples 12 to 22. All chemicals were obtained from commercial sources and used without further purification. ISOBAM-04 was kindly supplied by Kuraray Europe GmbH free of charge. NMR spectra were recorded using a Bruker DPX 300 spectrometer (300 MHz 1H NMR) or a Bruker AMX 500 MHz with a TXI gradient probe and are referenced to residual solvent signal or TMS. HPLC was performed on a Waters system by using a 1525EF pump and a 2489 UV detector. For the MTT assay the Perkin Elmer plate reader 1420 Multilabel Counter was applied. For preparative HPLC a Dr. Maisch GmbH, Reprosil-Pur 120 C18-AQ 10 μm (250×20 mm) column was used and a gradient of 0.1% TFA in H2O/CH3CN (95:5) to 0.1% TFA in H2O/CH3CN (5:95) in 40 min was employed. For analytical HPLC a Dr. Maisch GmbH, Reprosil-Pur C18-AQ 5 μm (250×4.6 mm) column was used and a gradient of 0.1% TFA in H2O/CH3CN (95:5) to 0.1% TFA in H2O/CH3CN (5:95) in 40 min was employed. MALDI-TOF measurements were performed on a Bruker Microflex. For dialysis Sigma Pur-A-Lyzer™ Mega 3500 tubes were used.

### Example 11.1. Synthesis of the Cyanine-Dye Building Blocks

### Example 11.1.1. Indole

The indole-based building blocks; Indole-Sulfonate, Indole-COOH, sulfoindole-Sulfonate and sulfoindole-COOH were synthesized according a previously reported procedure (Bunschoten et al. Bioconjugate Chem in press, DOI: 10.1021/acs.bioconjchem.6b00093; Mujumdar et al., Bioconjugate Chem 1993, 4, 105), while the indole-based building blocks; indole-Phth, Indole-AmineBoc, and sulfoindole-AmineBoc were synthesized according to an adjusted synthesis method based on published procedure (Mujumdar et al., Bioconjugate Chem 1993, 4, 105; Shershov et al., Dyes and Pigments 2013, 97, 353). The crude product of the Indole building blocks could be directly used in the next reaction step, except for sulfoindole-AmineBoc, which was purified first.

### Example 11.1.2. Indole-Phth

A mixture of 2,3,3-trimethylindolenine (504 μL, 3.1 mmol) and N-(3-Bromopropyl)phthalimide (843 mg, 3.1 mmol) in 5 mL MeCN was stirred for 4 h at 100° C., followed by 72 h at 60° C. The resulting red precipitate was collected, dissolved in acetone and precipitated in Et2O. The suspension was filtrated and the residue was washed with Et2O yielding the crude product as an orange solid (1.2 g)

### Example 11.1.3. Indole-AmineBoc

A solution of 2,3,3-trimethylindolenine (3.7 ml, 22.8 mmol) and tert-butyl-(3-bromopropyl)carbamate (5.4 g, 22.8 mmol) in 25 ml dry MeCN was stirred for 72 h at 60° C. The mixture was concentrated under vacuum, re-dissolved in a small amount of MeOH and precipitated in Et2O while stirring. The precipitate was filtered off and washed with Et2O until the filtrate was colorless, yielding the product as a pink solid (3.5 g).

### Example 11.1.4. Sulfoindole-AmineBoc

A solution of 2,3,3-trimethyl-3H-indole-5-sulfonate potassium salt (1.1 g, 4 mmol) and 3-propylamine.HBr (0.87 g, 4 mmol) in 10 mL 1,2-dichlorobenzene was stirred for 30 min at 110° C., followed by 10 min at 150° C. The resulting purple precipitate was collected and dispersed in 15 mL MeOH. Di-tert-butyldicarbonate (1.7 g, 8 mmol) and DIPEA (1.4 mL, 8 mmol) were added and the reaction mixture was refluxed for 30 minutes. The mixture was concentrated in vacuo and purified by column chromatography (MeOH:CH2Cl2 1:3), yielding the product as a pink solid (81 mg).

### Example 11.2. Synthesis of the Cyanine-Dyes

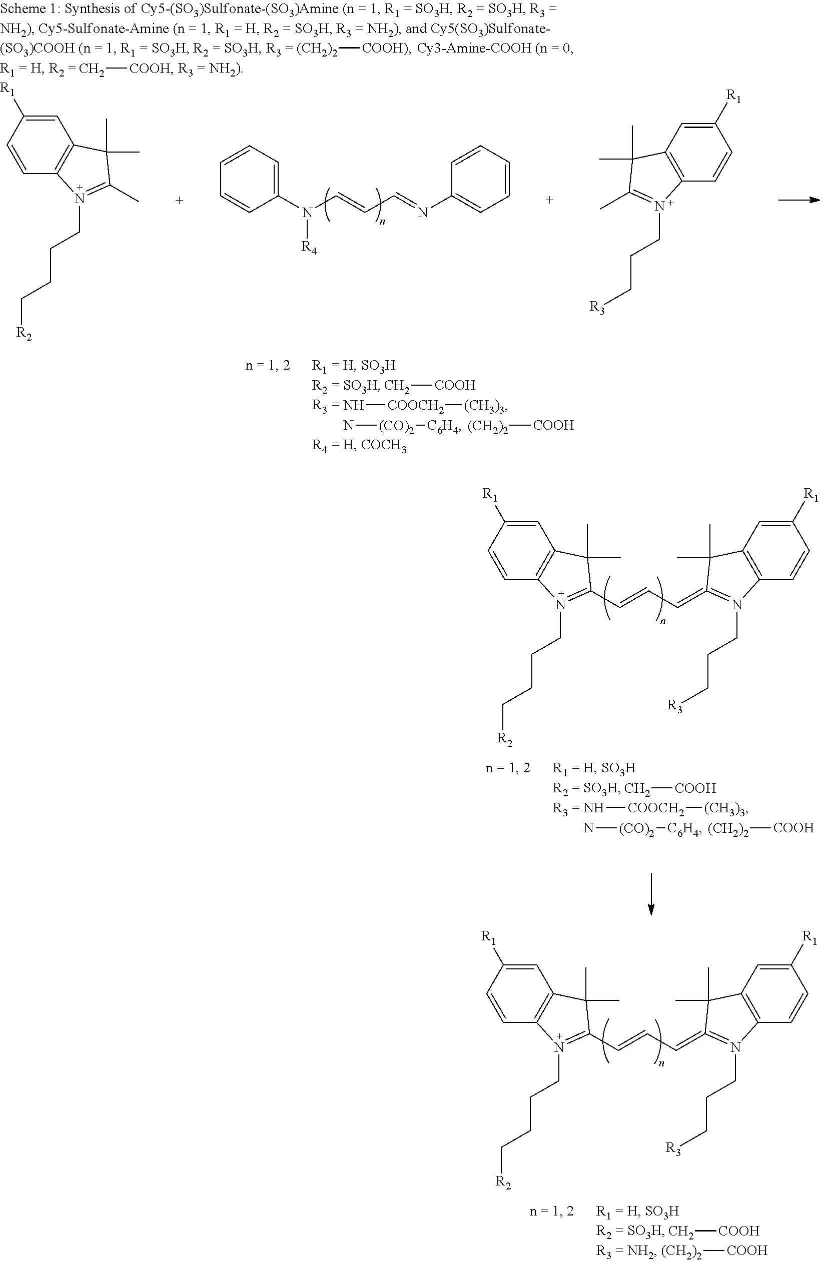

### Example 11.2.1. Cy5-(SO3)Sulfonate-(SO3)Amine

Sulfoindole-Sulfonate (41 mg, 0.1 mmol) and 3-anilinoacraldehyde anil hydrochloride (28 mg, 0.1 mmol) were dissolved in 4 mL HOAc:AC2O (1:1). After 30 minutes at 110° C., the compound was precipitated in 50 mL diethyl ether. The obtained solid was dissolved in a mixture of 8 mL Ac2O:Pyridine (1:1) and sulfoindole-AmineBoc (40 mg, 0.1 mmol) was added. After stirring at RT overnight, the product was concentrated under vacuo and purified by preparative HPLC. The product containing fraction was collected and lyophilized to give 5.0 mg (6.2 μmol) of Cy5-(SO3)Sulfonate-(SO3)AmineBoc. Subsequently, Cy5-(SO3)Sulfonate-(SO3)AmineBoc was deprotected by stirring in TFA:MeCN:CH2Cl2 2:2:1 overnight at RT. After evaporation of the solvents, Cy5-(SO3)Sulflonate-(SO3)Amine was obtained as a dark blue powder (4.4 mg, 6.2 μmol, 6% yield)

MS (MALDI-TOF): [C32H42N3O9S3]+ calcd 708.1, found 708.2. 1H NMR (300 MHz, D2O): 8.00 (m, 2H, CH), 7.83-7.74 (dd, 4H, Ar—H), 7.34 (d, 1H, Ar—H), 7.20 (d, 1H, Ar—H), 6.54 (t, 1H, CH), 6.34 (d, 1H, CH), 6.16 (d, 1H, CH), 4.09 (m, 4H, N—CH2), 3.10 (t, 2H, CH2-NH2), 2.96 (t, 2H, CH2-503), 2.15-1.91 (m, 6H, 3CH2), 1.61 (d, 12H, C—(CH3)2) ppm.

### Example 11.2.2. Cy5-Sulfonate-Amine

Indole-Sulfonate (100 mg, 0.34 mmol) and 3-anilinoacraldehyde anil hydrochloride (88 mm, 0.34 mmol) were dissolved in 10 mL HOAc:AC20 (1:1). After 60 min stirring at 110° C. the mixture was cooled down and a solution of Indole-AmineBoc (161 mg, 0.51 mmol) in 10 mL pyridine was added. After stirring for 2 h at 140° C., the blue solution was concentrated under vacuo and purified by column chromatography (MeOH:CH2Cl2 1:10 to 1:1 gradient) and preparative HPLC. Product containing fractions were collected and lyophilized to yield a black solid. Subsequently, the obtained solid was dissolved in 30 mL DCM:TFA (1:1) and a few drops of H2O were added. After 3 h stirring, the reaction mixture was concentrated under vacuo, redissolved in H2O and lyophilized to yield the product as a blue powder (11.6 mg, 21 μmol, 4.2% yield)

MS (MALDI-TOF): [C32H42N3O3S]+ calcd 548.7, found 548.5. 1H NMR (300 MHz, MeOD): 8.28 (m, 2H, CH), 7.53-7.23 (qt, 8H, Ar—H), 6.69 (t, 1H, CH), 6.47 (d, 1H, CH), 6.26 (d, 1H, CH), 4.18 (m, 4H, N—CH2), 3.12 (t, 1H, CH2-NH2), 2.93 (t, 2H, CH2-S03), 2.16-1.99 (m, 6H, 3CH2), 1.73 (s, 12H, C—(CH3)2) ppm.

### Example 11.2.3. Cy5-(SO3)Sulfonate-(SO3)COOH

Cy5-(SO3)Sulfonate-(SO3)COOH was synthesized according a previously reported method (Mujumdar, R. B.; Ernst, L. A.; Mujumdar, S. R.; Lewis, C. J.; Waggoner, A. S. Bioconjugate Chem 1993, 4, 105; Huveneers, S.; van den Bout, I.; Sonneveld, P.; Sancho, A.; Sonnenberg, A.; Danen, E. H. Cancer Res 2007, 67, 2693).

MS (MALDI-TOF): [C35H45N2O11S3]+ calcd 765.2, found 765.7. 1H NMR spectrum as previously described ((Mujumdar, R. B.; Ernst, L. A.; Mujumdar, S. R.; Lewis, C. J.; Waggoner, A. S. Bioconjugate Chem 1993, 4, 105; Huveneers, S.; van den Bout, I.; Sonneveld, P.; Sancho, A.; Sonnenberg, A.; Danen, E. H. Cancer Res 2007, 67, 2693)

### 11.2.4. Cy3-Amine-COOH

Synthesis of Cy3-Amine-COOH was adapted from previously described asymmetric cyanine synthesis (Song, J. W.; Cavnar, S. P.; Walker, A. C.; Luker, K. E.; Gupta, M.; Tung, Y. C.; Luker, G. D.; Takayama, S. PLoS One 2009, 4, e5756). N,N′-Diphenylformamidine (102 mg, 0.52 mmol) was dissolved in cold DCM (50 mL). DIPEA (181 μL, 1.04 mmol) and acetic anhydride (59 μL, 0.63 mmol) were added and the mixture was stirred for 2 h at room temperature. The mixture was concentrated under vacuum yielding a colorless oil. The oil was re-dissolved in 20 mL EtOH together with indole-COOH (127 mg, 0.46 mmol), indole-Phth (197 mg, 0.46 mmol) and pyridine (169 μL, 2.10 mmol). The solution was refluxed for 3 h and stirred overnight at 60° C. Acetic anhydride (60 μL) was added, and the reaction mixture turned pink. After refluxing for 4 h, the crude product was concentrated in vacuo and purified by column chromatography (eluens: MeOH). The product fractions were combined, concentrated and lyophilized to give a pink solid of impure compound Cy3-Phth-COOH. A portion of Cy3-Phth-COOH (100 mg, 0.16 mmol) was further purified by preparative HPLC. After lyophilization of the product fractions, Cy3-Phth-COOH was deprotected by adding 2 mL of CH3NH2 (33% in EtOH). The solution was stirred for 5 h, after which the reaction was concentrated in vacuo to give Cy3-Amine-COOH as a pink solid. Subsequently the compound was purified by preparative HPLC. Fraction containing product was collected and lyophilized and gave the pure product as a pink solid (4 mg, 7.99 μmol, 5% yield).

MS (MALDI-TOF): [C32H42N3O2]+ calcd 500.3, found 499.3. 1H NMR (500 MHz, DMSO-d6): 8.36 (t, 1H, CH), 7.66 (d, 2H, Ar—H), 7.51 (d, 2H, Ar—H), 7.46 (t, 2H, Ar—H), 7.32 (q, Ar—H), 6.55 (d, CH), 6.46 (d, 1H, CH), 4.21 (m, 2H, N—CH2), 4.12 (m, 2H, N—CH2), 2.94 (m, 2H, CH2-NH2), 2.22 (t, 2H, CH2-503), 2.03 (m, 2H, CH2), 1.74 (m, 2H, CH2), 1.71 (d, 12H, C—(CH3)2), 156 (m, 2H, CH2), 1.43 (m, 2H, CH2) ppm.

### Example 11.3. Synthesis of Ad-Functionalized Compounds

### Example 11.3.1. Ac-TZ14011-Ad

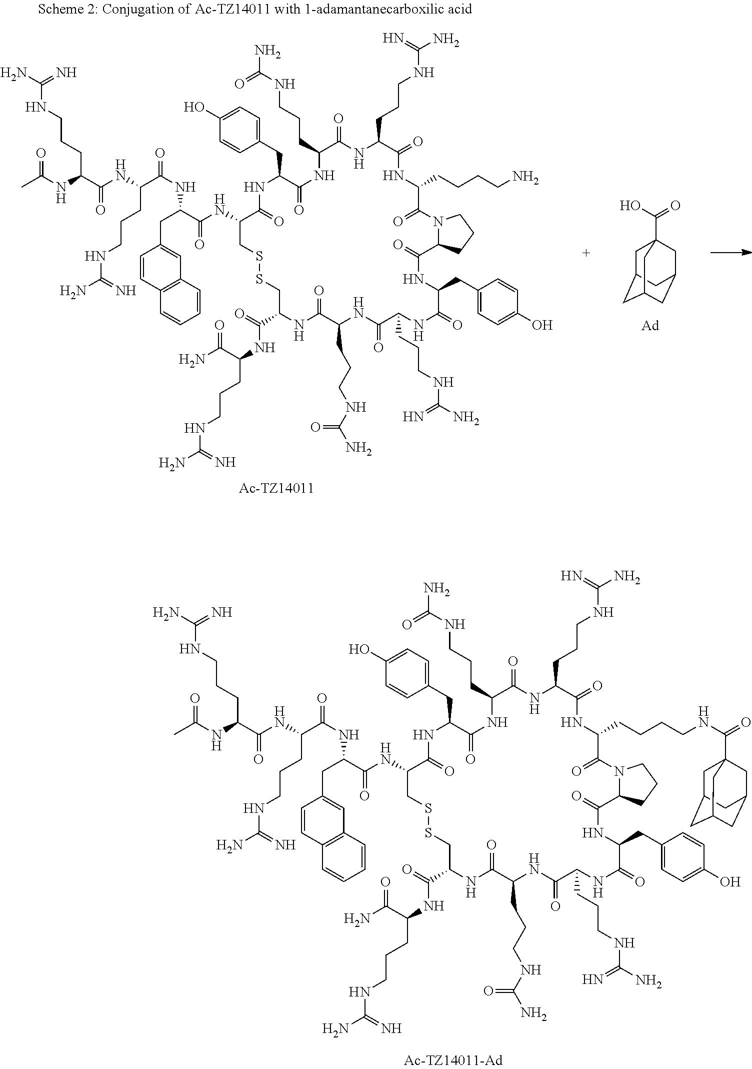

PyBOP (2.8 mg, 5.4 μmol), 1-adamantanecarboxylic acid (1.1 mg, 6 μmol) and DIPEA (5.1 μl, 30 μmol) were dissolved in 1 mL dry DMF and stirred for 5 minutes at RT. This was added to a solution of Ac-TZ14011 (9.8 mg, 3.5 μmol), synthesized as previously described (van den Berg et al., Transl Oncol, 2011, 4, 234), in 1 mL dry DMF. The reaction mixture was stirred for 48 h at RT. Subsequently, 2.5 mL of 0.1% TFA (H2O) was added to the reaction mixture to purify the reaction mixture directly by preparative HPLC. The product fraction was lyophilized to give the product as a white powder (8 mg, 2.7 μmol, 77% yield).

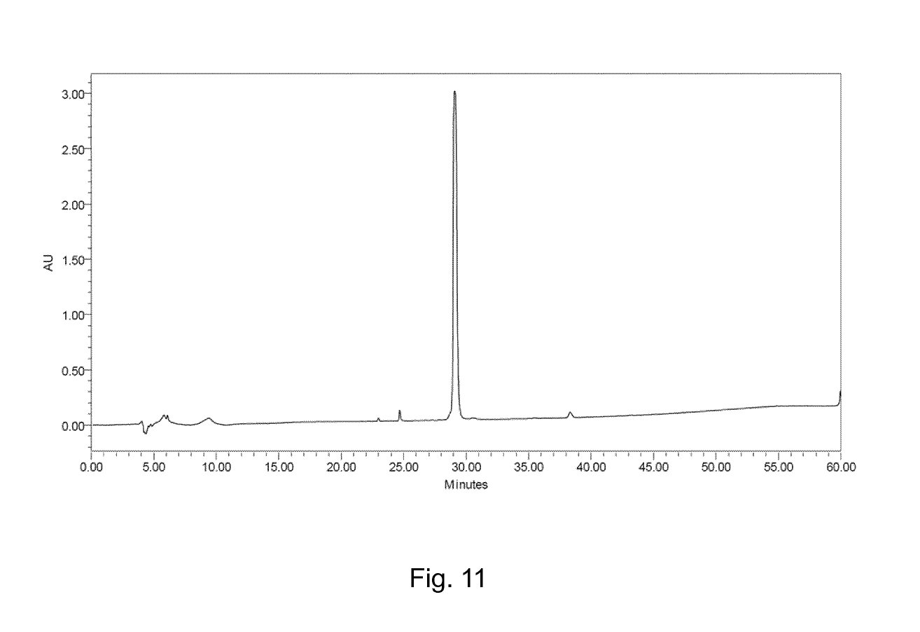

MS (MALDI-TOF): [C103H158N35O20S2]+ calcd 2270.7, found 2271.3. The analytical HPLC chromatogram is shown FIG. 11.

### Example 11.3.2. Cy5-Ad

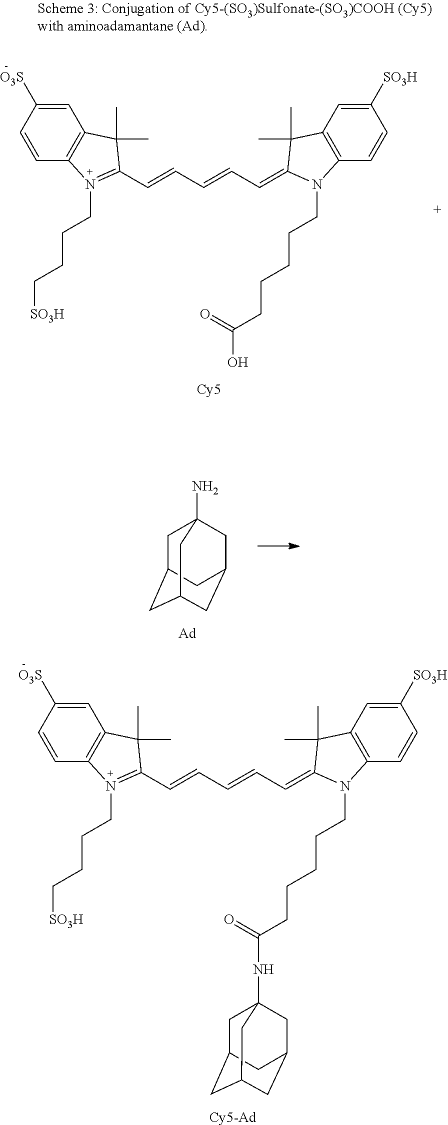

1—Aminoadamantane hydrochloride (7.5 mg, 40 μmol), Cy5-(SO3)Sulfonate-(SO3)COOH (8.2 mg, 10 μmol) and PyBOP (31 mg, 60 μmol) were dissolved in 2 mL dry DMF. DIPEA (50 μL, 300 μmol) was added and the reaction was stirred overnight, in the dark, at RT. Solvents were evaporated in vacuo and the product was purified by preparative HPLC. The product containing fraction was lyophilized to give the product as a blue powder (5.4 mg, 5.7 μmol, 57% yield).

MS (MALDI-TOF): [C45H60N3O10S3]+ calcd 898.3, found 898.9. 1H-NMR (300 MHz, DMSO-d6): 8.36 (t, 2H, CH), 7.80 (s, 2H, Ar—H), 7.61 (d, 2H, Ar—H), 7.31 (dd, 2H, Ar—H), 6.59 (t, 1H, CH), 6.34 (t, 2H, CH), 4.08 (m, 4H, N—CH2), 2.03-1.95 (m, 6H, 3CH2), 1.84 (s, 3H, 3CH), 1.80-1.69 (m, 2H, CH2), 1.68 (s, 12H, C—(CH3)2), 1.56 (s, 6H, 3CH2), 1.46 (m, 2H, CH2), 1.23 (s, 6H, 3CH2) ppm.

### Example 11.3.3. Cy5-Ad₂

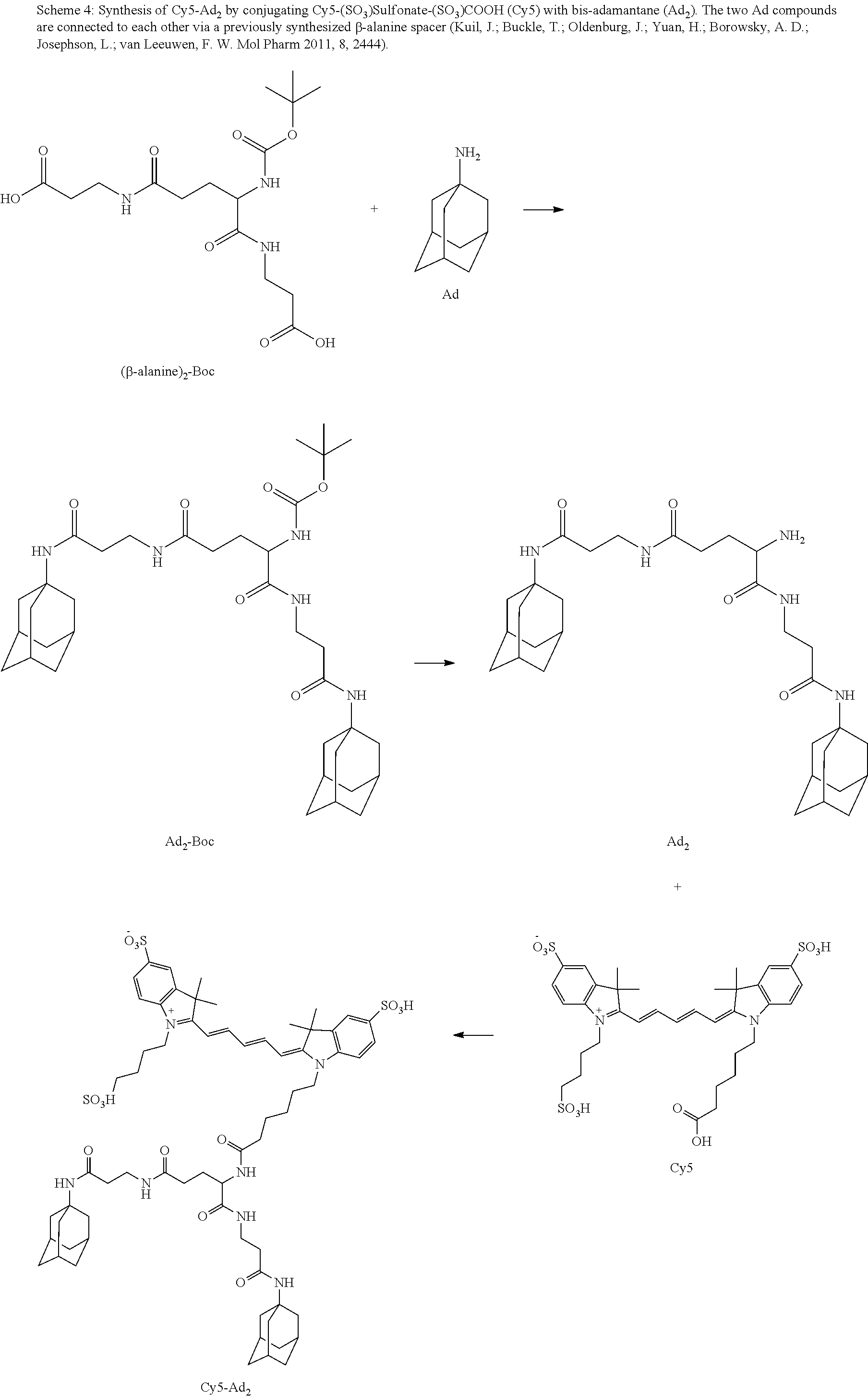

Cy5-Ad2 was synthesized in multiple steps using standard peptide coupling chemistry. Boc-Glu-(β-Ala)2 (101 mg, 0.26 mmol), synthesized as described before (Kuil, J.; Buckle, T.; Yuan, H.; van den Berg, N. S.; Oishi, S.; Fujii, N.; Josephson, L.; van Leeuwen, F. W. B. Bioconjugate Chemistry 2011, 22, 859), PyBOP (676 mg, 1.3 mmol) and DIPEA (530 μL, 3 mmol) were dissolved in 5 mL dry DMF. After 5 min, adamantan-1-amine hydrochloride (244 mg, 1.3 mmol) was added and the mixture was stirred for 3 h at room temperature. The product was purified by column chromatography (CH2Cl2:MeOH 9:1) and the product containing fractions were collected and concentrated under vacuo. The obtained compound was dissolved in 4 mL TFA:DCM (1:4) and stirred overnight at RT. After evaporation of the solvents in vacuo, the obtained H-Glu-(β-Ala-Ad)2 was dissolved in H2O:MeCN and lyophilized to give the product as a white solid (111 mg, 0.17 mmol). Subsequently, H-Glu-(β-Ala-Ad)2, (20 mg, 30 μmol) was dissolved in 2 mL dry DMF and Cy5-(SO3)Sulfonate-(SO3)COOH (25 mg, 30 μmol), PyBOP (16 mg, 30 μmol) and DIPEA (17 μL, 100 μmol) were added. After stirring overnight at RT, 2 mL of 0.1% TFA in H2O was added to purify the product directly by preparative HPLC. The product containing fractions were collected and lyophilized to give Cy5-Ad2 as a blue solid (9.0 mg, 6.9 μmol, 2.6%).

MS (MALDI-TOF): [C66H92N7O14S3]+ calcd 1303.7, found 1303.9. 1H-NMR (300 MHz, DMSO-d6): 8.35 (t, 2H), 7.80 (s, 2H), 7.60 (d, 2H), 7.31 (dd, 2H), 6.58 (t, 1H), 6.34 (m, 2H), 4.07 (m, 4H), 2.0-1.0 (m, 10H), 1.84 (s, 6H), 1.68 (s, 12H). 1H NMR (300 MHz, DMSO-D6): 8.35 (t, 2H, CH), 7.80 (s, 4H, Ar—H), 7.60 (d, 2H, Ar—H), 7.33 (d, 2H, Ar—H), 6.58 (t, 1H, CH), 6.42 (d, 1H, CH), 6.31 (d, 1H, CH), 4.07 (m, 4H, N—CH2), 3.36-3.15 (m, 5H, CH and 2CH2, semi covered under solvent peak), 2.29-2.02 (m, 6H, 3CH2), 2.01-1.19 (m, 10H, 5CH2), 1.88 (s, 6H, 6CH), 1.80-1.69 (m, 4H, 2CH2) 1.68 (s, 12H, C—(CH3)2), 1.57 (s, 12H, 6CH2), 1.23 (s, 12H, 6CH2) ppm.

### Example 11.4. Synthesis of Cy5-CD

Cy5-(503)Sulfonate-(503)COOH (4.2 mg, 5 μmol), 6-monodeoxy-6-monoamino-6-cyclodextrin (5.9 mg, 5 μmol), and PyBOP (5.2 mg, 10 μmol) were dissolved in 1 mL dry DMSO. DIPEA (3.5 μL, 20 μmol) was added and the mixture was stirred overnight at RT. The products were precipitated in CH2Cl2 and purified by preparative HPLC. The product fraction was lyophilized to give a blue powder (5.2 mg, 2.7 μmol, 53%).

MS (MALDI-TOF): [C77H113N3O44S3]+ calcd 1880.8, found 1882.0. 1H NMR (300 MHz, D2O): 8.03 (m, 2H, CH), 7.79-7.74 (m, 4H, Ar—H), 7.29 (t, 2H, Ar—H), 6.6-6.2 (m, 3H, CH), 4.97 (m, 7H, O—CH—O—CH of β-CD), 4.05 (m, 4H, N—CH2), 3.90-3.49 (m, 42H, all other β-CD protons), 2.91 (t, 2H, CH2-COOH), 2.17 (t, 2H, CH2-503), 2.0-1.0 (m, 10H, 5CH2), 1.65 (s, 12H, C—(CH3)2) ppm.

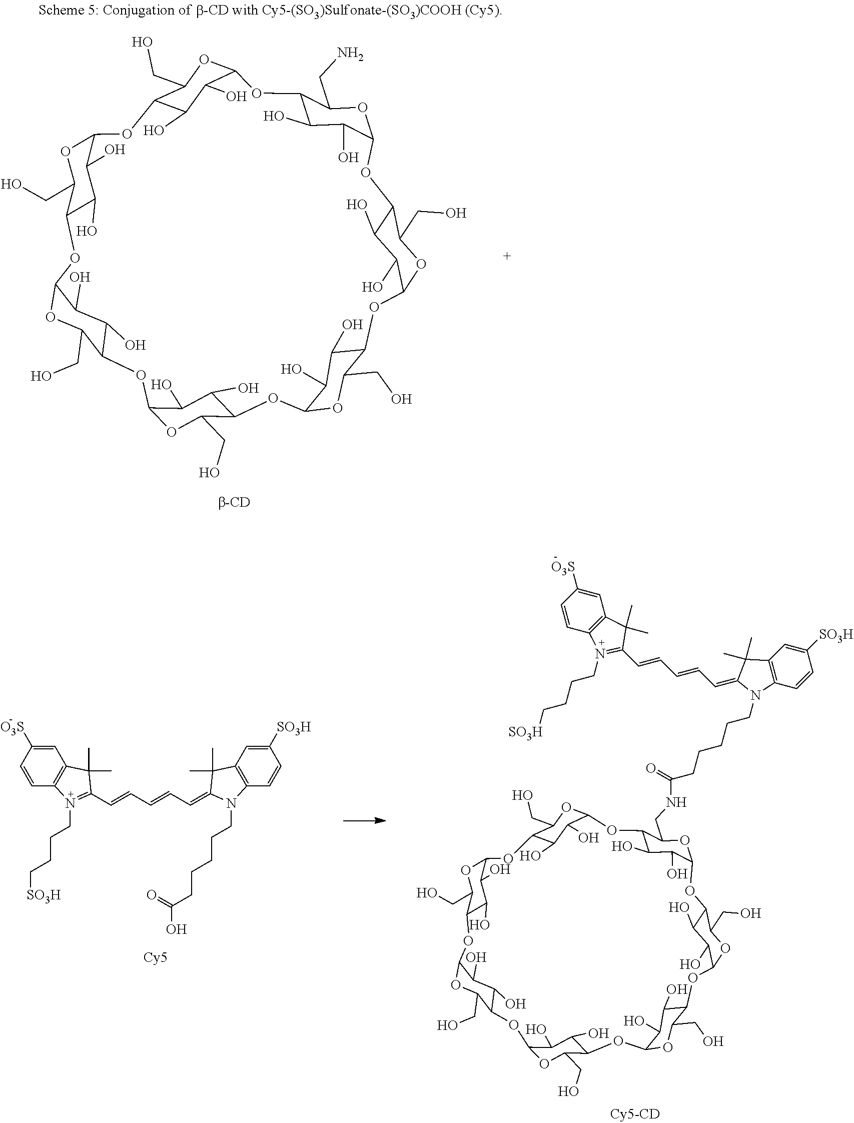

### Example 11.5. Synthesis of the Polymers

### General Description

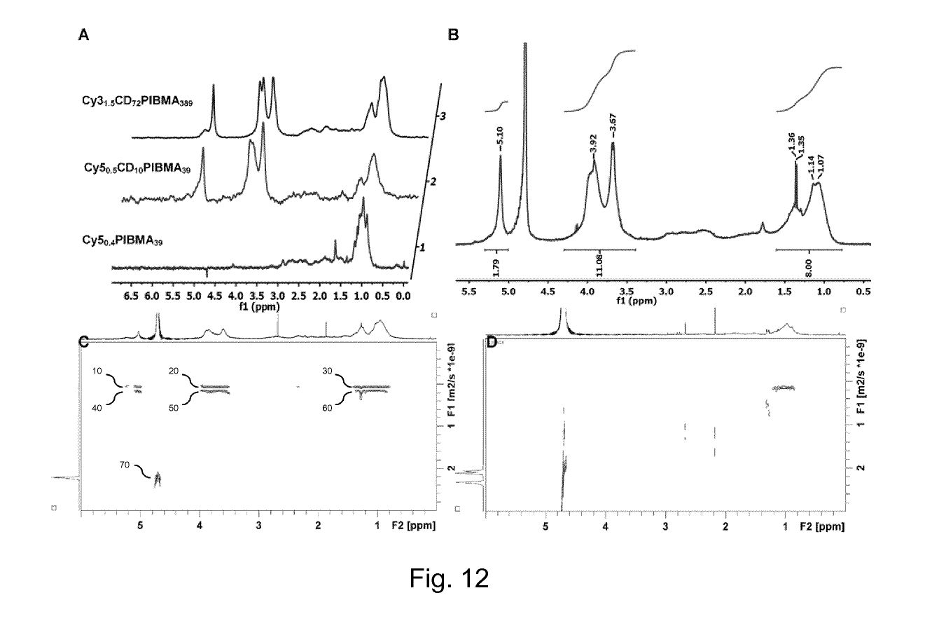

Poly(isobutylene-alt-maleic anhydride) (PIBMA₃₉, Mw 6,000) or PIBMA₃₈₉ (Mw 60,000) were dissolved in dry DMSO together with DIPEA and the appropriate Cy5- or Cy3-dye. The reaction was left to stir for at least 7 h. Then 6-monodeoxy-6-monoamine-β-cyclodextrin (β-CD) was added and the mixture was stirred at 80° C. for another 12 h. After cooling to RT, the polymer was dialyzed against H2O for 1 day, then against 100 mM phosphate buffer pH 9.0 for another day, and finally against H2O for 5 days. The dialysis medium was refreshed every day. The remaining solution was then lyophilized to obtain the product. The number of CD groups per polymer, was estimated via 1H NMR analysis and the number of dyes per polymer was estimated via UV/Vis absorbance (see example 11.5.4., Table 1 and FIG. 12). Prior to use, a solution of 1 mg/mL in H2O was prepared of each polymer.

### Example 11.5.1. Synthesis of Cy5_{0.4} PIBMA₃₉

PIBMA₃₉ (9.1 mg, 1.5 μmol) and Cy5-Sulfonate-Amine (1 mg, 1.8 μmol) were dissolved in 0.6 mL dry DMSO and DIPEA (13 μL, 74 μmol) was added. The reaction was stirred at RT overnight. No 6-monodeoxy-6-monoamino-β-cyclodextrin was added and the reaction mixture was directly dialyzed according above described procedure. After lyophilisation, the product was obtained as a blue powder (2 mg, 0.3 μmol). Average number of Cy5 dye per polymer according UV/vis absorbance: 0.4 Estimated molecular weight: 7.8 kDa (Table 1)

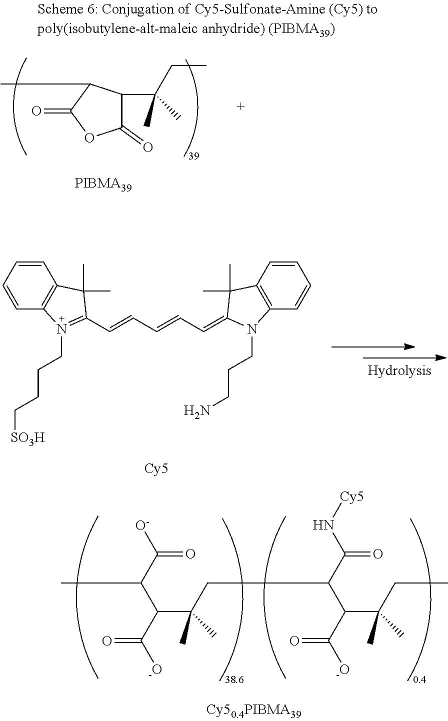

### Example 11.5.2. Synthesis of Cy5_{0.5}CD₁₀PIBMA₃₉

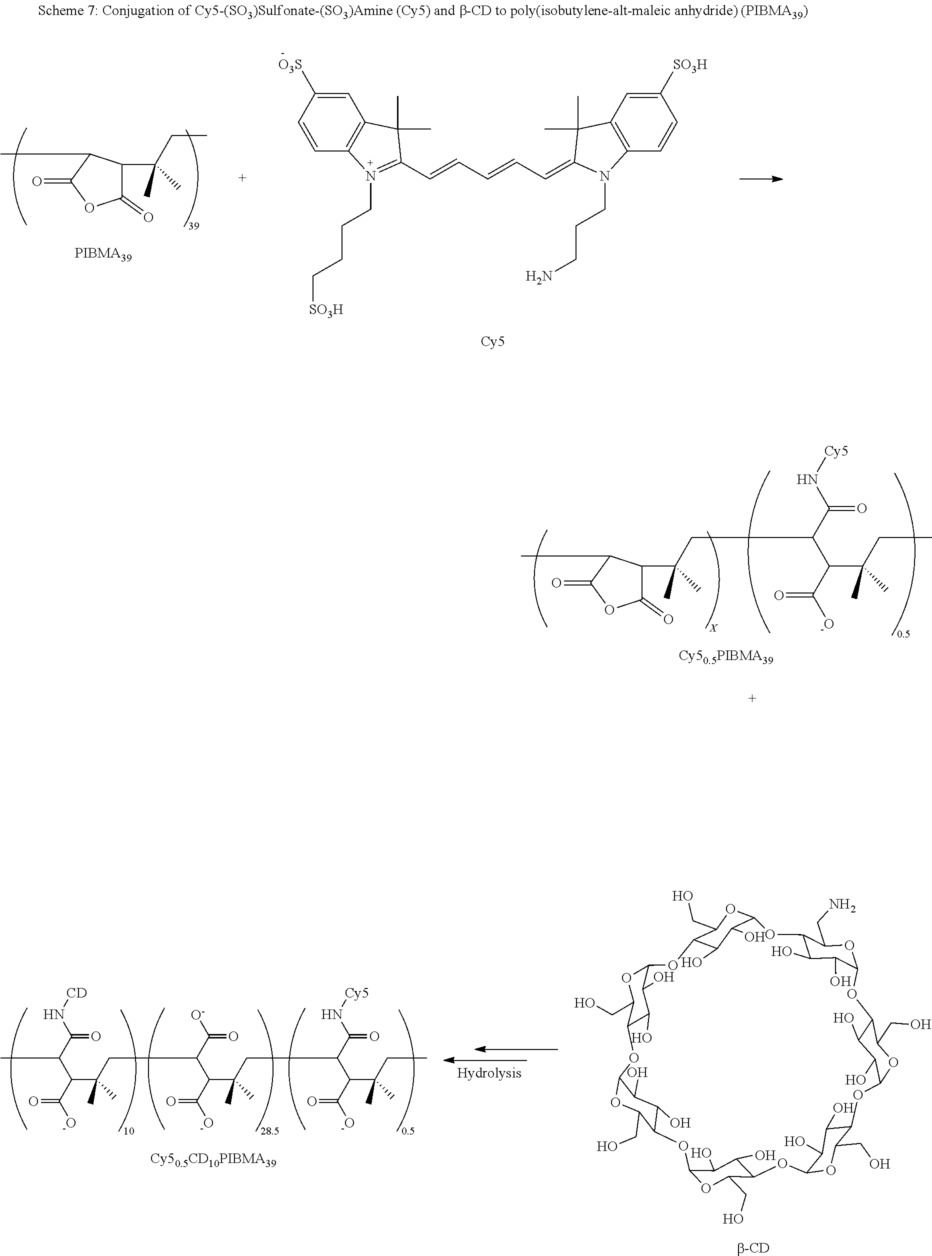

PIBMA₃₉ (30 mg, 5 μmol) and Cy5-(SO3)Sulfonate-(SO3)Amine (5.0 mg, 5.6 μmol) were dissolved in 3 mL dry DMSO and DIPEA (50 μL, 250 μmol) was added. The reaction was stirred at 80° C. for 7 h, then 6-monodeoxy-6-monoamino-β-cyclodextrin (95 mg, 80 μmol) was added and the solution was stirred for another 72 h at 80° C. After dialysis and lyophilisation, the product was obtained as a blue powder (87 mg, 5 μmol).

Average number of CD groups per polymer according 1H NMR: 10
Average number of Cy5 dye per polymer according UV/vis absorbance: 0.5
Estimated molecular weight: 18.8 kDa (Table 1)

### Example 11.5.3. Synthesis of Cy3_{1.5}CD₇₂PIBMA₃₈₉

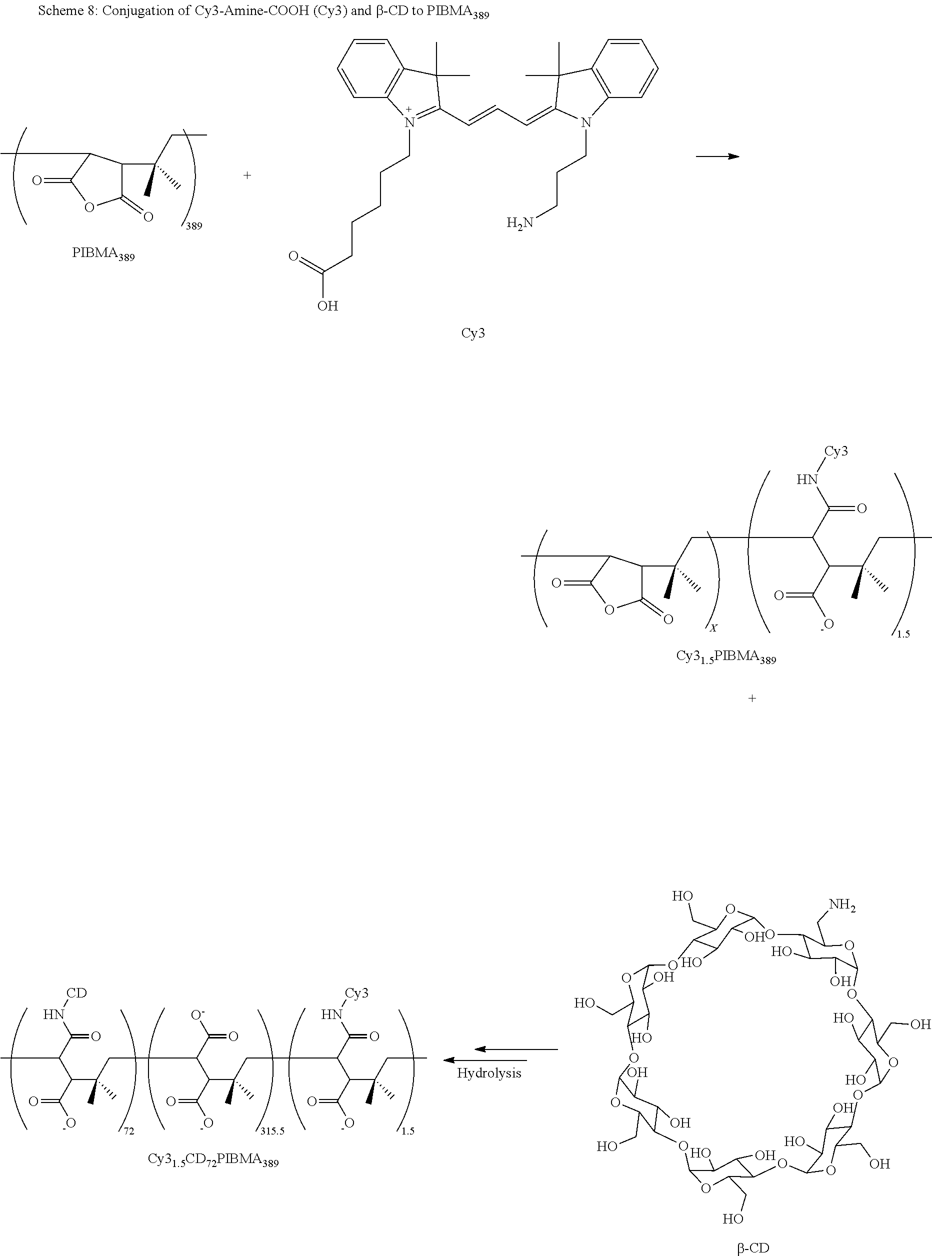

PIBMA₃₈₉ (10 mg, 0.17 μmol) and DIPEA (15 μL, 85 μmol) were dissolved in 1.7 mL dry DMSO and a solution of Cy3-Amine-COOH in dry DMSO (0.25 mM, 800 μL, 0.2 μmol) was added. The reaction was stirred overnight at RT, then, 6-monodeoxy-6-monoamino-β-cyclodextrin (31.6 mg, 27 μmol) was added and the solution was stirred for another night at 80° C. After dialysis and lyophilisation the product was obtained as a bright pink powder (22.3 mg, 0.16 μmol).

Average number of CD groups per polymer according 1H NMR: 72
Average number of Cy3 dye per polymer according UV/vis absorbance: 1.5
Estimated molecular weight: 155 kDa (Table 1)

### Example 11.5.4. Analysis of the CD-Polymers

The grafting efficiency of β-CD and the fluorophores was determined by a combination of 1H-NMR and UV/Vis absorption measurements. The grafting of the β-CD was determined by 1H-NMR, by integrating the polymer peaks at 1.38-1.00 ppm (—(CH3)2-C-CH2-) and the β-CD peaks at 5.1 ppm (—O—CH—O—CH—) and 4.00-3.50 ppm (all other β-CD protons). The integral of the peaks corresponding to the polymer was then set at 8 (FIG. 12B). The obtained integral for the β-CD peaks at 5.1 ppm and 4.00-3.50 ppm were divided by 7 and 42 respectively (in theory, if one β-CD per subunit would have been present, their integrals would have been 7 and 42). The resulting ratio is the approximated percentage of β-CD per subunit. Both ratios obtained for the peak at 5.0 and 4.0-3.5 ppm were averaged and then multiplied by the number of subunits in the polymer.

To determine the number of fluorophores per polymer, first the fluorophore concentration of the sample was calculated by measuring the absorbance at 650 nm (Cy5) or at 550 nm (Cy3) and applying the Beer-Lambert law (equation 1). Then the concentration of the fluorophore was correlated with the calculated concentration of the polymer, based on its estimated molecular weight.

A=l·ε·C (eq.1)

Where: A=absorbance, l=path length in cm, ε=absorption coefficient, C=Molar concentration

The molecular weight (MW) of the polymer was first estimated by adding together: the starting MW of the polymer (6,000 or 60,000 g/mol), the MW of β-CD (1133 g/mol) times the numbers of β-CD per polymer (0, 10, or 72), and the MW of H2O (18 g/mol) times the number of carboxylates per polymer (78.0, 67.5, or 705.5). Then the number of fluorophores per polymer was calculated and the resulting number of fluorophore per polymer (0.4, 0.5, or 1.5) times the MW of the fluorophore was added to obtain the final MW of the polymers

The hydrodynamic radii of the polymers were determined using dynamic light scattering (DLS) and diffusion-ordered NMR spectroscopy (DOSY). Based on the diffusion constants, the hydrodynamic radii could be calculated using the Stokes-Einstein equation (Equation 2). Unfortunately, the presence of Cy5 on Cy5_{0.4}PIBMA₃₉ and Cy5_{0.5}CD₁₀PIBMA₃₉ disturbed the DLS measurements for these two compounds and the dynamic radius was only based on DOSY.

$$D = \frac{{k}_{B} T}{6 πη r} \qquad ( eq . 2 )$$

Where: D=diffusion constant, KB=boltzmann's constant, T=absolute temperature, η=dynamic viscosity of the medium and r=radius of the particles/compound.

TABLE 1
Determined parameters of the synthesized polymers

| | | | | | | MW | Ø | Ø |
|---|---|---|---|---|---|---|---|---|
| | MW start | Repeating | CD/ | Carboxylates/ | Dye/ | final | (DLS) | (DOSY) |
| | (kDa) | units | polymer | polymer | polymer | (kDa) | (nm) | (nm) |
| Cy5_{0.4}PIBMA₃₉ | 6 | 39 | 0 | 78 | 0.4 | 7.8 | ^{a} | 2.7 |
| | | | | | Cy5 | | | |
| Cy5_{0.5}CD₁₀PIBMA₃₉ | 6 | 39 | 10 | 67.5 | 0.5 | 18.8 | ^{a} | 2.8 |
| | | | | | Cy5 | | | |
| Cy3_{1.5}CD₇₂PIBMA₃₈₉ | 60 | 389 | 72 | 704.5 | 1.5 | 155.0 | 18.4 | 11.7 |
| | | | | | Cy3 | | | |

^{a} Values could not be determined due to Cy5 influence.

### Example 11.6. Cell Culture

Human MDA-MB-231 cells, transfected with human CXCR4 conjugated GFP (MDA-GFP-CXCR4+), were kindly provided by Dr. Gary Luker (Center for Molecular Imaging, University of Michigan, USA) (Song et al., PLoS One 2009, 4, e5756). Native MDA-MB-231 cells with basal CXCR4 expression were used as control (van den Berg et al., Transl Oncol 2011, 4, 234). Cells were maintained in Dulbecco's minimum essential medium (DMEM) enriched with 10% fetal bovine serum and 5 mL Penicillin/Streptomycin (1,0000 units/mL Penicillin; 1,0000 μg/mL Streptomycin) (all Life Technologies Inc., Breda, The Netherlands). Cell lines were cultured and maintained under standard conditions (37° C. and 5% CO2).

### Example 11.7. Confocal Microscopy

One day prior the experiment, cells were trypsinized, seeded onto culture dishes with glass insert (035 mm glass bottom dishes No. 15, poly-d-lysine coated, γ-Irradiated, MatTek corporation) and incubated overnight in 2 mL DMEM. After incubation of the compounds (see below for the conditions of each experiment), live cell images were taken on a Leica SP5 or SP8 WLL confocal microscope under 63× magnification. The intrinsic GFP signal in the MDA-GFP-CXCR4+ cells was measured with excitation at 488 nm and emission was collected at 500-525 nm. Cy3 fluorescence was measured with excitation at 514 nm, emission was collected at 550-570 nm. Cy5 fluorescence was measured with excitation at 633 nm, emission was collected at 650-700 nm. Any Hoechst 33342 fluorescence was measured using 405 nm excitation and emission was collected at 420-470 nm. Images and signal quantifications were obtained using Leica Application Suite software, by applying the polygon function and calculate the average gray value/m2 for each cell. For quantifications; background signal (amount of grey value/m2 when no compounds are added) was subtracted from the fluorescence signal obtained for the samples.

### Example 11.8. Flow Cytometry

MDA-GFP-CXCR4+ cells were trypsinized (using 0.5% trypsin/EDTA, BD Biosciences) and counted. Hereafter cells were divided into aliquots (300,000 cells per tube), centrifuged for three minutes (3000×g, 4° C.), and the supernatant was decanted. After incubation of the compounds (see below the detailed conditions per experiment) flow cytometry measurements were performed on a BD FacsCanto™ II. Live cells were gated using forward scatter and side scatter, and 10,000 viable cells were analyzed for each sample. Cy5 fluorescence was measured on the APC channel and Cy5.5 fluorescence (for Ac-TZ14011-MSAP) was measured on the APC-Cy7 channel. For quantification; background signal (amount of fluorescence when no functionalizations were added) was subtracted from the fluorescence signal obtained for all samples.

### Example 12. Functionalization of Cells: Using Ac-TZ14011-Ad in Combination with CD-Polymers

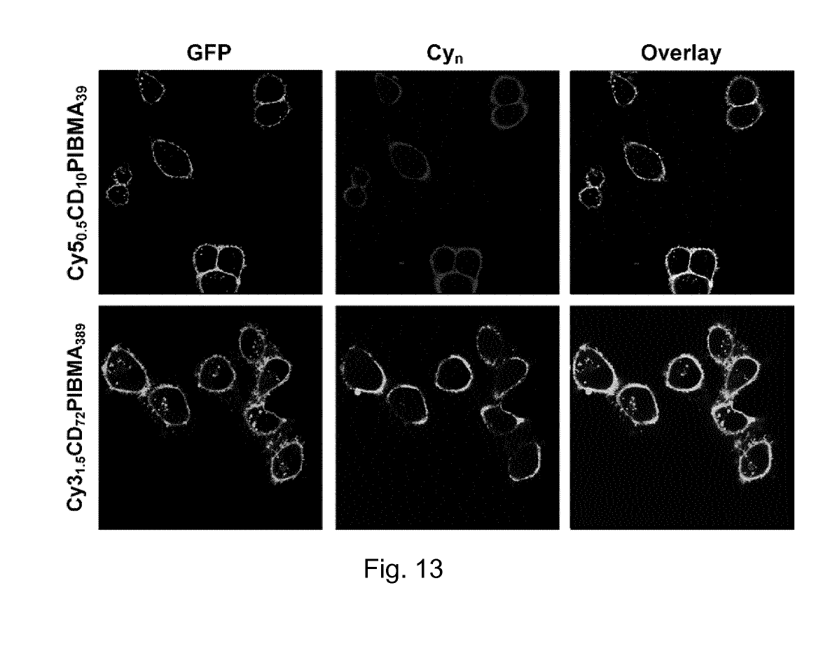

To proof (supramolecular) cell-surface modification becomes possible via specific functionalization of the membrane-receptors, CXCR4 overexpressing MDA-GFP-CXCR4+ cells were functionalized in two steps; first with Ac-TZ14011-Ad (1 h; 0° C.), to allow for CXCR4 receptor targeting (step 1) and secondly with either Cy5_{0.5}CD₁₀PIBMA₃₉ or Cy3_{1.5}CD₇₂PIBMA₃₈₉ (1 h; 0° C.) to allow further surface functionalization (Step 2): MDA-GFP-CXCR4+, were seeded onto culture dishes (80,000 per dish) as described in the example 11.7. Cells were brought to 0° C., followed by incubation with Ac-TZ14011-Ad (11 μM) in 1 mL DMEM for 1 h at 0° C. Subsequently, either Cy5_{0.5}CD₁₀PIBMA₃₉ or Cy3_{1.5}CD₇₂PIBMA₃₈₉ was added (10 μM final β-CD concentration). After 1 h of incubation at 0° C., cells were washed twice with PBS. Cell analysis using confocal microscopy indicated that cell functionalization was accomplished using both polymer types: As can be seen from the confocal images shown in FIG. 13 the cells displaying the CXCR4 receptor on the surface as indicated by emission of green light on both images in the left-most column (GFP) are also emitting either red or blue light indicating functionalization by Cy5_{0.5}CD₁₀PIBMA₃₉ (upper row) or Cy5_{0.5}CD₁₀PIBMA₃₉ (lower row), respectively.

### Example 13. Functionalization in a Mixed Cell-Culture Set-Up

To further study the CXCR4-receptor specificity of the functionalization process, the experiment described in example 12 was repeated with a mixed cell culture of viable MDA-GFP-CXCR4+(with overexpressed CXCR4 receptor and with GFP-tag) and as a control MDA-MB-231 cells (with basal CXCR4 expression and without GFP-tag). First, a mixture of 40,000 cells of each strain of MDA-GFP-CXCR4+ and MDA-MB-231 cells were seeded. The next day, the cells were brought to 0° C. and subsequently they were functionalized with either Cy5_{0.5}CD₁₀PIBMA₃₉ or Cy3_{1.5}CD₇₂PIBMA₃₈₉ (see functionalization of the cells). During confocal analysis discrimination between the fluorescence of the outer membrane of the two cell lines was based on the GFP signal, which was only present in the MDA-GFP-CXCR4+ strain (not shown). Confocal microscopy (not shown) and intensity analysis revealed that the signal intensity of Cy5_{0.5}CD₁₀PIBMA₃₉ and Cy3_{1.5}CD₇₂PIBMA₃₈₉ were respectively 5 and 8 times higher on the MDA-GFP-CXCR4+ cells, compared to the signal intensity found on the cells with basal CXCR4 expression (MDA-MB-231), indicating receptor specificity.

### Example 14. Ac-TZ14011-Ad Induced Cell Functionalization Analyzed by Confocal Microscopy

The influence of Ac-TZ14011-Ad on the degree of Cy5_{0.5}CD₁₀PIBMA₃₉ functionalization was examined by varying the conditions of the first incubation step as follows; 1) by omitting the use of a CXCR4-binding peptide, 2) by using non-Ad functionalized Ac-TZ14011 or 3) via the standard procedure by using Ac-TZ14011-Ad. Differences in Cy5_{0.5}CD₁₀PIBMA₃₉ functionalization between the three set-ups were analyzed using both semi-quantitative (confocal microscopy) and quantitative (flow cytometry) methods.

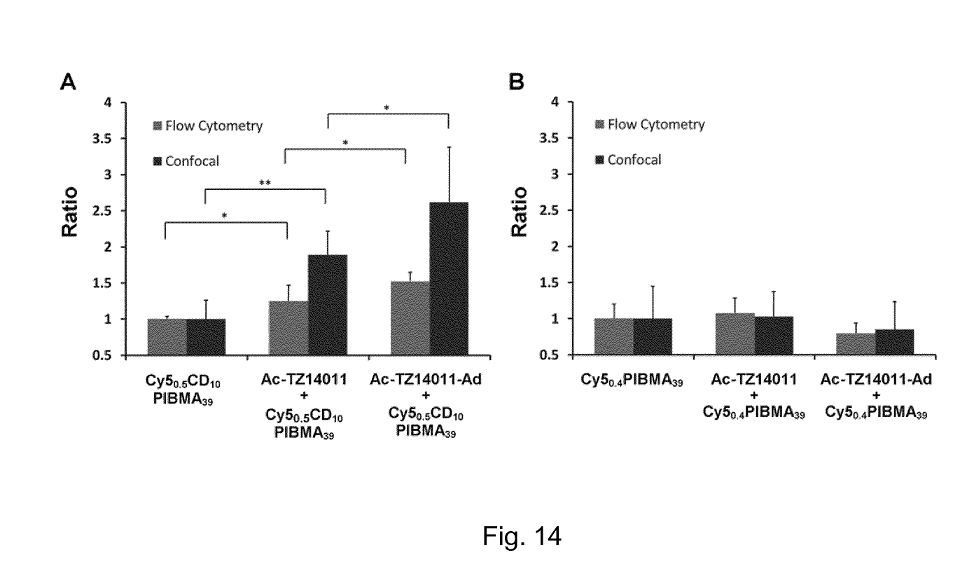

For Confocal microscopy MDA-GFP-CXCR4+ cells (80.000 per well) were incubated with either Ac-TZ14011 (10 μM), Ac-TZ14011-Ad (10 μM), or none, for 1 h at 0° C. in 1 mL DMEM. Subsequently, either Cy5_{0.4}PIBMA₃₉ or Cy5_{0.5}CD₁₀PIBMA₃₉ was added (10 μM β-CD; 1 μM polymer final concentration) and another hour at 0° C. of incubation followed. Thereafter, the cells were washed two times with PBS and confocal images were acquired. All experiments were performed in 6-fold. The Cy5 signal present on the cell in each sample was quantified to analyze the differences between the amount of binding of the polymers to the cells when either, no peptide, Ac-TZ14011, or Ac-TZ14011-Ad was present. For normalization (after background substraction), all results were divided by the average fluorescence value obtained when just the polymer was added. The significance of the obtained differences was determined by student T-test (two tailed, unpaired) (FIG. 14).

For flow cytometry MDA-GFP-CXCR4+ cells were incubated with 50 μL PBS containing either Ac-TZ14011 (10 μM), Ac-TZ14011-Ad (10 μM), or none for 1 h at 0° C. Subsequently, 50 μL of either Cy5_{0.4}PIBMA₃₉ or Cy5_{0.5}CD₁₀PIBMA₃₉ in PBS was added (10 μM β-CD; 1 μM polymer final concentration) and another hour at 0° C. of incubation followed. The cells were washed two times with PBS (centrifuged 3 min, 3000×g, 4° C.), resuspended in 300 μL PBS and the intensity of Cy5 fluorescence related to the cells was measured by flow cytometry as described earlier. All experiments were performed in 8-fold. For normalization of the data, all results were divided by the average fluorescence value obtained when only the polymer is added without first functionalization with either Ac-TZ14011 or Ac-TZ14011-Ad. The significance of the obtained differences was determined by student T-test (two tailed, unpaired) (FIG. 14).

Although the two techniques give a difference in absolute values, both depict the same trend and significance therein (FIG. 14A). When the influence of β-CD was eliminated by using Cy5_{0.4}PIBMA₃₉ as polymer, no difference in binding was observed in relation to the presence of the Ac-TZ14011-Ad peptide (FIG. 14B). this finding indicate that β-CD-Ad interactions have an undisputable role in the functionalization process. FIG. 14 further suggests that the Ac-TZ14011 peptide also interacts with β-CD. Besides hydrophobic organic guests, β-CD is known to host hydrophobic amino acids like tryptophan and tyrosine (Aachmann et al., Protein Eng 2003, 16, 905; Uekama et al., Chem Rev 1998, 98, 2045). When bound to the receptor, Ac-TZ14011 has one solvent exposed tyrosine (Tyr10) available for possible β-CD interactions (Scheme 1) (Kuil et al., Chem Soc Rev 2012, 41, 5239). It can however be concluded that the combined presence of the Ac-TZ14011 peptide and the Ad-functionality yields a significant increase in Cy5_{0.5}CD₁₀PIBMA₃₉ binding. Together these findings underline that using host-guest interactions, in this example in the form of Ad- and β-CD play a crucial role in the functionalization process.

### Example 15. Comparison of Multivalent (Cy5_{0.5}CD₁₀PIBMA₃₉) and Monovalent (Cy5-CD) Functionalization

Individual CXCR4 receptors have a diameter of approximately 4 to 5 nm, based on the crystal structure of CXCR4 obtained from the protein data bank (PDB code 3OE0) (Kuil et al., Chem Soc Rev 2012, 41, 5239). Although the distance between CXCR4 receptors on the membrane is unknown, it is reported that CXCR4 can cluster in groups (Singer et al., J Virol 2001, 75, 3779; Nicolson, Biochim Biophys Acta 2014, 1838, 1451). When assuming a spherical structure, Cy5_{0.5}CD₁₀PIBMA₃₉ has a hydrodynamic diameter of 2.8 nm in water but, when unfolded, the polymer length is approximately 24 nm (based on the estimated bond lengths of one subunit, times the number of subunits in the polymer). Hypothetically, this should allow simultaneous interactions with multiple (clustered) Ac-TZ14011-Ad functionalized CXCR4 receptors. The longer Cy3_{1.5}CD₇₂PIBMA₃₈₉ polymer (hydrodynamic diameter ˜11.7 nm; unfolded >200 nm) should certainly allow such multivalent interactions. To test this theory, the functionalization was performed with monovalent Cy5-CD instead of a CDₙPIBMAₘ polymer.

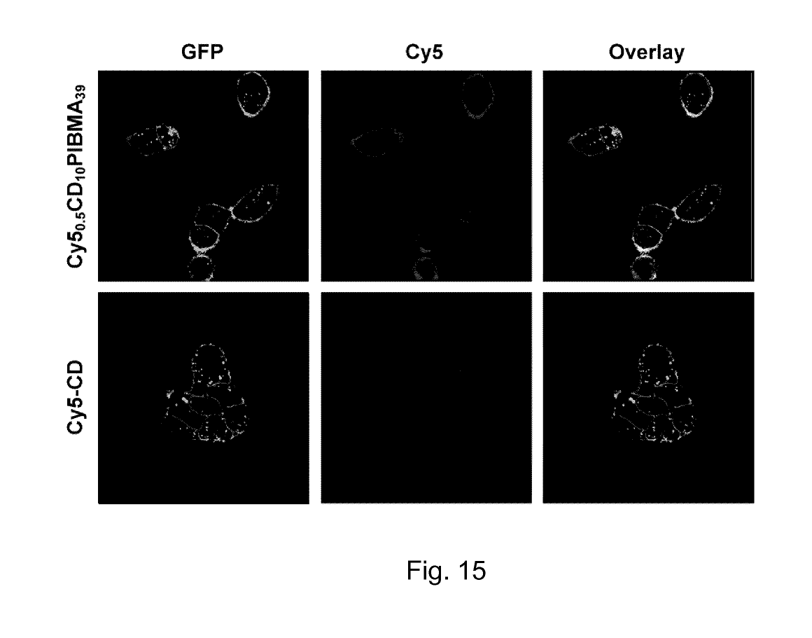

To study the differences in binding of multivalent- and monovalent β-CD compounds to Ac-TZ14011-Ad functionalized cells, adhering MDA-GFP-CXCR4+ cells were functionalized with either Cy5_{0.5}CD₁₀PIBMA₃₉, or a mixture of Cy5-CD and 6-monodeoxy-6-monoamino-β-cyclodextrin (according to the procedure described in example 12), in such a way that for both conditions the β-CD concentration was 10 μM and the Cy5 concentration was 0.5 μM. After washing twice with PBS, confocal images were taken (FIG. 15). As can be seen from FIG. 15, cells functionalized with the Cy5-CD monomer showed only a very low degree of cell functionalization, indicating a each β-CD-host molecule may interact with at least two different Ad-guest molecules and thereby indirectly with at least two or more Ac-TZ14011-Ad functionalized CXCR4 receptors.

### Example 16. Competition Between Cy5_{0.5}CD₁₀PIBMA₃₉ and Cy3_{1.5}CD₇₂PIBMA₃₈₉ Followed Over Time

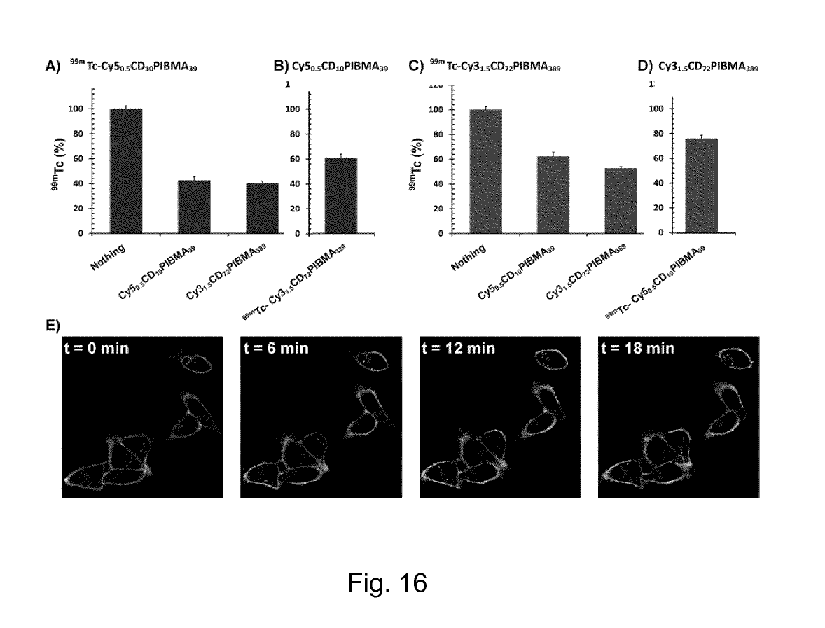

To monitor the strength and reversibility of the PIBMA surface modifications, competition experiments between Cy5_{0.5}CD₁₀PIBMA₃₉ and Cy3_{1.5}CD₇₂PIBMA₃₈₉ were performed and studied by confocal microscopy and by flow cytometry. The results of these experiments are shown in FIG. 16.

### Example 16.1. Competition Between Cy5_{0.5}CD₁₀PIBMA₃₉ and Cy3_{1.5}CD₇₂PIBMA₃₈₉ Visualized by Confocal Microscopy

The competition between Cy5_{0.5}CD₁₀PIBMA₃₉ and Cy3_{1.5}CD₇₂PIBMA₃₈₉ was visualized by confocal microscopy. Adherent MDA-GFP-CXCR4+ cells were functionalized with Cy5_{0.5}CD₁₀PIBMA₃₉ (3.6 μM final β-CD concentration), washed with PBS (2×1 mL) and imaged with confocal microscopy at RT. Subsequently, Cy3_{1.5}CD₇₂PIBMA₃₈₉ (4.3 μM final β-CD concentration) was added and the change in fluorescence, as a measure for competition was followed for 20 minutes, while taking images each minute. (FIG. 16E).

The results indicated that over the 20 minutes, all Cy5_{0.5}CD₁₀PIBMA₃₉ had been out-competed by Cy3_{1.5}CD₇₂PIBMA₃₈₉. This is visualized in FIG. 16E, which shows that the color of the light emitted from the cell surface changes from red and green at time=0 minutes (corresponding to GFP and Cy5) to blue and green at time=18 minutes (corresponding to GFP and Cy3). At time=6 minutes some blue light is already emitted from the surface but red light is also still emitted but at time=12 minutes there is very little red light emitted, indicating that most. These results indicate that at the teste competitor concentration, most of the Cy5_{0.5}CD₁₀PIBMA₃₉ was out-competed already after 12 minutes.

### Example 16.2. Competition Between Cy5_{0.5}CD₁₀PIBMA₃₉ and Cy3_{1.5}CD₇₂PIBMA₃₈₉ Quantified by Radioactivity

To monitor the strength and reversibility of the PIBMA surface modifications, competition experiments were performed between radiolabelled and non-radiolabelled CDₙPIBMAₘ polymers. After functionalizing MDA-GFP-CXCR4+ cells with either 99mTc-Cy5_{0.5}CD₁₀PIBMA₃₉, 99mTc-Cy3_{1.5}CD₇₂PIBMA₃₈₉, Cy5_{0.5}CD₁₀PIBMA₃₉ or Cy3_{1.5}CD₇₂PIBMA₃₈₉ the polymers remained attached to the cell membrane for at least 1 hour in PBS, before the competitor was added.

Radiolabeling of Cy5_{0.5}CD₁₀PIBMA₃₉ and Cy3_{1.5}CD₇₂PIBMA₃₈₉ with technetium-99m was performed as follows: Cy5_{0.5}CD₁₀PIBMA₃₉ (75 μL, 43.8 nmol CD) or Cy3_{1.5}CD₇₂PIBMA₃₈₉ (75 μL, 38 nmol CD), was mixed with SnCl2.2H2O (4 μL of 1 mg/mL saline solution, 17.7 nmol, Technescan PYP Kit, Mallinckrodt Medical B.V., Petten, The Netherlands) and freshly eluted 99mTc-Na-pertechnetate (200 μL, Technekow, Mallinckrodt Medical B.V.) and gently stirred for 1 h at RT. Thereafter, the reaction mixture was purified from free 99mTc by size exclusion chromatography using sterile PBS as mobile phase on Sephadex™ G-25 desalting columns (PD-10, GE Healthcare Europe GmbH, Freiburg, Germany). Fractions containing radiolabeled Cy5_{0.5}CD₁₀PIBMA₃₉ (99mTc-Cy5_{0.5}CD₁₀PIBMA₃₉) or Cy3_{1.5}CD₇₂PIBMA₃₈₉ (99mTc-Cy3_{1.5}CD₇₂PIBMA₃₈₉) were collected and directly applied in the assembly studies at a final concentration of 13 μM).

MDA-GFP-CXCR4+ cells were harvested, counted and diluted to 80,000 cells/mL using DMEM. Of this solution, 200 μL (16,000 cells) fractions were transferred to polystyrene tube (FACS) and the cells were cooled on ice for 15 minutes. Subsequently, Ac-Tz14011-Ad (100 μL, 11 μM) was added and another incubation of 15 minutes followed. Thereafter, the mixture was centrifuged (5 min, 1250×g, 4° C.) and the supernatant was aspirated. The cells were resuspended in 200 μL DMEM and either 99mTc-Cy5_{0.5}CD₁₀PIBMA₃₉, 99mTc-Cy3_{1.5}CD₇₂PIBMA₃₈₉, Cy50.5CD10PIBMA39, or Cy3_{1.5}CD₇₂PIBMA₃₈₉ (100 μL, 13 μM) was added, and incubation followed for 15 min at 0° C. Thereafter, the mixture was centrifuged as described before. After aspiration of the supernatant and resuspension of the cells in 200 μL DMEM, either Cy5_{0.5}CD₁₀PIBMA₃₉, Cy3_{1.5}CD₇₂PIBMA₃₈₉, 99mTc-Cy5_{0.5}CD₁₀PIBMA₃₉, or 99mTc-Cy3_{1.5}CD₇₂PIBMA₃₈₉ (100 μL, 13 μM) was added and the samples were shortly vortexed. To challenge the strength of the binding of the fluorescent β-CD polymers to MDA-GFP-CXCR4+ cells, incubation of 1 h at 0° C. followed. After centrifugation of the cells and aspiration of the supernatant the tubes were counted for radioactivity in a dose-calibrator or gamma counter to assess the amount of cellular-bound 99mTc-Cyₙ-CDₓ-PIBMA_{y} activity. Data was expressed as the mean % (±SD, n=6) of the total amount of 99mTc-Cyₙ-CDₓ-PIBMA_{y} activity added to the cells (FIG. 16A-D).

Using either Cy5_{0.5}CD₁₀PIBMA₃₉ or Cy3_{1.5}CD₇₂PIBMA₃₈₉ as competitors for the cell-bound radiolabeled versions of these polymers resulted in a decrease in signal (FIGS. 16A and C). Binding of the competitors was confirmed when the experiment was performed with non-radioactive Cy5_{0.5}CD₁₀PIBMA₃₉ or Cy3_{1.5}CD₇₂PIBMA₃₈₉ functionalized MDA-GFP-CXCR4+ cells and using 99mTc-labeled competitors, indicating an exchange between the two polymers (FIGS. 16B and D). The difference in length between the two polymers did not seem to influence the affinity at equal b-CD quantities, since both polymers could compete with each other. The polymer exchange was visually confirmed using confocal microscopy experiments performed over time (0-18 min), at the same b-CD concentrations (FIG. 16E and example 16.1.). When Cy3_{1.5}CD₇₂PIBMA₃₈₉ is added to Cy5_{0.5}CD₁₀PIBMA₃₉ functionalized MDA-GFP-CXCR4+ cells, the Cy5 signal is slowly replaced by Cy3. Combined, these data suggest that the polymer functionalization is stable without the presence of a competitor, and becomes reversible when a competitor is present.

### Example 17. Cy5-Ad and Cy5-Ad₂ Functionalization of Polymer Modified Cell Surfaces

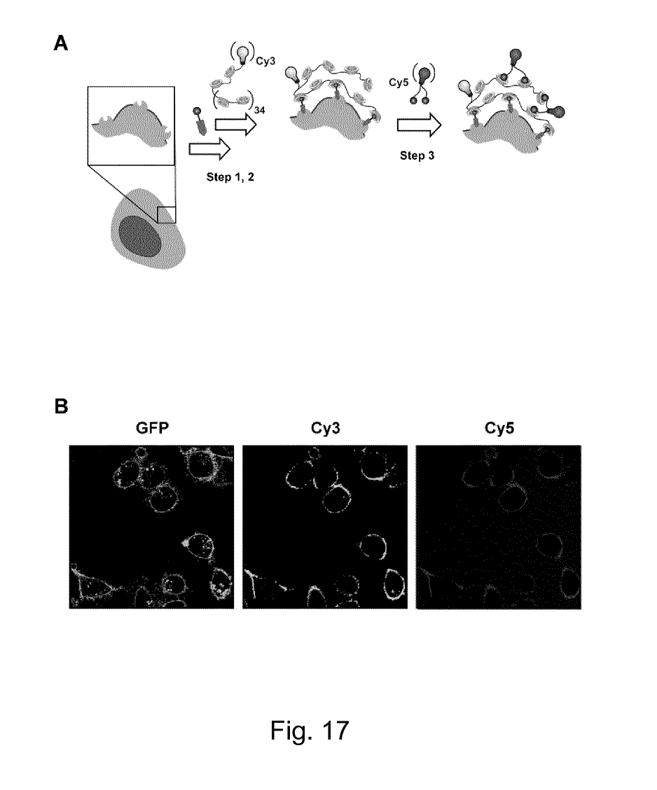

A third functionalization on the β-CD polymer functionalized cells was introduced, by first functionalizing adherent MDA-GFP-CXCR4+ cells with Cy3_{1.5}CD₇₂PIBMA₃₈₉. Subsequently, the cells were washed once with DMEM, followed by incubation with y Cy5-Adn (n=1 or 2, 5 μM) in 1 mL DMEM for 1 h at 0° C. followed. As a control experiment, the cells were incubated with Cy5-Ad2 (5 μM final concentration) while the polymer was omitted in the first incubation step. The resulting functionalization of the cells was determined using confocal microscopy (FIG. 17).

The monovalent Cy5-Ad, showed very little staining of cells that were pre-functionalized with Cy3_{1.5}CD₇₂PIBMA₃₈₉ (data not shown). In contrast, the bivalent Cy5-Ad2 showed clear staining under the same conditions, providing co-localization of the CXCR4 receptor (GFP), Cy3_{1.5}CD₇₂PIBMA₃₈₉ (Cy3), and Cy5-Ad2 (Cy5) (FIG. 17B). Control experiments where Cy5-Ad2 was added in the absence of Cy3_{1.5}CD₇₂PIBMA₃₈₉, did not yield non-specific staining (data not shown). When looking at the binding constant of mono- and bivalent Ad with multivalent β-CD hosts in general (Gade et al., Chem Commun (Camb) 2015, 51, 6346; Huskens et al., JACS 2004, 126, 6784; Mulder et al., JACS 2004, 126, 6627; Granadero et al., Int J Mol Sci 2010, 11, 173), a difference of at least a factor 200 is found. The binding constant of bis-adamantane (e.g. Cy5-Ad2) with multivalent β-CD hosts lies between 1 107-1 1010 M-1 (depending on the host and its environment) (Huskens et al., JACS 2004, 126, 6784; Mulder et al., JACS 2004, 126, 6627), while the Cy5-Ad interaction with Cy3_{1.5}CD₇₂PIBMA₃₈₉ can be seen as a monovalent interaction of which the binding constant lies around 5 104 M-1 (Gade et al., Chem Commun (Camb) 2015, 51, 6346; Granadero et al., Int J Mol Sci 2010, 11, 173). Again multivalency of in this case at least two guest functional groups seems to be a key component for facilitating stable interactions under in vitro conditions. The ability to utilize β-CD functionalized cell surfaces to introduce a third-generation of functionalization opens up a scale of functionalization types to tailor a wide range of applications. For example, diagnostic labels for cell-tracking53 could be introduced via this route. Alternatively, the introduction of therapeutic agents or a combination of both is possible. Hereby cells are converted into functional scaffolds that can be applied for delivery applications.

### Example 18. Cell-Cell Interactions

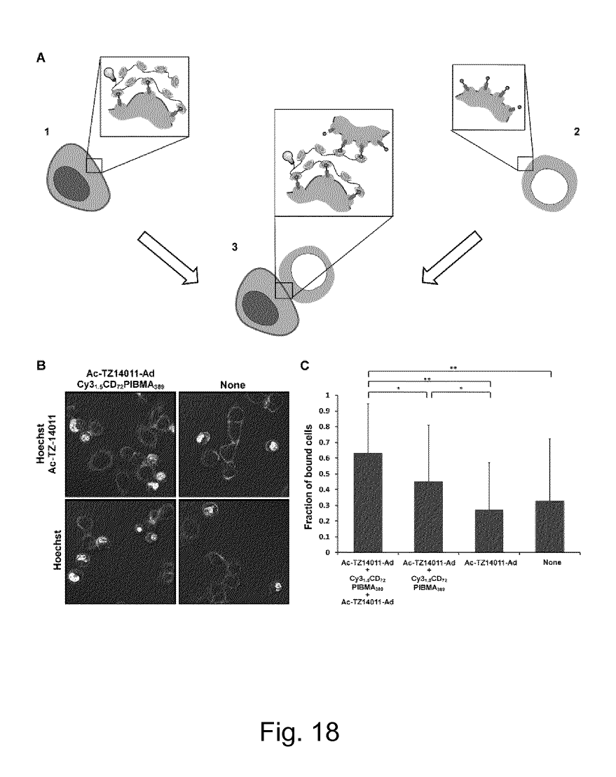

Given the fact that the CDₙPIBMAₘ polymers interact with Ac-TZ14011-Ad functionalization on the cell surface and that the secondary polymer surface functionalization enables a third-generation of surface modifications, we reasoned it would be of interest to use such technology to drive the interactions between MDA-GFP-CXCR4+ cells that are either functionalized with CDₙPIBMAₘ polymers or Ac-TZ14011-Ad (FIG. 18A).

To study cell-cell interactions, variable combinations of functionalized MDA-GFP-CXCR4+ cells were combined. MDA-GFP-CXCR4+ cells (300,000 per tube) in suspension were incubated with Hoechst 33342 (1 μg/mL) for 30 minutes in 1 mL DMEM at 0° C. Subsequently, they were washed once with PBS (centrifuged 3 min, 3000×g, 4° C.), cooled on ice and either incubated with Ac-TZ14011-Ad (11 μM) or none, in DMEM (500 μL) for 1 h at 0° C. After washing twice with PBS (centrifuged 3 min, 3000×g, 4° C.), cells were resuspended in 300 μL PBS and added to a separate batch of adherent MDA-GFP-CXCR4+ target cells (80,000 cells per dish). The latter were either functionalized with Cy3_{1.5}CD₇₂PIBMA₃₈₉ or none, and subsequently washed with PBS. The variable cell mixtures, see Table 2, were allowed to incubate in 1 mL PBS for 15 to 30 min at RT. Prior to imaging, the excess of unbound cells in suspension were gently washed away with PBS (2×1 mL, RT).

TABLE 2
Tested cell mixtures of adhered and suspended
MDA-MB-231 cells and their functionalization.

| | | In suspension MDA-MB-231 + |
|---|---|---|
| Cell mixture | Adherent MDA-MB-231 | Hoechst labelled |
| 1 | Ac-TZ14011-Ad + | Ac-TZ14011-Ad |
| | Cy3_{1.5}CD₇₂PIBMA₃₈₉ | |
| 2 | Ac-TZ14011-Ad + | None |
| | Cy3_{1.5}CD₇₂PIBMA₃₈₉ | |
| 3 | None | Ac-TZ14011-Ad |
| 4 | None | None |

The samples were examined under confocal microscopy in a culture dish, of each sample approximately 10 images were acquired at different randomly chosen locations. All experiments were performed in 5-fold. For each image obtained, the ratio between Hoechst stained cells that had an interaction with a target cell and the total number of Hoechst-stained cells in the image, was calculated. Obtained ratio's for each cell combination (Table 2) were averaged and a statistical significance of differences between each cell combination determined using student T-test (two tailed, unpaired)(FIG. 18). This analysis of the obtained images revealed that on average 61% of the Hoechst stained suspended cells within the field of view showed interactions with non-Hoechst stained adherent cells. Control experiments where the adherent cells were not functionalized using Cy3_{1.5}CD₇₂PIBMA₃₈₉ and/or in which the cells in suspension were not functionalized with Ac-TZ14011-Ad resulted in significantly lower percentages of cell-cell interactions (FIG. 18C). These results indicate that the introduced cell-surface modifications and underlying supramolecular chemistry also has the potential to drive cell-cell interactions.

### Example 19. Functionalization of Human Stem Cells

To demonstrate that the above technology is applicable to other cell types and in particular to stem cells, human fetal heart stem cells were functionalized as follows: Human fetal heart stem cells, with CXCR4 expression, (17 weeks after gestation) were grown in M199−/− on gelatin-coated glass-bottom dishes (100,000 cells per dish). These cells were functionalized with Ac-TZ14011-Ad and Cy3_{1.5}CD₇₂PIBMA₃₈₉ according to the procedure described in Example 12.

Subsequently, they were carefully washed with colorless DMEM (2×1 mL) and analyzed by confocal microscopy, which showed cells emitting blue light from their cell surfaces indicating that they had been functionalized with Cy3_{1.5}CD₇₂PIBMA₃₈₉ (Figure S10 data not shown).

### Example 20. Determination of the Receptor Affinity of Ac-TZ14011-Ad

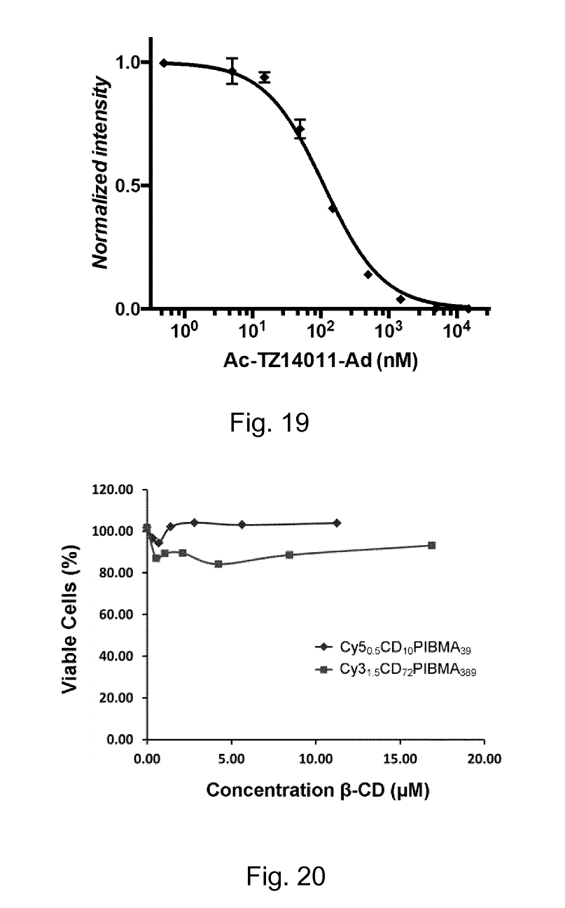

The receptor affinity of Ac-TZ14011-Ad was determined by flow cytometry-based competition experiments on viable CXCR4 expressing cells (MDA-GFP-CXCR4+). The affinity (K_{D}) of Ac-TZ14011-Ad was calculated from flow cytometry measurements, using an earlier described procedure (Kuil et al., Bioconjugate Chemistry 2011, 22, 859). In short: Different concentrations of Ac-TZ14011-Ad, ranging between 0.5-15000 nM in 120 μL PBS, were added to MDA-GFP-CXCR4+ cells in the presence of Ac-TZ14011-MSAP (250 nM), a compound with well defined receptor affinity (Kuil et al., Bioconjugate Chemistry 2011, 22, 859). After one hour of incubation on ice, the cells were washed two times with PBS (centrifuged 3 min, 3000×g, 4° C.), and resuspended in 300 μL PBS. The fluorescence of the reference compound was measured as described in the flow cytometry section. All experiments were performed in duplicate (n=2). The mean fluorescence was normalized and fitted with equations in the GraphPad Prism 6 software (FIG. 19). The K_{D} values were calculated using the “Binding-Competitive, One site-Fit Ki” nonlinear regression equation (Eq. 3 and 4), where the used K_{D} value of Ac-TZ14011-MSAP (187 nM) has previously been reported (Kuil et al., Bioconjugate Chemistry 2011, 22, 859.

$$\begin{matrix}log {IC}_{50} = log ( {10}^{log {K}_{D}} * ( 1 + \frac{[ MSAP ]}{[ {K}_{D , MSAP} ]} ) ) & ( eq . 3 ) \\ y = \frac{1}{1 + {10}^{x - log {IC}_{50}}} & ( eq . 4 )\end{matrix}$$

IC₅₀=concentration of the competitor that results in 50% binding, K_{D}=dissociation constant of the competitor in nM, [MSAP]=concentration of Ac-TZ14011-MSAP (250 nM), K_{D,MSAP}=dissociation constant of Ac-TZ14011-MSAP (187 nM), y=normalized fluorescence,

x=concentration of peptide-Ad in nM.

This experiment revealed a K_{D} of 56 nM for Ac-TZ14011-Ad (FIG. 19), using the fluorescent Ac-TZ14011-MSAP (K_{D}=187 nM) as a reference. Unmodified Ac-TZ14011 has an affinity of 8.6 nM (Kuil et al., Mol Pharm 2011, 8, 2444), indicating that the Ad functionality has relatively small adverse effect on the receptor affinity (Kuil et al., Chem Soc Rev 2012, 41, 5239).

### Example 21. Cell Viability Assay

To determine the effect of polymer functionalization on the cell viability, after cell functionalization a MTT test was performed according to a described procedure (Mosmann, T. J Immunol Methods 1983, 65, 55; Gerlier and Thomasset, N. J Immunol Methods 1986, 94, 57). MDA-GFP-CXCR4+ cells (16,000 cells per tube) in 200 μL DMEM were cooled on ice for 15 minutes. Ac-Tz14011-Ad (100 μL, 10 μM) was added and incubation of 15 minutes followed. Thereafter, the mixture was centrifuged (5 min, 1250×g, 4° C.) and the supernatant was aspirated. The cells were resuspended in 200 μL DMEM and variable concentrations of either Cy5_{0.5}CD₁₀PIBMA₃₉ or Cy3₁₅CD₁₀PIBMA₃₈₉ (100 μL, 0-16 μM final β-CD concentration) were added. After 15 min incubation on ice, the cells were washed two times with PBS (centrifuged 5 min, 1250×g, 4° C.), resuspended in 400 μL DMEM (16,000 cells per tube) and transferred to a 96-wells plate (Cellstar®, Greiner Bio-One, Alphen a/d Rijn, The Netherlands) with 8,000 cells per well in a volume of 200 μL DMEM. After 24 h incubation at 37° C., 20 μL (0.1 mg) of a MTT (3-(4,5-dimethylthiazol-2-yl)-2,5-diphenyltetrazolium bromide) solution in PBS (5 mg/mL) was added to each well, followed by gently shaking the plate and incubation for 4 h at 37° C. Thereafter, cells were optically checked under a light microscope to determine the uptake and colouring of MTT. The medium was carefully removed and 100 μL of DMSO was added to each well to extract the insoluble formazan product. After 10-15 minutes at 37° C., the uptake of MTT in each well was determined by measuring the absorbance at 545 nm. Measurements were normalized to those of untreated cells which were kept on ice during the entire procedure of polymer functionalization (FIG. 20).

### Example 22. Functionalization with a Multivalent Guest Structure as a Second Layer of Functionalization

### Synthesis of Ad₁₂₀-Polymer (5)

ISOBAM-04 (50 mg, 0.83 μmol), 100 equivalents of adamantan-1-yl-2-aminoacetamide (3) (30 mg, 83 μmol), and DIPEA (83 μL, 0.4 mmol) were added to 5 ml dry DMSO and the reaction was stirred overnight at 80° C. After cooling to RT, and dialysis in a manner identical to that described above, the solution was lyophilized to give 49.3 mg of a white solid.

The grafting efficiency of the adamantane groups on the polymer backbone was determined by ¹H-NMR measurements. Comparing the integral of the glycine spacer peak (3.7 ppm) with those of the polymer backbone (3.0-0.8 ppm) revealed that the polymer was functionalized with on average 120 Ad groups, resulting in an estimated molecular weight of 90 kDa and a estimated reaction yield of 66%.

Synthesis of Ad₁₅₀-Cy5_{0.5}-Polymer (6)

ISOBAM-04 (50 mg, 0.83 μmol), 1.2 equivalents of Cy5-NH₂ (1.0 μmol) and DiPEA (83 μL, 0.4 mmol) were added to 5 ml dry DMSO and the reaction was stirred 80° C. overnight. Adamantan-1-yl-2-aminoacetamide (3) (30 mg, 83 μmol) was added, and the reaction was stirred again at 80° C. overnight. After cooling to RT and dialysis in a manner identical to that described above, the solution was lyophilized to give a blue solid (46 mg).

The grafting efficiency of the adamantane groups was determined similar to 5, revealing that 6 was functionalized with on average 150 Ad groups. The grafting efficiency of the fluorophore groups was determined by UV/Vis spectroscopy. A fraction of the polymer was weighted and dissolved in H2O to a concentration of 1 mg/ml. UV/Vis measurements of this solution revealed that on average 0.5 Cy5 groups (ε=250,000 M⁻¹ cm⁻¹) were grafted to each polymer. Combining the NMR and UV/Vis data resulted in a final estimated molecular weight of 92 kDa and a estimated reaction yield of 57%.

### Layer Building

Building of layers upon the cells, first of all the cells were incubated with Ac-TZ14011-Ad (1, 0° C., 15 min). After functionalizing the cell surface with a second laye,r using the host structure Cy3_{1.5}CD₁₇₂PIBMA₃₈₉, a third layer comprising of the guest structure Cy5_{0.5}Ad₁₅₀PIBMA₃₈₉ was introduced by incubation at 0° C. for 15 min. After each incubation step cells were washed with PBS. This is combined in the following incubation scheme:

TABLE 3
Scheme for the layer by layer labeling
off cells with consecutive polymers

| | t (min) | | | | | | | |
|---|---|---|---|---|---|---|---|---|
| | 0 | 15 | | 30 | 45 | | 60 | 75 |
| Pre-targeting | 1 | 2 | Wash | | | | | |
| layer | | | | | | | | |
| First layer of | 1 | 2 | Wash | 6 | Wash | | | |
| functionalization | | | | | | | | |
| Second layer of | 1 | 4 | Wash | 6 | 2 | Wash | | |
| functionalization | | | | | | | | |

To study the layer by layer functionalization, confocal images were taken of adhered cells comprising the three layers. The fluorescent signals of Cy3 and Cy5 showed co-localization with the previous layer and the fluorescent signal of the peptide functionalized CXCR4 receptors containing a GFP-tag.

### CLAUSES

1. A functionalized in vitro cell comprising a cell surface biomarker bound to a ligand, wherein said ligand is linked to a first guest molecule, and wherein said first guest molecule is non-covalently bound to a host functional group that is part of a first multivalent host structure, wherein the first multivalent host structure forms a first layer of functionalization.
2. A functionalized in vitro cell according to clause 1 comprising one or more further layers of functionalization, wherein subsequent layers of functionalization are formed by alternating layers of multivalent host and guest structures which are non-covalently bound to one another.
3. A functionalized in vitro cell according to clause 1 or 2, wherein an outer layer of functionalization formed by a multivalent host or guest structure, hereinafter referred to as outer layer of host-guest functionalization, is further functionalized with one or more functional end-groups.
4. A functionalized in vitro cell according to clause 3, wherein the interactions between host or guest functional groups in the outer layer of host-guest functionalization and the functional end-groups are non-covalent.
5. A functionalized in vitro cell according to clause 3 or 4, wherein a guest functional group which is present in the outer layer of host-guest functionalization is non-covalently bound to a host molecule that is linked to a functional end-group
6. A functionalized in vitro cell according clause 3 or 4, wherein a host functional group which is present in the outer layer of host-guest functionalization is non-covalently bound to a second guest molecule that is linked to a functional end-group.
7. A functionalized in vitro cell according to clause 1, wherein the first multivalent host structure is further functionalized with one or more functional end-groups.
8. A functionalized in vitro cell according to clause 1, 3 or 7, wherein a host functional group, which is present in said first multivalent host structure is non-covalently bound to a second guest molecule that is linked to a functional end-group.
9. A functionalized in vitro cell according to clause 1, wherein a host functional group of the first multivalent host structure is non-covalently bound to a guest functional group of a first multivalent guest structure, wherein the first multivalent guest structure forms a second layer of functionalization.
10. A functionalized in vitro cell according to clause 9, wherein the first multivalent guest structure is further functionalized with one or more functional end-groups.
11. A functionalized in vitro cell according to clause 9 or 10, wherein a guest functional group, which is present in said first multivalent guest structure is non-covalently bound to a host molecule that is linked to a functional end-group.
12. A functionalized in vitro cell according to clause 9, wherein a guest functional group of the first multivalent guest structure is non-covalently bound to a host functional group of a second multivalent host structure, wherein the second multivalent host structure forms a third layer of functionalization.
13. A functionalized in vitro cell according to clause 12, wherein the second multivalent host structure is further functionalized with one or more functional end-groups.
14. A functionalized in vitro cell according to clause 12 or 13, wherein a host functional group, which is present in said second multivalent host structure is non-covalently bound to a second guest molecule that is linked to a functional end-group.
15. A functionalized in vitro cell according to any of clauses 3-8, 10-11 or 13-14, wherein the one of more functional end-groups is selected from a group consisting of an imaging label, a targeting group, a therapeutic group, a nanoparticle, an organic surface, an inorganic surface, a surface of another cell, or a cloaking group.
16. A functionalized in vitro cell according to any of clauses 3-8, 10-11 or 13-15, wherein the one or more functional end-groups comprises one or more types of functional end-groups.
17. A functionalized in vitro cell according to any of the preceding clauses, wherein said cell comprises a plurality of cell surface biomarkers each bound to a respective ligand, wherein said ligand is linked to a first guest molecule, and wherein said first guest molecule is non-covalently bound to a host functional group of a multivalent host structure, said multivalent host structure forming a first layer of functionalization.
18. A functionalized in vitro cell according to clause 17, wherein the plurality of cell surface biomarkers comprises one or more types of cell surface biomarkers.
19. A functionalized in vitro cell according to clause 17 or 18, wherein the plurality of cell surface biomarkers comprise essentially all cell surface biomarkers of a given type of cell surface biomarker.
20. A functionalized in vitro cell according to any of the preceding clauses, wherein the first multivalent host structure is connected to at least two different cell surface biomarkers via its non-covalent binding to at least two guest molecules, wherein the at least two linked guest molecules are linked to a respective ligand which is bound to a respective receptor.
21. A functionalized in vitro cell according to any of the preceding clauses, wherein the first multivalent host structure comprises at least three, such as, e.g., at least four, at least five, at least six, at least seven, at least eight, at least nine or at least 10 host functional groups.
22. A functionalized in vitro cell according to any of the preceding clauses, wherein the first multivalent host structure comprises a scaffold polymer.
23. A functionalized in vitro cell according to clause 22, wherein the scaffold polymer comprises at least about 5, such as at least about 10, at least about 15, at least about 20, at least about 25, at least about 30, at least about 35 or at least about 40 monomer repeat units.
24. A functionalized in vitro cell according to clause 22 or 23, wherein the scaffold polymer is poly(isobutylene-alt-maleic anhydride) (PIBMA).
25. A functionalized in vitro cell according to any of the preceding clauses, wherein the first multivalent guest structure comprises a scaffold molecule.
26. A functionalized in vitro cell according to clause 25, wherein the scaffold molecule is a polypeptide comprising less than about 30, such as, e.g. less than about 25, less than about 20, less than about 15, less than about 10, less than about 5 or less than about 6 amino acids.
27. A functionalized in vitro cell according to clause 25, wherein the scaffold molecule is an oligopeptide, such as e.g. a dipeptide, a tripeptide, a tetrapeptide or a pentapeptide.
28. A functionalized in vitro cell according to clause 26 or 27, wherein the repeat unit of the polypeptide or oligopeptide is β-alanine.
29. A functionalized in vitro cell according to clause 1, wherein a host functional group, which is present in said first multivalent host structure is non-covalently bound to a guest functional group that is part of a first multivalent guest structure.
30. An in vitro cell according to claim 29, wherein a guest functional group, which is present in said first multivalent guest structure and which is not bound to a host functional group in said first multivalent host structure, is non-covalently bound to a host molecule that is linked to a functional end-group.
31. An in vitro cell according to claim 1, wherein a host functional group, which is present in said first multivalent host structure and which is not bound to a first guest molecule, is non-covalently bound to a second guest molecule that is linked to a functional end-group.
32. An in vitro cell according to clauses 30 or 31, wherein said functional end-group is an imaging label, a targeting group, a therapeutic group, a nanoparticle, an organic surface, an inorganic surface, a surface of another cell, or a cloaking group.
33. An in vitro cell according to any one the preceding clauses, wherein said cell surface biomarker is a receptor.
34. An in vitro cell according clause 33, wherein said receptor is the CXCR4 receptor and wherein said ligand is Ac-TZ14011.
35. An in vitro cell according to any one of any of the preceding clauses, wherein said first guest molecule is adamantane and said host functional group of the first multivalent host structure is the host functional group of a cyclodextrin.
36. An in vitro cell according to any one the preceding clauses, wherein said guest functional group present the first multivalent guest structure is the guest functional group of adamantane and said host functional group in the first multivalent host structure is the host functional group of a cyclodextrin.
37. A functionalized in vitro cell as defined in any of clauses 1-36 for use in therapy.
38. A functionalized in vitro cell as defined in any of clauses 1-36 for use in cell therapy, stem cell therapy, immunotherapy or tissue building.
39. Use of functionalized in vitro cells as defined in any of clauses 1-36 for imaging, sensing or purification of said cell.
40. Use of functionalized in vitro cells as defined in any of clauses 1-36 for inducing cell-cell interactions, for building tissue, drug delivery.
41. Method for treating a disease, the method comprising targeting a cell as defined in any of clauses 1-36 to a cell or a tissue in which the presence of the targeted as a curing or preventing effect on said disease.
42. A method for functionalizing cells, comprising the steps of:
a) Maintaining a selection of cells in vitro, wherein said cells are selected based on the presence of a cell surface biomarker of choice on the cell surface of said cells;
b) Incubating said cells with a ligand that is able to bind to said cell surface biomarker, wherein said ligand is linked to a first guest molecule;
c) Allowing the ligand to interact with said cell surface biomarker;
d) Incubating said cells with a first multivalent host structure; and
e) Allowing the first multivalent host structure to non-covalently interact with said first guest molecule; in order to obtain functionalized in vitro cells comprising a first layer of functionalization.
43. A method according to clause 42, further comprising the steps of:
f) Incubating the cells resulting of step e) with a first multivalent guest structure; and
g) Allowing said first multivalent guest structure to non-covalently interact with the first multivalent host structure;
in order to obtain functionalized in vitro cells comprising a second layer of functionalization.
44. A method according to clause 43, further comprising the steps of:
h) Incubating the cells resulting of step g) with a second multivalent host structure; and
i) Allowing the second multivalent host structure to non-covalently interact with said first multivalent guest structure;
in order to obtain functionalized in vitro cells comprising a third layer of functionalization.
45. A method according to any of clauses 42 or 44, further comprising the steps of:
j) Incubating the cells resulting from any of steps e) or i) with a second guest molecule that is linked to a functional end-group; and
g) Allowing said second guest molecule to non-covalently interact with said first or second multivalent host structure.
46. A method according to clause 43, further comprising the steps of:
h) Incubating the cells resulting of step g) with a host molecule that is linked to a functional end-group; and
i) Allowing said host molecule to non-covalently interact with said first multivalent guest structure.
47. A method according to clause 45 or 46, wherein said functional end-group is an imaging label, a targeting group, a therapeutic group, a nanoparticle, an organic surface, an inorganic surface, a surface of another cell, or a cloaking group.
48. A functionalized in vitro cell obtainable by the method defined in any of clauses 44-47.
49. Method for sensing cells in a solution the method comprising the steps of:
a) Functionalizing the cells subject to sensing by the method defined in any of clauses 44-48; and
b) Subjecting the cells to sensing of the introduced functional groups on the surface of said cells.
50. Method for purification of cells from a solution the method comprising the steps of:
a) Functionalizing the cells subject to purification by the method defined in any of clauses 44-48; and
b) Subjecting the cells to purification by interaction with the introduced functional groups on the surface of said cell.
51. Method for diagnosing a disease the method comprising the steps of:
a) Functionalizing the cells relevant for the diagnosis of a disease in an in vitro sample by the method defined in any of clauses 44-48; and
b) Subjecting the cells to detection of the introduced functional groups on the surface of said cell.
52. Method for imaging of cells the method comprising the steps of:
a) Functionalizing the cells relevant for imaging by the method defined in any clauses 44-48, wherein at least one of the one or more functional end-groups is an imaging label; and
b) Subjecting the cells to imaging of the introduced functional groups on the surface of said cell.
53. Method for inducing cell-cell interactions the method comprising the steps of:
a) Functionalizing two groups of cells by the method defined in any of clauses 44-48, such that the outer layer of functionalization of the first group is complementary to the outer layer of functionalization of the second group; and
b) Allowing the two groups of cells to interact with one another.
54. Method for targeting cells to an organic surface, an inorganic surface, or a surface of another cell, the method comprising the steps of:
a) Functionalizing the cells relevant for targeting by the method defined in any of clauses 44-48; and
b) Targeting said cells to the organic surface, inorganic surface, or surface of another cell, wherein said surfaces comprise a target recognized by the one or more targeting groups introduced on the surface of the cell.
55. A method of targeting selected cells to an organic surface, an inorganic surface or a surface of another cell, said method comprising the steps of:
a) Selecting cells based on the presence of a cell surface biomarker of choice on the cell surface of said selected cells;
b) Optionally maintaining or culturing said selected cells in vitro;
c) Allowing the interaction between said selected cells and a ligand that is able to bind to said cell surface biomarker, wherein said ligand is specific for said cell surface biomarker and is linked to a first guest molecule;
d) Allowing the non-covalent interaction between a first multivalent host structure and said first guest molecule; and either of the following steps:
1) Targeting the cells obtained from step d) to said organic surface, said inorganic surface or said surface of another cell, wherein the surface of said organic surface, said inorganic surface or said surface of another cell comprises guest functional groups; or
2) a) Allowing the interaction between a second guest molecule with said first multivalent host structure, wherein said second guest molecule is linked to a targeting group that can specifically bind to an organic surface, an inorganic surface or a surface of another cell; and b) Targeting the cells obtained from step 2) a) to said organic surface, said inorganic surface or said surface of another cell, wherein the surface of said organic surface, said inorganic surface or said surface of another cell comprises a target of the targeting group.
56. A compound with the chemical formula of

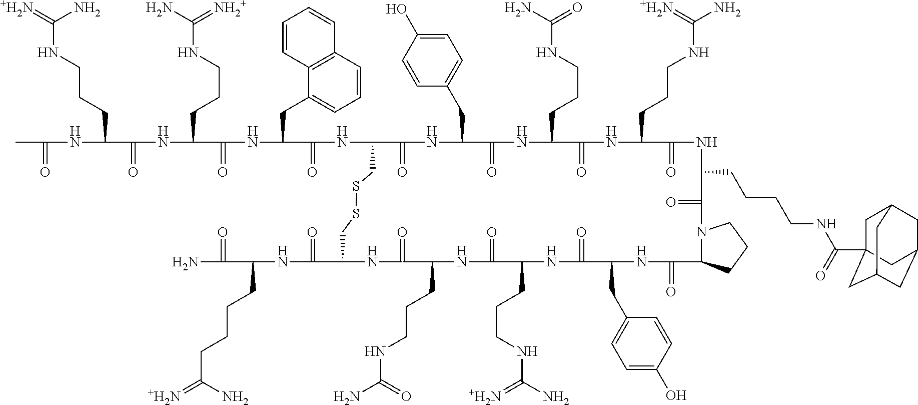 [SMILES: CC(=O)N[C@H](CCCNC(N)=[NH2+])C(=O)N[C@H](CCCNC(N)=[NH2+])C(=O)N[C@H](Cc1cccc2ccccc12)C(=O)N[C@@H]1CSSC[C@H](C(=O)N[C@H](CCCCC(N)=[NH2+])C(N)=O)NC(=O)[C@@H](CCCNC(N)=O)NC(=O)[C@@H](CCCNC(N)=[NH2+])NC(=O)[C@@H](Cc2ccc(O)cc2)NC(=O)[C@@H]2CCCN2C(=O)[C@H](CCCCNC(=O)C23CC4CC(CC(C4)C2)C3)NC(=O)[C@@H](CCCNC(N)=[NH2+])NC(=O)[C@@H](CCCNC(N)=O)NC(=O)[C@@H](Cc2ccc(O)cc2)NC1=O]

1. A functionalized in vitro cell comprising a cell surface biomarker bound to a ligand, wherein said ligand is linked to a first guest molecule, and wherein said first guest molecule is non-covalently bound to a host functional group that is part of a first multivalent host structure, wherein the first multivalent host structure forms a first layer of functionalization.

2. The functionalized in vitro cell according to claim 1 comprising one or more further layers of functionalization, wherein subsequent layers of functionalization are formed by alternating layers of multivalent host and guest structures which are non-covalently bound to one another.

3. The functionalized in vitro cell according to claim 1, wherein an outer layer of functionalization formed by a multivalent host or guest structure, hereinafter referred to as outer layer of host-guest functionalization, is further functionalized with one or more functional end-groups.

4. The functionalized in vitro cell according to claim 1, wherein a host functional group, which is present in said first multivalent host structure is non-covalently bound to a second guest molecule that is linked to a functional end-group.

5. The functionalized in vitro cell according to claim 1, wherein a host functional group of the first multivalent host structure is non-covalently bound to a guest functional group of a first multivalent guest structure, wherein the first multivalent guest structure forms a second layer of functionalization.

6. The functionalized in vitro cell according to claim 5, wherein a guest functional group, which is present in said first multivalent guest structure is non-covalently bound to a host molecule that is linked to a functional end-group.

7. The functionalized in vitro cell according to claim 3, wherein one or more functional end-groups are selected from a group consisting of an imaging label, a targeting group, a therapeutic group, a nanoparticle, an organic surface, an inorganic surface, a surface of another cell, or a cloaking group, or comprises one or more types of functional end-groups.

8. (canceled)

9. The functionalized in vitro cell according to claim 1, wherein said cell comprises a plurality of cell surface biomarkers each bound to a respective ligand, wherein said ligand is linked to a first guest molecule, and wherein said first guest molecule is non-covalently bound to a host functional group of a multivalent host structure, said multivalent host structure forming a first layer of functionalization.

10. The functionalized in vitro cell according to claim 9, wherein the plurality of cell surface biomarkers comprises one or more types of cell surface biomarkers.

11. The functionalized in vitro cell according to any of the preceding claims, wherein the first multivalent host structure comprises a scaffold polymer or a scaffold molecule.

12. (canceled)

13. The functionalized in vitro cell according to claim 1 for use in therapy.

14. A method for functionalizing cells, comprising the steps of:
a) Maintaining a selection of cells in vitro, wherein said cells are selected based on the presence of a cell surface biomarker of choice on the cell surface of said cells;
b) Incubating said cells with a ligand that is able to bind to said cell surface biomarker, wherein said ligand is linked to a first guest molecule;
c) Allowing the ligand to interact with said cell surface biomarker;
d) Incubating said cells with a first multivalent host structure; and
e) Allowing the first multivalent host structure to non-covalently interact with said first guest molecule; in order to obtain functionalized in vitro cells comprising a first layer of functionalization, and optionally,
j) Incubating the cells resulting from step e) with a second guest molecule that is linked to a functional end-group; and
k) Allowing said second guest molecule to non-covalently interact with said first or second multivalent host structure.

15. The method according to claim 14, further comprising the steps of:
f) Incubating the cells resulting of step e) with a first multivalent guest structure; and
g) Allowing said first multivalent guest structure to non-covalently interact with the first multivalent host structure;
in order to obtain functionalized in vitro cells comprising a second layer of functionalization, and optionally, further comprising the steps of:
h) Incubating the cells resulting of step g) with a host molecule that is linked to a functional end-group; and
i) Allowing said host molecule to non-covalently interact with said first multivalent guest structure.

16. The method according to claim 15, further comprising the steps of:
h) Incubating the cells resulting of step g) with a second multivalent host structure; and
i) Allowing the second multivalent host structure to non-covalently interact with said first multivalent guest structure;
in order to obtain functionalized in vitro cells comprising a third layer of functionalization, and optionally,
j) Incubating the cells resulting from step i) with a second guest molecule that is linked to a functional end-group; and
k) Allowing said second guest molecule to non-covalently interact with said first or second multivalent host structure.

17. (canceled)

18. (canceled)

19. The method according to claim 17, wherein said functional end-group is an imaging label, a targeting group, a therapeutic group, a nanoparticle, an organic surface, an inorganic surface, a surface of another cell, or a cloaking group.

20. A functionalized in vitro cell produced by the method defined in claim 14.

21. A method for diagnosing a disease the method comprising the steps of:
a) Functionalizing the cells relevant for the diagnosis of a disease in an in vitro sample by the method defined in claim 14; and
b) Subjecting the cells to detection of the introduced functional groups on the surface of said cell.

22. A method for imaging of cells the method comprising the steps of:
a) Functionalizing the cells relevant for imaging by the method defined in claim 14, wherein at least one of the one or more functional end-groups is an imaging label; and
b) Subjecting the cells to imaging of the introduced functional groups on the surface of said cell.

23. A method for inducing cell-cell interactions the method comprising the steps of:
a) Functionalizing two groups of cells by the method defined in claim 14, such that the outer layer of functionalization of the first group is complementary to the outer layer of functionalization of the second group: and
b) Allowing the two groups of cells to interact with one another.

24. A method for targeting cells to an organic surface, an inorganic surface, or a surface of another cell, the method comprising the steps of:
a) Functionalizing the cells relevant for targeting by the method defined in claim 14; and
b) Targeting said cells to the organic surface, inorganic surface, or surface of another cell, wherein said surfaces comprise a target recognized by the one or more targeting groups introduced on the surface of the cell.
